(19) United States
(12) Patent Application Publication
(10) Pub. No.: US 2004/0152747 A1
(43) Pub. Date: Aug. 5, 2004

(54) Thiazole compounds for treatment of neurodegenerative disorders

(75) Inventors: Yuhpyng L. Chen, Waterford, CT (US); Michael L. Corman, Ledyard, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/682,686

(22) Filed: Oct. 8, 2003

Related U.S. Application Data
(60) Provisional application No. 60/417,400, filed on Oct. 9, 2002.

(51) Int. Cl. A61K 31/427; A61K 31/426; C07D 417/02

(52) U.S. Cl. 514/370; 548/181; 548/190; 514/342

(57) ABSTRACT

The invention provides compounds of Formula I:

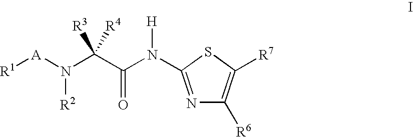

wherein R¹, R², R³, R⁴, R⁶, R⁷, and A are as defined. Compounds of Formula I have activity inhibiting production of Aβ-peptide. The invention also provides pharmaceutical compositions and methods for treating diseases, for example Alzheimer's disease, in mammals comprising compounds of Formula I.

### FIELD OF THE INVENTION

The present invention relates to treatment of Alzheimer's disease and other neurodegenerative disorders in mammals, including in humans. This invention also relates to inhibiting in mammals, including in humans, the production of Aβ-peptides which can contribute to formation of neurological deposits of amyloid protein. More particularly, this invention relates to thiazole compounds useful for treatment of neurological disorders, such as Alzheimer's disease and Down's Syndrome, related to Aβ-peptide production.

### BACKGROUND OF THE INVENTION

Dementia results from a wide variety of distinctive pathological processes. The most common pathological processes causing dementia are Alzheimer's disease (AD), cerebral amyloid angiopathy (CAA) and prion-mediated diseases (see, e.g., Haan et al. Clin. Neurol. Neurosurg. 1990, 92(4):305-310; Glenner et al. J Neurol. Sci. 1989, 94:1-28). AD affects nearly half of all people past the age of 85, the most rapidly growing portion of the United States population. As such, the number of AD patients in the United States is expected to increase from about 4 million to about 14 million by the middle of the next century.

Treatment of AD typically is the support provided by a family member in attendance. Stimulated memory exercises on a regular basis have been shown to slow, but not stop, memory loss. A few drugs, for example Aricept™, provide treatment of AD.

A hallmark of AD is the accumulation in the brain of extracellular insoluble deposits called amyloid plaques and abnormal lesions within neuronal cells called neurofibrillary tangles. Increased plaque formation is associated with an increased risk of AD. Indeed, the presence of amyloid plaques, together with neurofibrillary tangles, are the basis for definitive pathological diagnosis of AD.

The major components of amyloid plaques are the amyloid Aβ-peptides, also called Aβ-peptides, which consist of three proteins having 40, 42 or 43 amino acids, designated as the Aβ₁₋₄₀, Aβ₁₋₄₂, and Aβ₁₋₄₃ peptides, respectively. The Aβ-peptides are thought to cause nerve cell destruction, in part, because they are toxic to neurons in vitro and in vivo.

The Aβ peptides are derived from larger amyloid precursor proteins (APP proteins), which consist of four proteins containing 695, 714, 751 or 771 amino acids, designated as the APP₆₉₅, APP₇₁₄, APP₇₅₁ and APP₇₇₁, respectively. Proteases are believed to produce the Aβ peptides by cleaving specific amino acid sequences within the various APP proteins. The proteases are named “secretases” because the Aβ-peptides they produce are secreted by cells into the extracellular environment. These secretases are each named according to the cleavage(s) they make to produce the Aβ-peptides. The secretase that forms the amino terminal end of the Aβ-peptides is called the beta-secretase. The secretase that forms the carboxyl terminal end of the Aβ-peptides is called the gamma-secretase (Haass, C. and Selkoe, D. J. 1993 Cell 75:1039-1042).

This invention relates to novel compounds that inhibit Aβ-peptide production, to pharmaceutical compositions comprising such compounds, and to methods of using such compounds to treat neurodegenerative disorders.

### SUMMARY OF THE INVENTION

The present invention provides compounds of Formula:

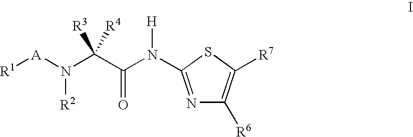

wherein:

A is selected from —C(═O)C(═O)—, —C(═O)NR⁹—, —C(═O)Z-, —C(═S)Z-, —C(═NR⁵)Z-, and —S(O)₂—;

wherein Z is —CH₂—, —CH(OH)—, —CH(OC(═O)R¹¹)—, —CH(NR⁹R¹⁰)—, —CH(CH₂(OH))—, —CH(CH(C₁-C₄ alkyl)(OH))—, or —CH(C(C₁-C₄ alkyl)(C₁-C₄ alkyl)(OH))—, for example —CH(C(CH₃)(CH₃)(OH))— or —CH(C(CH₃)(CH₂CH₃)(OH))—;

R¹ is selected from C₁-C₂₀ alkyl and —C₁-C₂₀ alkoxy, C₃-C₈ cycloalkyl, (C₄-C₈)cycloalkenyl, (C₅-C₁₁)bi- or tricycloalkyl, (C₇-C₁₁)bi- or tricycloalkenyl, (3-8 membered) heterocycloalkyl, (C₆-C₁₄)aryl, or (5-14 membered) heteroaryl, wherein said alkyl and alkoxy each optionally contains from one to five double or triple bonds, and wherein each hydrogen atom of said alkyl and alkoxy is optionally replaced with a fluorine;

wherein when R¹ is alkyl or alkoxy, R¹ is optionally substituted with from one to three substituents R^{1a}, and wherein when R¹ is cycloalkyl, cycloalkenyl, bi- or tricycloalkyl, bi- or tricycloalkenyl, heterocycloalkyl, aryl, or heteroaryl, then R¹ is optionally substituted with from one to three substituents R^{1b};

R^{1a} is in each instance independently selected from —OH, —C₁-C₆ alkyl independently optionally containing from one to three double or triple bonds, —C₁-C₆ alkoxy independently optionally containing from one to three double or triple bonds, —Cl, —F, —Br, —I, —CN, —NO₂, —NR⁹R¹⁰, —C(═O)NR⁹R¹⁰, —S(O)ₙNR⁹R¹⁰, —C(═O)R¹¹, —S(O)ₙR¹¹, —C(═O)OR¹², —C₃-C₈ cycloalkyl, —C₄-C₈ cycloalkenyl, -(C₅-C₁₁)bi- or tricycloalkyl, -(C₇-C₁₁)bi- or tricycloalkenyl, -(3-8 membered) heterocycloalkyl, -(C₆-C₁₄)aryl, -(5-14 membered) heteroaryl, -(C₆-C₁₄) aryloxy, and -(5-14 membered) heteroaryloxy, wherein said alkyl, alkoxy, cycloalkyl, cycloalkenyl, bi- or tricycloalkyl, bi- or tricycloalkenyl, heterocycloalkyl, aryl, heteroaryl, aryloxy, and heteroaryloxy are each independently optionally substituted with from one to three substituents R^{1b};

R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, -(5-15 membered) heteroaryl, and —C₁-C₆ alkyl independently optionally containing from one to three double or triple bonds and independently substituted with from one to six atoms independently selected from F, Cl, Br, and I;

R² is selected from —H, —C₁-C₄ alkyl optionally containing one or two double or triple bonds, —C(═O)(C₁-C₄ alkyl), —C₆-C₁₀ aryl, —SO₂—(C₆-C₁₀ aryl), and —SO₂—CH₂—(C₆-C₁₀ aryl), and R² is optionally substituted with from one to three substituents R^{1b};

R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);

R⁴ is H, D, F, or C₁-C₄ alkyl;

or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;

R⁵ is selected from —H, —C₁-C₆ alkyl optionally substituted with from one to three R^{1a}, and —C₆-C₁₀ aryl optionally substituted with from one to three R^{1a};

or R⁵ and R¹ may together optionally form a five to fourteen membered heteroaryl ring or a five to eight membered heterocycloalkyl ring, wherein said heteroaryl ring optionally contains one or two further heteroatoms independently selected from N, O, and S and said heterocycloalkyl ring optionally contains one or two further heteroatoms independently selected from N—R⁹, O, and S(O)_{zero-2}, and wherein said heterocycloalkyl ring optionally contains from one to three double bonds, and wherein said heteroaryl or heterocycloalkyl ring is optionally substituted from one to three substituents R^{1b};

R⁶ is selected from —H, —C₁-C₂₀ alkyl, —Cl, —F, —Br, —I, —CN, —CF₃, —C(═O)R¹¹, —C(═O)OR¹², —S(O)ₙNR⁹R¹⁰, —S(O)ₙR¹¹, —C(═NR⁹)R¹⁵, -(C₃-C₁₂) cycloalkyl, -(C₄-C₁₂) cycloalkenyl, and —C₆-C₁₀ aryl, wherein said alkyl, alkylene, cycloalkyl, cycloalkenyl, and aryl of R⁶ are each optionally substituted with from one to three substituents R^{1b};

R⁷ is selected from H, —Cl, —F, —Br, —I, —CN, —NO₂, —NR¹⁴R¹⁵, —CF₃, —C(═O)NR¹⁴R¹⁵, —C(═O)R¹³, —S(O)ₙR¹³, —C(═O)OR¹³, —C(═NR⁹)R¹⁵, —S(O)ₙNR¹⁴R¹⁵, —C₁-C₂₀ alkyl, —C₁-C₂₀ alkoxy, -(C_{zero}-C₄ alkylene)-(C₃-C₁₂ cycloalkyl), -(C_{zero}-C₄ alkylene)-((C₄-C₁₂)cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-((3-12 membered) heterocycloalkyl), -(C_{zero}-C₄ alkylene)-((7-20 membered) heterobi- or heterotricycloalkyl), -(C_{zero}-C₄ alkylene)-((C₆-C₁₄)aryl), and -(C_{zero}-C₄ alkylene)-((5-15 membered) heteroaryl); wherein R⁷ is optionally substituted with from one to three substituents independently selected from R^{1a}, -(CH₂)₁₋₁₀NR⁹R¹⁰, —C₃-C₁₂ cycloalkyl, -((4-12 membered) heterocycloalkyl), -(C₆-C₁₄) aryl, -((5-15 membered) heteroaryl), -(4-12 membered) heterocycloalkoxy), -(C₆-C₁₂) aryloxy and -((5-12 membered) heteroaryloxy); said cycloalkyl, cycloalkenyl, bi- or tricycloalkyl, bi- or tricycloalkenyl, heterocycloalkyl, aryl, and heteroaryl of R⁷ are each optionally and independently substituted with from one to six F; said alkyl, alkoxy, and alkylene of R⁷ each optionally contains from one to five double or triple bonds; and each hydrogen atom of said alkyl, alkoxy, and alkylene of R⁷ is independently optionally replaced with a fluorine;

or R⁶ and R⁷ may together optionally form a -(C₆-C₁₀) aryl ring, -(C₆-C₈) cycloalkyl or cycloalkenyl ring, a five to eight membered heterocycloalkyl or heterocycloalkenyl ring, a -(C₁₀-C₁₄) membered bicycloalkyl or bicycloalkenyl ring, or a ten to fourteen membered heterobicycloalkyl or heterobicycloalkenyl ring fused to the thiazole ring of Formula I, wherein from one to three members of said heterocycloalkyl and heterocycloalkenyl rings, and from one to five members of said heterobicycloalkyl and heterobicycloalkenyl rings are selected independently from N—R⁹, O and S(O)_{zero-2}, and wherein said aryl, cycloalkyl, cycloalkenyl, heterocycloalkyl, heterocycloalkenyl, bicycloalkyl, bicycloalkenyl, heterobicycloalkyl, and heterobicycloalkenyl rings optionally are substituted with from one to three R^{1b};

R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇-C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;

or NR⁹R¹⁰ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;

R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};

R¹³ is selected from H, —C₁-C₆ alkyl optionally containing from one to three double or triple bonds and wherein each hydrogen is independently optionally replaced with a fluorine, -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), and -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-((3-12 membered) heterocycloalkyl), -(C_{zero}-C₄ alkylene)-((7-20 membered) heterobi- or heterotricycloalkyl), and -(C_{zero}-C₄ alkylene)-((5-14 membered) heteroaryl), and R¹³ is optionally substituted with from one to three substituents R^{1b};

R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;

or NR¹⁴R¹⁵ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I; and

n is in each instance an integer independently selected from zero, 1, 2, and 3;

and pharmaceutically-acceptable salts thereof.

Compounds of Formula I inhibit production of Aβ-peptide. Compounds of Formula I and their pharmaceutically acceptable salts are therefore useful in treating neurodegenerative disorders, for example AD, in mammals, including humans.

In one embodiment, the present invention provides compounds of Formula I wherein A is —C(═O)Z- or —C(═O)C(═O)—. If A is —C(═O)Z-, then Z is preferably —CH₂— or —CH(OH)—.

In another embodiment, Z is —CH(NH₂)—.

In another embodiment, the invention provides compounds of Formula I wherein R³ is C₁-C₄ alkyl wherein each hydrogen is independently optionally replaced with a fluorine. In another embodiment R³ is allyl. In another embodiment R³ is methyl, ethyl, n-propyl, n-butyl, i-butyl, s-butyl, or —CH₂CH₂SCH₃.

In another embodiment, the present invention provides compounds of Formula I wherein R⁶ is selected from hydrogen, methyl, ethyl, —F, —Cl, —Br, and —CF₃.

In another embodiment the present invention provides compounds of Formula I wherein R¹ is —C₂-C₁₂ alkyl, C₃-C₈ cycloalkyl, (C₅-C₈)cycloalkenyl, -(C₅-C₁₁)bi- or tricycloalkyl, -(C₇-C₁₁)bi- or tricycloalkenyl, (3-8 membered) heterocycloalkyl), -(C₆-C₁₀)aryl, -(5-10 membered) heteroaryl, or C₁-C₄ alkyl substituted with R^{1a} wherein R^{1a} is -(C₆-C₁₀)aryl or -(5-10 membered) heteroaryl.

In another embodiment, the present invention provides compounds of Formula I wherein R¹ is C₂-C₁₀ alkyl, C₃-C₁₀ cycloalkyl, or -(C₇-C₁₁)bicycloalkyl, wherein said alkyl optionally contains from one to five double bonds, and wherein each hydrogen atom of said alkyl may optionally be replaced with a fluorine.

When R¹ is C₂-C₁₀ alkyl, in one embodiment, R¹ is straight-chain. In another embodiment when R¹ is C₂-C₁₀ alkyl, R¹ is branched C₃-C₁₀ alkyl.

In another embodiment, R¹ is C₃-C₁₀ alkyl comprising a tertiary carbon, for example i-propyl or 2-methylpropyl. In another embodiment, R¹ is C₄-C₁₀ alkyl comprising a quaternary carbon, for example t-butyl.

In a further embodiment, R¹ is selected from phenyl, thienyl, and pyridyl, optionally and independently substituted with one or two substituents R^{1b}. When R¹ is phenyl, thienyl, or pyridyl substituted optionally with one or two substituents R^{1b}, then each R^{1b} is preferably independently selected from —C₁-C₄ alkyl (in different embodiments, independently optionally containing one or two double or triple bonds), CF₃, —C₁-C₄ alkoxy (in different embodiments, independently optionally containing one or two double or triple bonds), —F, —Cl, —Br, phenyl, and phenoxy.

In a further embodiment, R¹ is phenyl or pyridyl and is optionally substituted with one or two substituents R^{1b} independently selected from —F, —Cl and —CF₃.

In another embodiment R¹ is C₃-C₇ cycloalkyl, for example [2.2.1]-heptanyl.

In each of the aforementioned embodiments, A is preferably —C(═O)Z- or —C(═O)C(═O)—, Z preferably being —CH₂— or —CH(OH)—. Furthermore, R³ is preferably C₁-C₄ alkyl, for example methyl, ethyl, n-propyl, n-butyl, i-butyl, s-butyl, or R³ is allyl or —CH₂CH₂SCH₃, and R⁶ is preferably hydrogen, methyl, ethyl, —F, —Cl, —Br, and —CF₃.

In a further embodiment, A is —C(═O)Z- or —C(═O)C(═O)—; Z is —CH₂— or —CH(OH)—; R³ is C₁-C₄ alkyl wherein each hydrogen is independently optionally replaced with a fluorine, or R³ is allyl or —CH₂CH₂SCH₃; R⁶ is selected from hydrogen, methyl, ethyl, —F, —Cl, —Br, and —CF₃; and R¹ is —C₂-C₁₂ alkyl, C₃-C₈ cycloalkyl, (C₅-C₈)cycloalkenyl, -(C₅-C₁₁)bi- or tricycloalkyl, -(C₇-C₁₁l)bi- or tricycloalkenyl, -((3-8 membered) heterocycloalkyl), -(C₆-C₁₀)aryl, -(5-10 membered) heteroaryl, or C₁-C₄ alkyl substituted with R^{1a} wherein R^{1a} is -(C₆-C₁₀)aryl or -(5-10 membered) heteroaryl.

In another embodiment, the present invention provides compounds of Formula I wherein A is —C(═O)Z- or —C(═O)C(═O)—; Z is —CH₂— or —CH(OH)—; R³ is C₁-C₄ alkyl wherein each hydrogen is independently optionally replaced with a fluorine, or R³ is allyl or —CH₂CH₂SCH₃; R⁶ is selected from hydrogen, methyl, ethyl, —F, —Cl, —Br, and —CF₃; and R¹ is C₂-C₁₀ alkyl, C₃-C₁₀ cycloalkyl, or -(C₇-C₁₁)bicycloalkyl, wherein said alkyl optionally contains from one to five double bonds, and wherein each hydrogen atom of said alkyl is optionally replaced with a fluorine.

In another embodiment, the invention provides compounds of Formula I wherein A is —C(═O)Z- or —C(═O)C(═O)—; Z is —CH₂— or —CH(OH)—; R³ is C₁-C₄ alkyl wherein each hydrogen is independently optionally replaced with a fluorine, or R³ is allyl or —CH₂CH₂SCH₃; R⁶ is selected from hydrogen, methyl, ethyl, —F, —Cl, —Br, and —CF₃; and R¹ is straight chain C₂-C₁₀ alkyl or branched C₃-C₁₀ alkyl.

In another embodiment, A is —C(═O)Z- or —C(═O)C(═O)—; Z is —CH₂— or —CH(OH)—; R³ is C₁-C₄ alkyl wherein each hydrogen is independently optionally replaced with a fluorine, or R³ is allyl or —CH₂CH₂SCH₃; R⁶ is selected from hydrogen, methyl, ethyl, —F, —Cl, —Br, and —CF₃; and R¹ is C₃-C₁₀ alkyl comprising a tertiary carbon, for example i-propyl or 2-methylpropyl, or R¹ is C₄-C₁₀ alkyl comprising a quaternary carbon, for example t-butyl.

In a further embodiment, A is —C(═O)Z- or —C(═O)C(═O)—; Z is —CH₂— or —CH(OH)—; R³ is C₁-C₄ alkyl wherein each hydrogen is independently optionally replaced with a fluorine, or R³ is allyl or —CH₂CH₂SCH₃; R⁶ is selected from hydrogen, methyl, ethyl, —F, —Cl, —Br, and —CF₃; and R¹ is selected from phenyl, thienyl, and pyridyl, optionally and independently substituted with one or two substituents R^{1b}, preferably independently selected from —C₁-C₄ alkyl, CF₃, —C₁-C₄ alkyoxy, —F, —Cl, —Br, phenyl, and phenoxy.

In a further embodiment, A is —C(═O)Z- or —C(═O)C(═O)—; Z is —CH₂— or —CH(OH)—; R³ is C₁-C₄ alkyl wherein each hydrogen is independently optionally replaced with a fluorine, or R³ is allyl or —CH₂CH₂SCH₃; R⁶ is selected from hydrogen, methyl, ethyl, —F, —Cl, —Br, and —CF₃; and R¹ is phenyl or pyridyl and is optionally substituted with one or two substituents R^{1b} independently selected from —F, —Cl and —CF₃.

In another embodiment, A is —C(═O)Z- or —C(═O)C(═O)—; Z is —CH₂— or —CH(OH)—; R³ is C₁-C₄ alkyl wherein each hydrogen is independently optionally replaced with a fluorine, or R³ is allyl or —CH₂CH₂SCH₃; R⁶ is selected from hydrogen, methyl, ethyl, —F, —Cl, —Br, and —CF₃; and R¹ is C₃-C₇ cycloalkyl, for example [2.2.1]-heptanyl.

In another embodiment, this invention provides compounds of Formula I wherein R⁷ is selected from —H, —C₁-C₁₂ alkyl optionally containing from one to five double bonds and wherein each hydrogen is independently optionally replaced with a fluorine, —C₁-C₂₀ alkoxy optionally containing from one to five double bonds and wherein each hydrogen is independently optionally replaced with a fluorine, —F, —Cl, —Br, —I, —CN, —NO₂, -(C₃-C₁₂) cycloalkyl optionally substituted with from one to six fluorine, -((3-12 membered) heterocycloalkyl) optionally substituted with from one to six fluorine, -(C₆-C₁₄) aryl, -((5-15 membered) heteroaryl), —CHO, —C(═O)(C₁-C₁₅ alkyl), —C(═O)((5-12 membered)heterocycloalkyl), —C(═O)(C₆-C₁₄ aryl), —C(═O)((5-15 membered) heteroaryl), —C(═O)(C₅-C₁₂ cycloalkyl), —C(═O)O(C₁-C₈ alkyl), —C(═O)N(C₁-C₁₀ alkyl)(C₁-C₁₀ alkyl), —C(═O)N(C₁-C₁₀ alkyl)(C₆-C₁₀ aryl), —C(═O)NH(C₆-C₁₀ aryl), —C(═O)N(C₁-C₁₀ alkyl)((5-10 membered) heteroaryl), —C(═O)NH((5-10 membered) heteroaryl), —C(═O)N(C₁-C₁₀ alkyl)((5-10 membered) heterocycloalkyl), —C(═O)NH((5-10 membered) heterocycloalkyl), —C(═O)N(C₁-C₁₀ alkyl)(C₅-C₁₀ cycloalkyl), —C(═O)NH(C₅-C₁₀ cycloalkyl), —S(O)ₙ(C₁-C₁₅ alkyl), —S(O)ₙ(C₅-C₁₂ cycloalkyl), —S(O)ₙ(C₆-C₁₅ aryl), —S(O)ₙ((5-10 membered) heteroaryl), wherein said alkyl, cycloalkyl, heterocycloalkyl, aryl and heteroaryl are each optionally substituted with from one to three substituents independently selected from —F, —Cl, —Br, —I, —OH, —C₁-C₆ alkoxy independently optionally containing from one to three double or triple bonds, —NR⁹R¹⁰, -(CH₂)₁₋₁₀NR⁹R¹⁰, —C(═O)R¹¹, —S(O)ₙR¹¹, —C(═O)OR¹¹, —C(═O)NR⁹R¹⁰, —S(O)ₙNR⁹R¹⁰ -(C₃-C₁₂) cycloalkyl, -((4-12 membered) heterocycloalkyl), -(C₆-C₁₅) aryl, -((5-15 membered) heteroaryl), -((4-12 membered) heterocycloalkoxy), -(C₆-C₁₂) aryloxy and -((6-12 membered) heteroaryloxy).

In another embodiment, R⁷ is selected from —C₁-C₁₂ alkyl optionally containing from one to five double bonds and wherein each hydrogen is independently optionally replaced with a fluorine, -(C₃-C₁₂)cycloalkyl optionally substituted with from one to six fluorine and -((3-12 membered) heterocycloalkyl) optionally substituted with from one to six fluorine, wherein said alkyl, cycloalkyl and heterocycloalkyl are each optionally substituted with from one to three substitutents independently selected from —OH, —C₁-C₆ alkoxy independently optionally containing from one to three double or triple bonds, —NR⁹R¹⁰, -(CH₂)₁₋₆NR⁹R¹⁰, —C(═O)R¹¹, —C(═O)OR¹¹, —C(═O)NR⁹R¹⁰, —S(O)ₙNR⁹R¹⁰, -(C₆-C₁₄) aryl, -((5-15 membered) heteroaryl), -((4-12 membered) heterocycloalkoxy), -(C₆-C₁₂) aryloxy and -((6-12 membered) heteroaryloxy).

In another embodiment, the invention provides compounds of Formula I wherein R⁷ is selected from —C₁-C₁₂ alkyl optionally containing from one to five double bonds, -(C₃-C₁₂) cycloalkyl and -((3-12 membered) heterocycloalkyl), wherein said alkyl, cycloalkyl and heterocycloalkyl are each optionally substituted with from one to three substitutents independently selected from —OH, —C₁-C₆ alkoxy independently optionally containing from one to three double or triple bonds, —NR⁹R¹⁰, and -(CH₂)₁₋₆NR⁹R¹⁰.

In another embodiment, R⁷ is selected from —C₁-C₁₂ alkyl optionally containing from one to five double bonds, -(C₃-C₁₂) cycloalkyl and -(3-12 membered) heterocycloalkyl, wherein said alkyl, cycloalkyl and heterocycloalkyl are each optionally substituted with from one to three substitutents independently selected from —OH and —C₁-C₆ alkoxy independently optionally containing from one to three double or triple bonds.

In another embodiment, R⁷ is selected from —C₁-C₁₂ alkyl optionally containing from one to five double bonds and —C₃-C₁₅ cycloalkyl, wherein said alkyl and cycloalkyl are each optionally independently substituted with from one to three substitutents —NR⁹R¹⁰.

In another embodiment, R⁷ is -((3-12 membered) heterocycloalkyl), wherein said heterocycloalkyl is optionally substituted with from one to three substitutents independently selected from —OH, —C₁-C₆ alkyl independently optionally containing from one to three double or triple bonds, —C₁-C₆ alkoxy independently optionally containing from one to three double or triple bonds, -(C₆-C₁₀) aryl, and -(5-15 membered) heteroaryl.

The terms “halogen”, “halo”, and the like, as used herein, unless otherwise indicated, include F, Cl, Br, and I.

The term “alkyl”, as used herein, unless otherwise indicated, includes saturated monovalent hydrocarbon radicals having straight or branched moieties. Examples of alkyl groups include, but are not limited to, methyl, ethyl, n-propyl, isopropyl, and t-butyl.

The term “alkenyl”, as used herein, unless otherwise indicated, includes alkyl moieties having at least one carbon-carbon double bond wherein alkyl is as defined above. Examples of alkenyl include, but are not limited to, ethenyl and propenyl.

The term “alkynyl”, as used herein, unless otherwise indicated, includes alkyl moieties having at least one carbon-carbon triple bond wherein alkyl is as defined above. Examples of alkynyl groups include, but are not limited to, ethynyl and 2-propynyl.

The term “cycloalkyl”, as used herein, unless otherwise indicated, includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl. “Bicycloalkyl” and “tricycloalkyl” groups are non-aromatic saturated carbocyclic groups consisting of two or three rings respectively, wherein said rings share at least one carbon atom. For purposes of the present invention, and unless otherwise indicated, bicycloalkyl groups include spiro groups and fused ring groups. Examples of bicycloalkyl groups include, but are not limited to, bicyclo-[3.1.0]-hexyl, bicyclo-2.2.1]-hept-1-yl, norbornyl, spiro[4.5]decyl, spiro[4.4]nonyl, spiro[4.3]octyl, and spiro[4.2]heptyl. An example of a tricycloalkyl group is adamantanyl. Other cycloalkyl, bicycloalkyl, and tricycloalkyl groups are known in the art, and such groups are encompassed by the definitions “cycloalkyl”, “bicycloalkyl” and “tricycloalkyl” herein. “Cycloalkenyl”, “bicycloalkenyl”, and “tricycloalkenyl” refer to non-aromatic carbocyclic cycloalkyl, bicycloalkyl, and tricycloalkyl moieties as defined above, except comprising one or more carbon-carbon double bonds connecting carbon ring members (an “endocyclic” double bond) and/or one or more carbon-carbon double bonds connecting a carbon ring member and an adjacent non-ring carbon (an “exocyclic” double bond). Examples of cycloalkenyl groups include, but are not limited to, cyclopentenyl, cyclobutenyl, and cyclohexenyl, and a non-limiting example of a bicycloalkenyl group is norbornenyl. Cycloalkyl, cycloalkenyl, bicycloalkyl, and bicycloalkenyl groups also include groups that are substituted with one or more oxo moieties. Examples of such groups with oxo moieties are oxocyclopentyl, oxocyclobutyl, oxocyclopentenyl, and norcamphoryl. Other cycloalkenyl, bicycloalkenyl, and tricycloalkenyl groups are known in the art, and such groups are included within the definitions “cycloalkenyl”, “bicycloalkenyl” and “tricycloalkenyl” herein.

The term “aryl”, as used herein, unless otherwise indicated, includes an organic radical derived from an aromatic hydrocarbon by removal of one hydrogen, such as phenyl, naphthyl, indenyl, indanyl, and fluorenyl. “Aryl” encompasses fused ring groups wherein at least one ring is aromatic.

The terms “heterocyclic”, “heterocycloalkyl”, and like terms, as used herein, refer to non-aromatic cyclic groups containing one or more heteroatoms, prefereably from one to four heteroatoms, each selected from O, S and N. “Heterobicycloalkyl” groups are non-aromatic two-ringed cyclic groups, wherein said rings share one or two atoms, and wherein at least one of the rings contains a heteroatom (O, S, or N). Heterobicycloalkyl groups for purposes of the present invention, and unless otherwise indicated, include spiro groups and fused ring groups. “Heterotricycloalkyl” groups are non-aromatic three-ringed cyclic groups, wherein said rings are fused to one another or form a spiro group (in other words, at least two of said rings share one or two atoms and the third ring shares one or two atoms with at least one of said two rings). The heterocyclic (i.e. heterocycloalkyl, heterobicycloalkyl, and heterotricycloalkyl) groups of the compounds of the subject invention can include O, S(O)_{zero-2}, and/or N—R⁹ as heteroatoms, wherein R⁹ is as defined above, and wherein the subscript “zero-2” of S(O)_{zero-2} represents a group of integers consisting of zero, 1, and 2. Thus, S(O)_{zero-2} represents the group consisting of S, S(═O), and S(O)₂. In one embodiment, each ring in the heterobicycloalkyl or heterotricycloalkyl contains up to four heteroatoms (i.e. from zero to four heteroatoms, provided that at least one ring contains at least one heteroatom). The heterocyclic groups, including the heterobicyclic and heterotricyclic groups, of this invention can also include ring systems substituted with one or more oxo moieties. The heterocyclic groups, including the heterobicyclic and heterotricyclic groups, may comprise double or triple bonds, e.g. heterocycloalkenyl, heterobicycloalkenyl, and heterotricycloalkenyl. Examples of non-aromatic heterocyclic groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

“Heteroaryl”, as used herein, refers to aromatic groups containing one or more heteroatoms (O, S, or N), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, 1,2,3,4-tetrahydroguinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, 1,2,4-trizainyl, 1,3,5-triazinyl, isoindolyl, 1-oxoisoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The foregoing groups, as derived from the compounds listed above, may be C-attached or N-attached where such is possible. For instance, a group derived from pyrrole may be pyrrol-1-yl (N-attached) or pyrrol-3-yl (C-attached). The terms referring to the groups also encompass all possible tautomers.

Compounds of Formula I may have optical centers and therefore may occur in different enantiomeric, diastereomeric and meso configurations. The invention includes all enantiomers, diastereomers, and other stereoisomers of such compounds of Formula I, as well as racemic and other mixtures thereof. The invention also includes all tautomers of Formula I. When the compounds of Formula I of the present invention contain one optical center, the “S” enantiomer is preferred.

The subject invention also includes isotopically-labeled compounds of Formula I, which are identical to those recited in Formula I, but for the fact that one or more atoms are replaced by an atom having an atomic mass or mass number different from the atomic mass or mass number most abundant in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I. Compounds of Formula I of the present invention and pharmaceutically acceptable salts, complexes and derivatives of said compounds that contain the aforementioned isotopes and/or other isotopes of other atoms are within the scope of this invention. Isotopically-labeled compounds of Formula I, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula I of this invention can generally be prepared by substituting a readily available isotopically labeled reagent for a non-isotopically labeled reagent in the preparation of said compounds.

Salts of compounds of Formula I can be obtained by forming salts with any acidic or basic group present on a compound of Formula I. Examples of pharmaceutically acceptable salts of the compounds of Formula I are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, maleic acid, di-p-toluoyl tartaric acid, acetic acid, sulfuric acid, hydroiodic acid, mandelic acid, sodium, potassium, magnesium, calcium, and lithium.

The subject invention also includes all prodrugs of compounds of Formula I. A prodrug is a compound that may not possess the desired pharmacological activity per se, but can be administered, for example parenterally or orally, to a mammal, thereafter being metabolized in the mammal's body to form a compound that does have the desired pharmacological activity. For example, a prodrug of a compound of Formula I is metabolized, after administration to a mammal, to a compound of Formula I. Examples of prodrugs of Formula I include compound of Formula I wherein a hydroxy moiety is replaced with a moiety selected from —CH(OC(═O)R^{2a})R^{1a} and —CH(OC(═O)OR^{2a})R^{1a}, wherein R^{2a} is selected from —C₁-C₄ alkyl, —C(OH)(C₁-C₄ alkyl), —CH(OH)((C₅-C₆) aryl), —CH(OH)((₅-₆ membered) heteroaryl), —CH(OH)(C₅-C₆ cycloalkyl), —CH(OH)(C₅-C₆ cycloalkenyl), and —CH(OH)((₅-₆ membered) heterocycloalkyl). Further, it will be appreciated by those skilled in the art that certain protected derivatives of compounds of Formula I, which may be made prior to a final deprotection stage, may, in certain instances, be administered to a mammal and thereafter metabolized in the mammal's body to form compounds of the invention which are pharmacologically active. Such derivatives are therefore also “prodrugs” of compounds of Formula I and are part of the present invention.

Preferred embodiments of this invention include the following compounds of Formula I, and all pharmaceutically acceptable salts thereof, complexes thereof, and derivatives thereof which convert into a pharmaceutically active compound upon administration:

2-[2-(3,5-difluoro-phenyl)-acetylamino]-N-(4-phenyl-thiazol-2-yl)-propionamide;

2-{2-[2-(3,5-difluoro-phenyl)-acetylamino]-propionylamino}-4-phenyl-thiazole-5-carboxylic acid ethyl ester;

(2-{2-[2-(3,5-difluoro-phenyl)-acetylamino]-pentanoylamino}-thiazol-4-yl)-acetic acid ethyl ester;

2-[2-(3,5-difluoro-phenyl)-acetylamino]-pentanoic acid [5-(4-nitro-benzenesulfonyl)-thiazol-2-yl]-amide;

2-[2-(3,5-difluoro-phenyl)-acetylamino]-pentanoic acid [5-(4-hydroxyamino-benzenesulfonyl)-thiazol-2-yl]-amide;

2-[2-(3,5-difluoro-phenyl)-acetylamino]-pentanoic acid [5-(4-amino-benzenesulfonyl)-thiazol-2-yl]-amide;

N-[5-(5-bromo-thiophen-2-yl)-thiazol-2-yl]-2-[2-(3,5-difluoro-phenyl)-acetylamino]-butyramide;

2-[2-(3,5-difluoro-phenyl)-acetylamino]-pentanoic acid [5-(4-benzylamino-benzenesulfonyl)-thiazol-2-yl]-amide;

2-[2-(3,5-difluoro-phenyl)-acetylamino]-pentanoic acid benzothiazol-2-ylamide;

2-[2-(3,5-difluoro-phenyl)-acetylamino]-pentanoic acid (5-methyl-thiazol-2-yl)-amide;

2-[2-(3,5-difluoro-phenyl)-acetylamino]-pentanoic acid (4,5-dimethyl-thiazol-2-yl)-amide;

2-[2-(3,5-difluoro-phenyl)-acetylamino]-pentanoic acid (5-nitro-thiazol-2-yl)-amide;

2-[2-(3,5-difluoro-phenyl)-acetylamino]-pentanoic acid thiazol-2-ylamide;

2-[2-(3,5-difluoro-phenyl)-acetylamino]-pentanoic acid (5,6-dihydro-4H-cyclopentathiazol-2-yl)-amide;

2-[2-(3,5-difluoro-phenyl)-acetylamino]-pentanoic acid (5-chloro-thiazol-2-yl)-amide;

2-[2-(3,5-difluoro-phenyl)-acetylamino]-pentanoic acid (4-methyl-thiazol-2-yl)-amide;

(2-{2-[2-(3,5-difluoro-phenyl)-acetylamino]-pentanoylamino}-thiazol-4-yl)-acetic acid;

2-[2-(3,5-difluoro-phenyl)-acetylamino]-pentanoic acid (5-amino-thiazol-2-yl)-amide;

2-[2-(3,5-difluoro-phenyl)-acetylamino]-pentanoic acid [5-(4-chloro-benzenesulfonyl)-thiazol-2-yl]-amide;

2-[2-(3,5-difluoro-phenyl)-acetylamino]-N-[5-(5-methoxy-1,5-dimethyl-hexyl)-thiazol-2-yl]-butyramide;

2-[2-(3,5-difluoro-phenyl)-acetylamino]-N-(5,6,7,8-tetrahydro-4H-cycloheptathiazol-2-yl)-butyramide;

N-(4-cyclopentyl-thiazol-2-yl)-2-[2-(3,5-difluoro-phenyl)-acetylamino]-butyramide;

2-[2-(3,5-difluoro-phenyl)-acetylamino]-N-(6-methyl-4,5,6,7-tetrahydro-benzothiazol-2-yl)-butyramide;

2-[2-(3,5-difluoro-phenyl)-acetylamino]-N-(5-methylsulfanyl-thiazol-2-yl)-butyramide;

2-[2-(3,5-difluoro-phenyl)-acetylamino]-N-(5-isopropyl-thiazol-2-yl)-butyramide;

2-[2-(3,5-difluoro-phenyl)-acetylamino]-pentanoic acid {4-[(butyl-ethyl-carbamoyl)-methyl]-thiazol-2-yl}-amide;

2-[2-(3,5-difluoro-phenyl)-acetylamino]-pentanoic acid [4-(benzylcarbamoyl-methyl)-thiazol-2-yl]-amide;

2-[2-(3,5-difluoro-phenyl)-acetylamino]-pentanoic acid (5-bromo-thiazol-2-yl)-amide;

2-[2-(3,5-difluoro-phenyl)-acetylamino]-N-(4-phenyl-thiazol-2-yl)-butyramide;

2-[2-(3,5-difluoro-phenyl)-acetylamino]-N-(4,5-diphenyl-thiazol-2-yl)-butyramide;

N-(5-acetyl-thiazol-2-yl)-2-[2-(3,5-difluoro-phenyl)-acetylamino]-butyramide;

2-[2-(3,5-difluoro-phenyl)-acetylamino]-pentanoic acid (4-ethylcarbamoylmethyl-thiazol-2-yl)-amide;

N-(5-sec-butyl-thiazol-2-yl)-2-[2-(3,5-difluoro-phenyl)-acetylamino]-butyramide;

2-[2-(3,5-difluoro-phenyl)-acetylamino]-N-(6-methyl-benzothiazol-2-yl)-butyramide;

2-[2-(3,5-difluoro-phenyl)-acetylamino]-N-(6-methoxy-benzothiazol-2-yl)-butyramide;

N-(6-chloro-benzothiazol-2-yl)-2-[2-(3,5-difluoro-phenyl)-acetylamino]-butyramide;

N-(4-chloro-benzothiazol-2-yl)-2-[2-(3,5-difluoro-phenyl)-acetylamino]-butyramide;

2-[2-(3,5-difluoro-phenyl)-acetylamino]-pentanoic acid {4-[(cyclopropylmethyl-carbamoyl)-methyl]-thiazol-2-yl}-amide;

3,7-dimethyl-oct-6-enoic acid [1-(5-methyl-thiazol-2-ylcarbamoyl)-butyl]-amide;

2-(2-cyclohexyl-2-hydroxy-acetylamino)-pentanoic acid (5-methyl-thiazol-2-yl)-amide;

2-[2-(3,5-difluoro-phenyl)-acetylamino]-N-(4,5,6,7-tetrahydro-benzothiazol-2-yl)-butyramide;

2-(2-{2-[2-(3,5-difluoro-phenyl)-acetylamino]-butyrylamino}-thiazol-4-yl)-2-methyl-propionic acid ethyl ester;

2-[2-(3,5-difluoro-phenyl)-acetylamino]-N-[6-(piperidine-1-sulfonyl)-benzothiazol-2-yl]-butyramide;

2-[2-(3,5-difluoro-phenyl)-2-hydroxy-acetylamino]-pentanoic acid (5-methyl-thiazol-2-yl)-amide;

2-[2-(3,5-difluoro-phenyl)-acetylamino]-N-[5-(4-fluoro-phenyl)-thiazol-2-yl]-butyramide;

(2-{2-[2-(3,5-difluoro-phenyl)-acetylamino]-butyrylamino}-thiazol-4-yl)-methoxyimino-acetic acid ethyl ester;

2-[2-(5-bromo-pyridin-3-yl)-acetylamino]-pentanoic acid (5-methyl-thiazol-2-yl)-amide;

2-[2-(3-phenoxy-phenyl)-acetylamino]-pentanoic acid (5-butyl-thiazol-2-yl)-amide;

2-(2-hydroxy-3-methyl-butyrylamino)-pentanoic acid (5-isopropyl-thiazol-2-yl)-amide;

2-[2-(3,5-difluoro-phenyl)-acetylamino]-pentanoic acid (5-isopropyl-thiazol-2-yl)-amide;

4-methyl-2-{2-[2-(3-phenoxy-phenyl)-acetylamino]-pentanoylamino}-thiazole-5-carboxylic acid dimethylamide;

2-[2-(5-bromo-pyridin-3-yl)-acetylamino]-pentanoic acid (5-isopropyl-thiazol-2-yl)-amide;

3,7-dimethyl-oct-6-enoic acid [1-(5-isopropyl-thiazol-2-ylcarbamoyl)-butyl]-amide;

2-(2-hydroxy-3-methyl-butyrylamino)-pentanoic acid (5-isopropyl-thiazol-2-yl)-amide;

2-hydroxy-N-[2-(2-hydroxy-3-methyl-butyrylamino)-pentanoyl]-N-(5-isopropyl-thiazol-2-yl)-3-methyl-butyramide;

3,7-dimethyl-oct-6-enoic acid [1-(5-isopropyl-thiazol-2-ylcarbamoyl)-butyl]-amide;

2-{2-[2-(3,5-difluoro-phenyl)-acetylamino]-pentanoylamino}-4-ethoxymethyl-thiazole-5-carboxylic acid ethyl ester;

2-{2-[2-(3,5-difluoro-phenyl)-acetylamino]-pentanoylamino}-thiazole-5-carboxylic acid amide;

2-[2-(3,5-difluoro-phenyl)-acetylamino]-pentanoic acid {5-[(4-hydroxy-4-phenyl-piperidin-1-yl)-acetyl]-thiazol-2-yl}-amide;

2-[2-(3,5-difluoro-phenyl)-acetylamino]-pentanoic acid [5-(methyl-phenyl-amino)-thiazol-2-yl]-amide;

2-{2-[2-(3,5-difluoro-phenyl)-acetylamino]-pentanoylamino}-4-methyl-thiazole-5-carboxylic acid (4-chloro-phenyl)-amide;

2-{2-[2-(3,5-difluoro-phenyl)-acetylamino]-pentanoylamino}-thiazole-5-carboxylic acid methyl ester; and

2-[2-(3,5-difluoro-phenyl)-acetylamino]-pentanoic acid (5-acetyl-thiazol-2-yl)-amide.

Other preferred embodiments of this invention include the following compounds of Formula I, and all pharmaceutically acceptable salts thereof, complexes thereof, and derivatives thereof which convert into a pharmaceutically active compound upon administration:

(2-{2-[2-(3,5-Difluoro-phenyl)-acetylamino]-pentanoylamino}-thiazol-4-yl)-acetic acid ethyl ester;

(2-{2-[2-(3,5-Difluoro-phenyl)-acetylamino]-butyrylamino}-thiazol-4-yl)-methoxyimino-acetic acid ethyl ester;

2-{2-[2-(3,5-Difluoro-phenyl)-acetylamino]-pentanoylamino}-thiazole-5-carboxylic acid methyl ester;

2-[2-(3,5-Difluoro-phenyl)-acetylamino]-pentanoic acid (5-acetyl-thiazol-2-yl)-amide;

2-[2-(3,5-Difluoro-phenyl)-2-hydroxy-acetylamino]-pentanoic acid [5-(5-methoxy-1,5-dimethyl-hexyl)-thiazol-2-yl]-amide;

2-(2-Hydroxy-3,3-dimethyl-butyrylamino)-pentanoic acid (5-isopropyl-thiazol-2-yl)-amide;

2-[2-(3,5-Difluoro-phenyl)-2-hydroxy-acetylamino]-pentanoic acid (5-methyl-thiazol-2-yl)-amide;

2-[2-(3,5-Difluoro-phenyl)-2-hydroxy-acetylamino]-pentanoic acid (5-methyl-thiazol-2-yl)-amide;

Hydroxy-phenyl-acetic acid [1-(5-isopropyl-thiazol-2-ylcarbamoyl)-butylcarbamoyl]-phenyl-methyl ester;

2-(2-Hydroxy-2-phenyl-acetylamino)-pentanoic acid (5-isopropyl-thiazol-2-yl)-amide;

2-(2-Hydroxy-3-methyl-butyrylamino)-pentanoic acid (5-methyl-thiazol-2-yl)-amide;

2-(2-Hydroxy-2-phenyl-acetylamino)-pentanoic acid (4,5-dimethyl-thiazol-2-yl)-amide;

2-[2-(3,5-Difluoro-phenyl)-acetylamino]-N-[5-(1,5-dimethyl-hex-4-enyl)-thiazol-2-yl]-butyramide;

2-[2-(3,5-Difluoro-phenyl)-acetylamino]-hexanoic acid (5-isopropyl-thiazol-2-yl)-amide;

2-[2-(3,5-Difluoro-phenyl)-acetylamino]-N-[5-(5-hydroxy-1,5-dimethyl-hexyl)-thiazol-2-yl]-propionamide;

2-[2-(3,5-Difluoro-phenyl)-acetylamino]-N-[5-(5-methoxy-1,5-dimethyl-hexyl)-thiazol-2-yl]-propionamide;

2-[2-(3,5-Difluoro-phenyl)-2-hydroxy-acetylamino]-N-[5-(5-hydroxy-1,5-dimethyl-hexyl)-thiazol-2-yl]-propionamide;

2-[2-(3,5-Difluoro-phenyl)-2-hydroxy-acetylamino]-N-[5-(5-methoxy-1,5-dimethyl-hexyl)-thiazol-2-yl]-propionamide;

2-Hydroxy-N-{1-[5-(5-hydroxy-1,5-dimethyl-hexyl)-thiazol-2-ylcarbamoyl]-ethyl}-3-methyl-butyramide;

2-Hydroxy-N-{1-[5-(5-methoxy-1,5-dimethyl-hexyl)-thiazol-2-ylcarbamoyl]-ethyl}-3-methyl-butyramide;

2-Hydroxy-N-{1-[5-(5-hydroxy-1,5-dimethyl-hexyl)-thiazol-2-ylcarbamoyl]-ethyl}-3,3-dimethyl-butyramide;

2-Hydroxy-N-{1-[5-(5-methoxy-1,5-dimethyl-hexyl)-thiazol-2-ylcarbamoyl]-ethyl}-3,3-dimethyl-butyramide;

N-[5-(5-Hydroxy-1,5-dimethyl-hexyl)-thiazol-2-yl]-2-(2-hydroxy-2-phenyl-acetylamino)-propionamide;

2-(2-Hydroxy-2-phenyl-acetylamino)-N-[5-(5-methoxy-1,5-dimethyl-hexyl)-thiazol-2-yl]-propionamide;

N-[5-(5-Hydroxy-1,5-dimethyl-hexyl)-thiazol-2-yl]-2-(2-oxo-2-thiophen-2-yl-acetylamino)-propionamide;

N-[5-(5-Methoxy-1,5-dimethyl-hexyl)-thiazol-2-yl]-2-(2-oxo-2-thiophen-2-yl-acetylamino)-propionamide;

2-[2-(5-Bromo-pyridin-3-yl)-acetylamino]-N-[5-(5-hydroxy-1,5-dimethyl-hexyl)-thiazol-2-yl]-propionamide;

2-[2-(5-Bromo-pyridin-3-yl)-acetylamino]-N-[5-(5-methoxy-1,5-dimethyl-hexyl)-thiazol-2-yl]-propionamide;

2-[2-(3,5-Difluoro-phenyl)-acetylamino]-pentanoic acid [5-(1,3,3-trimethyl-butyl)-thiazol-2-yl]-amide;

2-(2-Hydroxy-3,3-dimethyl-butyrylamino)-pentanoic acid [5-(5-methoxy-1,5-dimethyl-hexyl)-thiazol-2-yl]-amide;

2-(2-Hydroxy-3-methyl-butyrylamino)-pentanoic acid [5-(5-methoxy-1,5-dimethyl-hexyl)-thiazol-2-yl]-amide;

2-[2-(3,5-Difluoro-phenyl)-3-hydroxy-3-methyl-butyrylamino]-pentanoic acid thiazol-2-ylamide;

2-[2-(3,5-Difluoro-phenyl)-acetylamino]-pentanoic acid [5-(1-hydroxy-1-methyl-ethyl)-thiazol-2-yl]-amide;

2-[2-(2-Hydroxy-3,3-dimethyl-butyrylamino)-butyrylamino]-4-trifluoromethyl-thiazole-5-carboxylic acid ethyl ester;

2-[2-(3,5-Difluoro-phenyl)-acetylamino]-pentanoic acid benzyl-thiazol-2-yl-amide;

2-(2-Oxo-2-thiophen-2-yl-acetylamino)-pentanoic acid [5-(5-methoxy-1,5-dimethyl-hexyl)-thiazol-2-yl]-amide;

2-[2-(3,5-Difluoro-phenyl)-acetylamino]-N-[5-(5-methoxy-1,5-dimethyl-hexyl)-thiazol-2-yl]-butyramide;

2-(2-Oxo-2-thiophen-2-yl-acetylamino)-pentanoic acid [5-(5-hydroxy-1,5-dimethyl-hexyl)-thiazol-2-yl]-amide;

2-(2-Oxo-2-thiophen-2-yl-acetylamino)-pentanoic acid [5-(1,5-dimethyl-hex-4-enyl)-thiazol-2-yl]-amide;

2-(3,3-Dimethyl-2-oxo-butyrylamino)-pentanoic acid (5-isopropyl-thiazol-2-yl)-amide;

2-[2-(3,5-Difluoro-phenyl)-acetylamino]-pentanoic acid [5-(1-ethyl-1-hydroxy-propyl)-thiazol-2-yl]-amide;

2-(2-Hydroxy-2-phenyl-acetylamino)-pentanoic acid [5-(5-hydroxy-1,5-dimethyl-hexyl)-thiazol-2-yl]-amide;

2-[2-(3,5-Difluoro-phenyl)-acetylamino]-N-[5-(5-methoxy-1,5-dimethyl-hexyl)-thiazol-2-yl]-butyramide;

2-(2-Hydroxy-2-phenyl-acetylamino)-pentanoic acid [5-(5-methoxy-1,5-dimethyl-hexyl)-thiazol-2-yl]-amide;

2-Hydroxy-N-[1-(5-isopropyl-thiazol-2-ylcarbamoyl)-ethyl]-3,3-dimethyl-butyramide;

2-(2-Hydroxy-2-phenyl-acetylamino)-N-(5-isopropyl-thiazol-2-yl)-butyramide;

2-(2-Hydroxy-2-phenyl-acetylamino)-N-(5-isopropyl-thiazol-2-yl)-propionamide;

2-Hydroxy-N-[1-(5-isopropyl-thiazol-2-ylcarbamoyl)-ethyl]-3-methyl-butyramide;

2-Hydroxy-N-[1-(5-isopropyl-thiazol-2-ylcarbamoyl)-propyl]-3-methyl-butyramide;

2-Hydroxy-3,3-dimethyl-butyric acid 1-[1-(5-isopropyl-thiazol-2-ylcarbamoyl)-ethylcarbamoyl]-2,2-dimethyl-propyl ester;

Hydroxy-phenyl-acetic acid [1-(5-isopropyl-thiazol-2-ylcarbamoyl)-propylcarbamoyl]-phenyl-methyl ester;

2-Hydroxy-3-methyl-butyric acid 1-[1-(5-isopropyl-thiazol-2-ylcarbamoyl)-propylcarbamoyl]-2-methyl-propyl ester;

2-Hydroxy-3-methyl-butyric acid 1-{1-[1-(5-isopropyl-thiazol-2-ylcarbamoyl)-propylcarbamoyl]-2-methyl-propoxycarbonyl}-2-methyl-propyl ester;

2-[2-(5-Bromo-pyridin-3-yl)-2-hydroxy-acetylamino]-N-[5-(5-methoxy-1,5-dimethyl-hexyl)-thiazol-2-yl]-butyramide;

2-[2-(5-Bromo-pyridin-3-yl)-acetylamino]-N-(5-isopropyl-thiazol-2-yl)-butyramide;

Hydroxy-phenyl-acetic acid [1-(5-isopropyl-thiazol-2-ylcarbamoyl)-ethylcarbamoyl]-phenyl-methyl ester;

2-Hydroxy-3-methyl-butyric acid 1-[1-(5-isopropyl-thiazol-2-ylcarbamoyl)-ethylcarbamoyl]-2-methyl-propyl ester;

2-Hydroxy-N-[1-(5-isopropyl-thiazol-2-ylcarbamoyl)-propyl]-3,3-dimethyl-butyramide;

2-(2-Hydroxy-3,3-dimethyl-butyrylamino)-pentanoic acid (5-isopropenyl-thiazol-2-yl)-amide;

2-(2-Hydroxy-3,3-dimethyl-butyrylamino)-pentanoic acid [5-(1-hydroxy-1-methyl-ethyl)-thiazol-2-yl]-amide;

2-[2-(5-Bromo-pyridin-3-yl)-acetylamino]-N-(5-isopropyl-thiazol-2-yl)-propionamide;

2-(3,5-Difluoro-phenyl)-3-hydroxy-3-methyl-pentanoic acid [1-(thiazol-2-ylcarbamoyl)-butyl]-amide;

2-(2-Hydroxy-3,3-dimethyl-butyrylamino)-pentanoic acid [5-(1,3,3-trimethyl-butyl)-thiazol-2-yl]-amide;

1-(3,5-Difluoro-phenyl)-cyclopentanecarboxylic acid [1-(5-methyl-thiazol-2-ylcarbamoyl)-butyl]-amide;

2-[2-(3,5-Difluoro-phenyl)-acetylamino]-N-[5-(1,5-dimethyl-hex-4-enyl)-thiazol-2-yl]-propionamide;

2-[2-(3,5-Difluoro-phenyl)-acetylamino]-pentanoic acid [5-(1-butylamino-ethyl)-thiazol-2-yl]-amide;

2-[2-(3,5-Difluoro-phenyl)-acetylamino]-pentanoic acid [5-(1-butylamino-ethyl)-thiazol-2-yl]-amide;

2-[2-(3,5-Difluoro-phenyl)-acetylamino]-pentanoic acid [5-(1-ethyl-propyl)-thiazol-2-yl]-amide;

2-(2-Hydroxy-3,3-dimethyl-butyrylamino)-pentanoic acid [5-(1,5-dimethyl-hex-4-enyl)-thiazol-2-yl]-amide;

2-(2-Amino-3,3-dimethyl-butyrylamino)-pentanoic acid (5-isopropyl-thiazol-2-yl)-amide;

2-(2-Hydroxy-3,3-dimethyl-butyrylamino)-pentanoic acid [5-(5-methoxy-1,5-dimethyl-hexyl)-thiazol-2-yl]-amide;

2-(2-Hydroxy-3,3-dimethyl-butyrylamino)-pentanoic acid [5-(5-methoxy-1,5-dimethyl-hexyl)-thiazol-2-yl]-amide;

2-(2-Hydroxy-3,3-dimethyl-butyrylamino)-pentanoic acid [5-(5-hydroxy-1,5-dimethyl-hexyl)-thiazol-2-yl]-amide;

2-(2-Hydroxy-3,3-dimethyl-butyrylamino)-pentanoic acid [5-(5-hydroxy-1,5-dimethyl-hexyl)-thiazol-2-yl]-amide;

2-(2-Hydroxy-3-methyl-butyrylamino)-pentanoic acid [5-(5-methoxy-1,5-dimethyl-hexyl)-thiazol-2-yl]-amide;

2-(2-Hydroxy-3-methyl-butyrylamino)-pentanoic acid [5-(5-methoxy-1,5-dimethyl-hexyl)-thiazol-2-yl]-amide;

2-(2-Hydroxy-3,3-dimethyl-butyrylamino)-pentanoic acid (5-acetyl-thiazol-2-yl)-amide;

2-(2-Hydroxy-3,3-dimethyl-butyrylamino)-pentanoic acid (5-acetyl-thiazol-2-yl)-amide;

2-[2-(3,5-Difluoro-phenyl)-acetylamino]-pentanoic acid [5-(1-propyl-butyl)-thiazol-2-yl]-amide;

2-[2-(3,5-Difluoro-phenyl)-acetylamino]-pentanoic acid [5-(1-propyl-butyl)-thiazol-2-yl]-amide;

2-[2-(3,5-Difluoro-phenyl)-acetylamino]-pentanoic acid [5-(1-ethyl-3-methyl-butyl)-thiazol-2-yl]-amide;

2-[2-(3,5-Difluoro-phenyl)-acetylamino]-pentanoic acid [5-(1-ethyl-1-hydroxy-propyl)-thiazol-2-yl]-amide;

2-(2-Hydroxy-3,3-dimethyl-butyrylamino)-pentanoic acid [5-(1-ethyl-1-hydroxy-propyl)-thiazol-2-yl]-amide;

2-[2-(3,5-Difluoro-phenyl)-acetylamino]-pentanoic acid [5-(1-hydroxy-ethyl)-thiazol-2-yl]-amide;

2-[2-(3,5-Difluoro-phenyl)-acetylamino]-pentanoic acid (5-acetyl-4-methyl-thiazol-2-yl)-amide;

2-[2-(3,5-Difluoro-phenyl)-acetylamino]-pentanoic acid [5-(1-butylamino-ethyl)-4-methyl-thiazol-2-yl]-amide;

2-[2-(3,5-Difluoro-phenyl)-acetylamino]-pentanoic acid [4-methyl-5-(1-propylamino-ethyl)-thiazol-2-yl]-amide;

2-[2-(3,5-Difluoro-phenyl)-acetylamino]-pentanoic acid [5-(3,3-dimethyl-cyclohexyl)-thiazol-2-yl]-amide;

2-[2-(3,5-Difluoro-phenyl)-acetylamino]-pentanoic acid (5-ethyl-thiazol-2-yl)-amide;

2-[2-(3,5-Difluoro-phenyl)-acetylamino]-pentanoic acid [5-(1-benzyl-4-hydroxy-piperidin-4-yl)-thiazol-2-yl]-amide;

2-[2-(3,5-Difluoro-phenyl)-acetylamino]-pentanoic acid (5-formyl-thiazol-2-yl)-amide;

2-[2-(3,5-Difluoro-phenyl)-acetylamino]-pentanoic acid (5-ethylsulfanyl-thiazol-2-yl)-amide;

2-[2-(3,5-Difluoro-phenyl)-acetylamino]-pentanoic acid (8H-3-thia-1-aza-cyclopenta[a]inden-2-yl)-amide;

2-[2-(3,5-Difluoro-phenyl)-acetylamino]-pentanoic acid [4-phenyl-5-(piperidine-1-carbonyl)-thiazol-2-yl]-amide;

(2-{2-[2-(3,5-Difluoro-phenyl)-acetylamino]-pentanoylamino}-thiazol-5-ylmethylsulfanyl)-acetic acid ethyl ester;

2-[2-(3,5-Difluoro-phenyl)-acetylamino]-pentanoic acid [5-(1-hydroxy-ethyl)-4-methyl-thiazol-2-yl]-amide;

2-[2-(3,5-Difluoro-phenyl)-acetylamino]-pentanoic acid [5-(1-hydroxy-ethyl)-4-methyl-thiazol-2-yl]-amide;

2-[2-(3,5-Difluoro-phenyl)-acetylamino]-pentanoic acid [5-(1-ethyl-propenyl)-thiazol-2-yl]-amide;

2-(2-Hydroxy-2-phenyl-acetylamino)-pentanoic acid [5-(1-ethyl-1-hydroxy-propyl)-thiazol-2-yl]-amide;

2-(2-Hydroxy-2-phenyl-acetylamino)-pentanoic acid [5-(1-ethyl-1-hydroxy-propyl)-thiazol-2-yl]-amide;

2-[2-(3,5-Difluoro-phenyl)-acetylamino]-pentanoic acid {5-[1-(2-methoxy-ethylamino)-ethyl]-4-methyl-thiazol-2-yl}-amide;

2-[2-(3,5-Difluoro-phenyl)-acetylamino]-pentanoic acid [4-methyl-5-(1-pyrrolidin-1-yl-ethyl)-thiazol-2-yl]-amide;

2-(2-Hydroxy-3,3-dimethyl-butyrylamino)-pentanoic acid [5-(1-ethyl-propyl)-thiazol-2-yl]-amide;

2-[2-(3-Phenoxy-phenyl)-acetylamino]-pentanoic acid [5-(1-ethyl-propyl)-thiazol-2-yl]-amide;

2-(2-Hydroxy-3-methyl-butyrylamino)-pentanoic acid [5-(1-ethyl-propyl)-thiazol-2-yl]-amide;

2-[2-(5-Bromo-pyridin-3-yl)-acetylamino]-pentanoic acid [5-(1-ethyl-propyl)-thiazol-2-yl]-amide;

2-[2-(3,5-Difluoro-phenyl)-acetylamino]-pentanoic acid [5-(1,3-dimethyl-but-1-enyl)-thiazol-2-yl]-amide;

2-[2-(3,5-Difluoro-phenyl)-acetylamino]-pentanoic acid [5-(1-isobutyl-vinyl)-thiazol-2-yl]-amide;

2-[2-(3,5-Difluoro-phenyl)-acetylamino]-pentanoic acid {5-[(1-benzyl-piperidin-4-ylamino)-methyl]-thiazol-2-yl}-amide;

2-[2-(3,5-Difluoro-phenyl)-2-hydroxy-acetylamino]-pentanoic acid [5-(1-ethyl-propyl)-thiazol-2-yl]-amide;

2-[2-(3,5-Difluoro-phenyl)-2-hydroxy-acetylamino]-pentanoic acid [5-(1-ethyl-propyl)-thiazol-2-yl]-amide;

2-[2-(3,5-Difluoro-phenyl)-acetylamino]-pentanoic acid [4-methyl-5-(1-methylamino-ethyl)-thiazol-2-yl]-amide;

2-[2-(3,5-Difluoro-phenyl)-acetylamino]-pentanoic acid [5-(1-ethylamino-ethyl)-4-methyl-thiazol-2-yl]-amide;

2-[2-(3,5-Difluoro-phenyl)-acetylamino]-pentanoic acid [5-(1-isopropylamino-ethyl)-4-methyl-thiazol-2-yl]-amide;

2-[2-(3,5-Difluoro-phenyl)-acetylamino]-pentanoic acid {5-[1-(2-hydroxy-ethylamino)-ethyl]-4-methyl-thiazol-2-yl}-amide;

2-[2-(3,5-Difluoro-phenyl)-acetylamino]-pentanoic acid [4-methyl-5-(1-morpholin-4-yl-ethyl)-thiazol-2-yl]-amide;

2-[2-(3,5-Difluoro-phenyl)-acetylamino]-pentanoic acid {4-methyl-5-[1-(4-methyl-piperazin-1-yl)-ethyl]-thiazol-2-yl}-amide;

2-[2-(3,5-Difluoro-phenyl)-acetylamino]-N-[5-(1-ethyl-propyl)-thiazol-2-yl]-propionamide;

N-{1-[5-(1-Ethyl-propyl)-thiazol-2-ylcarbamoyl]-ethyl}-2-hydroxy-3,3-dimethyl-butyramide;

2-(2-Hydroxy-2-phenyl-acetylamino)-pentanoic acid [5-(1-ethyl-propyl)-thiazol-2-yl]-amide;

2-[2-(3,5-Difluoro-phenyl)-acetylamino]-pentanoic acid (5-{[ethyl-(2-hydroxy-ethyl)-amino]-methyl}-thiazol-2-yl)-amide;

2-[2-(3,5-Difluoro-phenyl)-acetylamino]-pentanoic acid {5-[1-(3,3-dimethyl-butylamino)-ethyl]-4-methyl-thiazol-2-yl}-amide;

2-[2-(3,5-Difluoro-phenyl)-acetylamino]-pentanoic acid [5-(1-isobutylamino-ethyl)-4-methyl-thiazol-2-yl]-amide;

2-[2-(3,5-Difluoro-phenyl)-acetylamino]-pentanoic acid {4-methyl-5-[1-(3-methyl-butylamino)-ethyl]-thiazol-2-yl}-amide;

2-[2-(3,5-Difluoro-phenyl)-acetylamino]-pentanoic acid (5-hydroxymethyl-thiazol-2-yl)-amide;

2-[2-(3,5-Difluoro-phenyl)-acetylamino]-pentanoic acid (5-morpholin-4-ylmethyl-thiazol-2-yl)-amide;

2-[2-(3,5-Difluoro-phenyl)-acetylamino]-pentanoic acid {5-[(butyl-ethyl-amino)-methyl]-thiazol-2-yl}-amide;

2-[2-(3,5-Difluoro-phenyl)-acetylamino]-pentanoic acid (5-trimethylsilanyl-thiazol-2-yl)-amide;

2-(2-Hydroxy-3,3-dimethyl-butyrylamino)-pentanoic acid (5-acetyl-4-methyl-thiazol-2-yl)-amide;

2-(2-Hydroxy-3-methyl-butyrylamino)-pentanoic acid (5-acetyl-4-methyl-thiazol-2-yl)-amide;

2-(2-Hydroxy-2-phenyl-acetylamino)-pentanoic acid (5-acetyl-4-methyl-thiazol-2-yl)-amide;

2-(2-Hydroxy-3,3-dimethyl-butyrylamino)-pentanoic acid {5-[1-(5-acetyl-4-methyl-thiazol-2-ylimino)-ethyl]-4-methyl-thiazol-2-yl}-amide;

2-(2-Hydroxy-2-phenyl-acetylamino)-pentanoic acid (5-trifluoroacetyl-thiazol-2-yl)-amide;

2-[2-(3,5-Difluoro-phenyl)-acetylamino]-pentanoic acid {5-[(1-ethyl-propylamino)-methyl]-thiazol-2-yl}-amide;

N-[5-(1-Ethyl-propyl)-thiazol-2-yl]-2-(2-hydroxy-2-phenyl-acetylamino)-propionamide;

N-[5-(1-Ethyl-propyl)-thiazol-2-yl]-2-(2-hydroxy-2-phenyl-acetylamino)-butyramide;

2-[2-(3,5-Difluoro-phenyl)-acetylamino]-pentanoic acid (5-ethylaminomethyl-thiazol-2-yl)-amide;

2-[2-(3,5-Difluoro-phenyl)-acetylamino]-pentanoic acid (5-dimethylaminomethyl-thiazol-2-yl)-amide;

2-[2-(3,5-Difluoro-phenyl)-acetylamino]-pentanoic acid [5-(isopropylamino-methyl)-thiazol-2-yl]-amide;

2-(2-Hydroxy-2-phenyl-acetylamino)-pentanoic acid [5-(2,2,2-trifluoro-1-hydroxy-ethyl)-thiazol-2-yl]-amide;

2-[2-(3,5-Difluoro-phenyl)-acetylamino]-pentanoic acid (5-aminomethyl-thiazol-2-yl)-amide;

2-(2-Hydroxy-3,3-dimethyl-butyrylamino)-pentanoic acid (5-formyl-thiazol-2-yl)-amide;

2-(2-Hydroxy-3,3-dimethyl-butyrylamino)-pentanoic acid [5-(1-propyl-butyl)-thiazol-2-yl]-amide;

2-Hydroxy-3,3-dimethyl-N-{1-[5-(1-propyl-butyl)-thiazol-2-ylcarbamoyl]-propyl}-butyramide;

2-Hydroxy-3,3-dimethyl-N-{1-[5-(1-propyl-butyl)-thiazol-2-ylcarbamoyl]-ethyl}-butyramide;

2-(2-Hydroxy-3,3-dimethyl-butyrylamino)-pentanoic acid (4-methyl-5-vinyl-thiazol-2-yl)-amide;

2-(2-Hydroxy-3,3-dimethyl-butyrylamino)-pentanoic acid {5-[(3-methyl-butylamino)-methyl]-thiazol-2-yl}-amide;

2-(2-Hydroxy-3,3-dimethyl-butyrylamino)-pentanoic acid {5-[(3,3-dimethyl-butylamino)-methyl]-thiazol-2-yl}-amide;

2-(2-Hydroxy-3,3-dimethyl-butyrylamino)-pentanoic acid [5-(isobutylamino-methyl)-thiazol-2-yl]-amide;

2-(2-Hydroxy-3,3-dimethyl-butyrylamino)-pentanoic acid (5-{[methyl-(3-methyl-butyl)-amino]-methyl}-thiazol-2-yl)-amide;

2-(2-Hydroxy-3,3-dimethyl-butyrylamino)-pentanoic acid [5-(1,3,3-trimethyl-butyl)-thiazol-2-yl]-amide;

2-(2-Hydroxy-3-methyl-butyrylamino)-pentanoic acid [5-(1,3,3-trimethyl-butyl)-thiazol-2-yl]-amide;

2-[2-(3,5-Difluoro-phenyl)-acetylamino]-pentanoic acid [4-methyl-5-(1-phenethylamino-ethyl)-thiazol-2-yl]-amide;

2-[2-(3,5-Difluoro-phenyl)-acetylamino]-pentanoic acid [5-(1-benzylamino-ethyl)-4-methyl-thiazol-2-yl]-amide;

2-[2-(3,5-Difluoro-phenyl)-acetylamino]-pentanoic acid (5-acetyl-thiazol-2-yl)-amide;

2-(2-Hydroxy-3-methyl-butyrylamino)-pentanoic acid [5-(5-hydroxy-1,5-dimethyl-hexyl)-thiazol-2-yl]-amide;

2-(2-Hydroxy-3-methyl-butyrylamino)-pentanoic acid [5-(1,5-dimethyl-hex-4-enyl)-thiazol-2-yl]-amide;

2-(2-Hydroxy-2-phenyl-acetylamino)-pentanoic acid [5-(1,5-dimethyl-hex-4-enyl)-thiazol-2-yl]-amide;

2-[2-(3,5-Difluoro-phenyl)-acetylamino]-pentanoic acid [5-(5-hydroxy-1,5-dimethyl-hexyl)-thiazol-2-yl]-amide;

-[2-(3,5-Difluoro-phenyl)-acetylamino]-pentanoic acid [5-(5-methoxy-1,5-dimethyl-hexyl)-thiazol-2-yl]-amide;

2-[2-(3,5-Difluoro-phenyl)-acetylamino]-pentanoic acid [5-(1,5-dimethyl-hex-4-enyl)-thiazol-2-yl]-amide;

2-[2-(3,5-Difluoro-phenyl)-2-hydroxy-acetylamino]-pentanoic acid [5-(5-hydroxy-1,5-dimethyl-hexyl)-thiazol-2-yl]-amide;

2-[2-(3,5-Difluoro-phenyl)-acetylamino]-pentanoic acid {4-methyl-5-[1-(2,2,2-trifluoro-ethylamino)-ethyl]-thiazol-2-yl}-amide;

2-[2-(3,5-Difluoro-phenyl)-acetylamino]-pentanoic acid [5-(1-dimethylamino-ethyl)-4-methyl-thiazol-2-yl]-amide;

2-[2-(3,5-Difluoro-phenyl)-acetylamino]-pentanoic acid {5-[1-(2-hydroxy-ethylamino)-ethyl]-4-methyl-thiazol-2-yl}-amide;

2-[2-(3,5-Difluoro-phenyl)-acetylamino]-pentanoic acid [5-(1-isobutylamino-ethyl)-4-methyl-thiazol-2-yl]-amide;

2-[(2-{2-[2-(3,5-Difluoro-phenyl)-acetylamino]-pentanoylamino}-thiazol-5-ylmethyl)-amino]-pentanoic acid methyl ester;

2-[2-(3,5-Difluoro-phenyl)-acetylamino]-pentanoic acid [5-(1-isopropylamino-ethyl)-thiazol-2-yl]-amide;

2-[2-(3,5-Difluoro-phenyl)-acetylamino]-pentanoic acid [5-(1-benzylamino-ethyl)-thiazol-2-yl]-amide;

2-[2-(3,5-Difluoro-phenyl)-acetylamino]-pentanoic acid {5-[1-(3,3-dimethyl-butylamino)-ethyl]-thiazol-2-yl}-amide;

2-[2-(3,5-Difluoro-phenyl)-acetylamino]-pentanoic acid {5-[1-(3-methyl-butylamino)-ethyl]-thiazol-2-yl}-amide;

2-[2-(3,5-Difluoro-phenyl)-acetylamino]-pentanoic acid (5-ethyl-4-methyl-thiazol-2-yl)-amide;

2-[2-(3,5-Difluoro-phenyl)-acetylamino]-pentanoic acid [4-methyl-5-(1-methylamino-ethyl)-thiazol-2-yl]-amide;

2-[2-(3,5-Difluoro-phenyl)-acetylamino]-pentanoic acid [4-methyl-5-(1-methylamino-ethyl)-thiazol-2-yl]-amide;

2-[(2-{2-[2-(3,5-Difluoro-phenyl)-acetylamino]-pentanoylamino}-thiazol-5-ylmethyl)-amino]-pentanoic acid;

2-[2-(3,5-Difluoro-phenyl)-acetylamino]-pentanoic acid {5-[1-(2-hydroxy-ethylamino)-ethyl]-thiazol-2-yl}-amide;

2-[2-(3,5-Difluoro-phenyl)-acetylamino]-pentanoic acid [5-(1-phenethylamino-ethyl)-thiazol-2-yl]-amide;

2-[2-(3,5-Difluoro-phenyl)-acetylamino]-pentanoic acid [5-(1-morpholin-4-yl-ethyl)-thiazol-2-yl]-amide;

2-[2-(3,5-Difluoro-phenyl)-acetylamino]-pentanoic acid (5-trifluoroacetyl-thiazol-2-yl)-amide;

2-[2-(3,5-Difluoro-phenyl)-acetylamino]-pentanoic acid [5-(1-hydroxy-3,3-dimethoxy-1-methyl-propyl)-thiazol-2-yl]-amide;

2-[2-(3,5-Difluoro-phenyl)-acetylamino]-pentanoic acid [5-(2,2,2-trifluoro-1-hydroxy-ethyl)-thiazol-2-yl]-amide;

2-[2-(3,5-Difluoro-phenyl)-acetylamino]-pentanoic acid {5-[1-(1-benzyl-pyrrolidin-3-ylamino)-ethyl]-thiazol-2-yl}-amide;

2-[2-(3,5-Difluoro-phenyl)-acetylamino]-pentanoic acid {5-[1-(2-methoxy-ethylamino)-ethyl]-thiazol-2-yl}-amide;

2-[2-(3,5-Difluoro-phenyl)-acetylamino]-pentanoic acid [5-(1-isobutylamino-ethyl)-4-methyl-thiazol-2-yl]-amide;

2-[2-(3,5-Difluoro-phenyl)-acetylamino]-pentanoic acid [5-(1-isobutylamino-ethyl)-4-methyl-thiazol-2-yl]-amide;

2-[2-(3,5-Difluoro-phenyl)-acetylamino]-pentanoic acid [5-(1-propylamino-ethyl)-thiazol-2-yl]-amide; and

2-[2-(3,5-Difluoro-phenyl)-acetylamino]-pentanoic acid {5-[1-(3,3-dimethyl-butylamino)-2,2,2-trifluoro-ethyl]-thiazol-2-yl}-amide.

Other compounds of Formula I encompassed by the present invention are:

2-Benzenesulfonylamino-pentanoic acid [5-(1-ethyl-propyl)-thiazol-2-yl]-amide

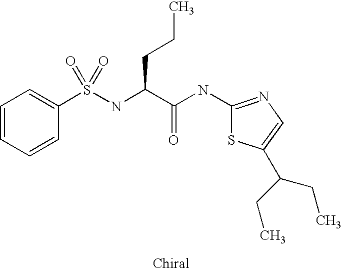

and

2-(4-Chloro-benzenesulfonylamino)-pentanoic acid [5-(5-hydroxy-1,5-dimethyl-hexyl)-thiazol-2-yl]-amide

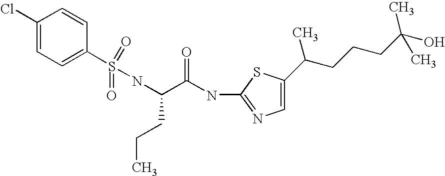

and pharmaceutically acceptable salts thereof.

Of the above compounds, more preferred compounds of Formula I are:

2-[2-(3,5-Difluoro-phenyl)-acetylamino]-pentanoic acid [5-(1-propyl-butyl)-thiazol-2-yl]-amide;

2-[2-(3,5-Difluoro-phenyl)-acetylamino]-pentanoic acid [5-(1-ethyl-1-hydroxy-propyl)-thiazol-2-yl]-amide;

2-(2-Hydroxy-3,3-dimethyl-butyrylamino)-pentanoic acid [5-(1-ethyl-1-hydroxy-propyl)-thiazol-2-yl]-amide;

2-[2-(3,5-Difluoro-phenyl)-acetylamino]-pentanoic acid [5-(1-butylamino-ethyl)-4-methyl-thiazol-2-yl]-amide;

2-[2-(3,5-Difluoro-phenyl)-acetylamino]-pentanoic acid [4-methyl-5-(1-propylamino-ethyl)-thiazol-2-yl]-amide;

2-[2-(3,5-Difluoro-phenyl)-acetylamino]-pentanoic acid [5-(3,3-dimethyl-cyclohexyl)-thiazol-2-yl]-amide;

2-[2-(3,5-Difluoro-phenyl)-acetylamino]-pentanoic acid (5-ethyl-thiazol-2-yl)-amide;

2-[2-(3,5-Difluoro-phenyl)-acetylamino]-pentanoic acid [5-(1-benzyl-4-hydroxy-piperidin-4-yl)-thiazol-2-yl]-amide;

2-[2-(3,5-Difluoro-phenyl)-acetylamino]-pentanoic acid [5-(1-hydroxy-ethyl)-4-methyl-thiazol-2-yl]-amide;

2-[2-(3,5-Difluoro-phenyl)-acetylamino]-pentanoic acid [5-(1-hydroxy-ethyl)-4-methyl-thiazol-2-yl]-amide;

2-(2-Hydroxy-2-phenyl-acetylamino)-pentanoic acid [5-(1-ethyl-1-hydroxy-propyl)-thiazol-2-yl]-amide;

2-[2-(3,5-Difluoro-phenyl)-acetylamino]-pentanoic acid {5-[1-(2-methoxy-ethylamino)-ethyl]-4-methyl-thiazol-2-yl}-amide;

2-[2-(3,5-Difluoro-phenyl)-acetylamino]-pentanoic acid [4-methyl-5-(1-pyrrolidin-1-yl-ethyl)-thiazol-2-yl]-amide;

2-(2-Hydroxy-3,3-dimethyl-butyrylamino)-pentanoic acid [5-(1-ethyl-propyl)-thiazol-2-yl]-amide;

2-[2-(3-Phenoxy-phenyl)-acetylamino]-pentanoic acid [5-(1-ethyl-propyl)-thiazol-2-yl]-amide;

2-(2-Hydroxy-3-methyl-butyrylamino)-pentanoic acid [5-(1-ethyl-propyl)-thiazol-2-yl]-amide;

2-(2-Hydroxy-3-methyl-butyrylamino)-pentanoic acid [5-(1-ethyl-propyl)-thiazol-2-yl]-amide;

2-[2-(3,5-Difluoro-phenyl)-2-hydroxy-acetylamino]-pentanoic acid [5-(1-ethyl-propyl)-thiazol-2-yl]-amide;

2-[2-(5-Bromo-pyridin-3-yl)-acetylamino]-pentanoic acid [5-(1-ethyl-propyl)-thiazol-2-yl]-amide;

2-[2-(3,5-Difluoro-phenyl)-acetylamino]-pentanoic acid [4-methyl-5-(1-methylamino-ethyl)-thiazol-2-yl]-amide;

2-[2-(3,5-Difluoro-phenyl)-acetylamino]-pentanoic acid [5-(1-ethylamino-ethyl)-4-methyl-thiazol-2-yl]-amide;

2-[2-(3,5-Difluoro-phenyl)-acetylamino]-pentanoic acid [4-methyl-5-(1-morpholin-4-yl-ethyl)-thiazol-2-yl]-amide;

2-[2-(3,5-Difluoro-phenyl)-acetylamino]-N-[5-(1-ethyl-propyl)-thiazol-2-yl]-propionamide;

N-{1-[5-(1-Ethyl-propyl)-thiazol-2-ylcarbamoyl]-ethyl}-2-hydroxy-3,3-dimethyl-butyramide;

2-(2-Hydroxy-2-phenyl-acetylamino)-pentanoic acid [5-(1-ethyl-propyl)-thiazol-2-yl]-amide;

2-[2-(3,5-Difluoro-phenyl)-acetylamino]-pentanoic acid [5-(1-isobutylamino-ethyl)-4-methyl-thiazol-2-yl]-amide;

2-[2-(3,5-Difluoro-phenyl)-acetylamino]-pentanoic acid {4-methyl-5-[1-(3-methyl-butylamino)-ethyl]-thiazol-2-yl}-amide;

2-[2-(3,5-Difluoro-phenyl)-acetylamino]-pentanoic acid (5-morpholin-4-ylmethyl-thiazol-2-yl)-amide;

2-[2-(3,5-Difluoro-phenyl)-2-hydroxy-acetylamino]-pentanoic acid [5-(5-methoxy-1,5-dimethyl-hexyl)-thiazol-2-yl]-amide;

N-[5-(1-Ethyl-propyl)-thiazol-2-yl]-2-(2-hydroxy-2-phenyl-acetylamino)-propionamide;

2-(2-Hydroxy-3,3-dimethyl-butyrylamino)-pentanoic acid [5-(1-propyl-butyl)-thiazol-2-yl]-amide;

2-Hydroxy-3,3-dimethyl-N-{1-[5-(1-propyl-butyl)-thiazol-2-ylcarbamoyl]-propyl}-butyramide;

2-Hydroxy-3,3-dimethyl-N-{1-[5-(1-propyl-butyl)-thiazol-2-ylcarbamoyl]-ethyl}-butyramide;

2-(2-Hydroxy-3,3-dimethyl-butyrylamino)-pentanoic acid {5-[(3,3-dimethyl-butylamino)-methyl]-thiazol-2-yl}-amide;

2-(2-Hydroxy-3,3-dimethyl-butyrylamino)-pentanoic acid [5-(isobutylamino-methyl)-thiazol-2-yl]-amide;

2-(2-Hydroxy-3,3-dimethyl-butyrylamino)-pentanoic acid (5-{[methyl-(3-methyl-butyl)-amino]-methyl}-thiazol-2-yl)-amide;

2-(2-Hydroxy-3,3-dimethyl-butyrylamino)-pentanoic acid [5-(1,3,3-trimethyl-butyl)-thiazol-2-yl]-amide;

2-(2-Hydroxy-3-methyl-butyrylamino)-pentanoic acid [5-(1,3,3-trimethyl-butyl)-thiazol-2-yl]-amide;

2-[2-(3,5-Difluoro-phenyl)-acetylamino]-pentanoic acid [4-methyl-5-(1-phenethylamino-ethyl)-thiazol-2-yl]-amide;

2-[2-(3,5-Difluoro-phenyl)-acetylamino]-pentanoic acid [5-(1-benzylamino-ethyl)-4-methyl-thiazol-2-yl]-amide;

2-(2-Hydroxy-3-methyl-butyrylamino)-pentanoic acid [5-(5-hydroxy-1,5-dimethyl-hexyl)-thiazol-2-yl]-amide;

2-[2-(3,5-Difluoro-phenyl)-acetylamino]-pentanoic acid [5-(5-hydroxy-1,5-dimethyl-hexyl)-thiazol-2-yl]-amide;

2-[2-(3,5-Difluoro-phenyl)-acetylamino]-pentanoic acid {4-methyl-5-[1-(2,2,2-trifluoro-ethylamino)-ethyl]-thiazol-2-yl}-amide;

2-[(2-{2-[2-(3,5-Difluoro-phenyl)-acetylamino]-pentanoylamino}-thiazol-5-ylmethyl)-amino]-pentanoic acid methyl ester;

2-(2-Hydroxy-3,3-dimethyl-butyrylamino)-pentanoic acid (5-isopropyl-thiazol-2-yl)-amide;

2-(2-Hydroxy-2-phenyl-acetylamino)-pentanoic acid (5-isopropyl-thiazol-2-yl)-amide;

2-[2-(3,5-Difluoro-phenyl)-acetylamino]-hexanoic acid (5-isopropyl-thiazol-2-yl)-amide;

2-[2-(5-Bromo-pyridin-3-yl)-acetylamino]-N-[5-(5-hydroxy-1,5-dimethyl-hexyl)-thiazol-2-yl]-propionamide;

2-[2-(3,5-Difluoro-phenyl)-acetylamino]-pentanoic acid [5-(1,3,3-trimethyl-butyl)-thiazol-2-yl]-amide;

2-(2-Hydroxy-3,3-dimethyl-butyrylamino)-pentanoic acid [5-(5-methoxy-1,5-dimethyl-hexyl)-thiazol-2-yl]-amide;

2-(2-Hydroxy-3-methyl-butyrylamino)-pentanoic acid [5-(5-methoxy-1,5-dimethyl-hexyl)-thiazol-2-yl]-amide;

2-(2-Oxo-2-thiophen-2-yl-acetylamino)-pentanoic acid [5-(5-hydroxy-1,5-dimethyl-hexyl)-thiazol-2-yl]-amide;

2-(3,3-Dimethyl-2-oxo-butyrylamino)-pentanoic acid (5-isopropyl-thiazol-2-yl)-amide;

2-(2-Hydroxy-2-phenyl-acetylamino)-pentanoic acid [5-(5-hydroxy-1,5-dimethyl-hexyl)-thiazol-2-yl]-amide;

2-[2-(3,5-Difluoro-phenyl)-acetylamino]-N-[5-(5-methoxy-1,5-dimethyl-hexyl)-thiazol-2-yl]-butyramide;

2-(2-Hydroxy-2-phenyl-acetylamino)-pentanoic acid [5-(5-methoxy-1,5-dimethyl-hexyl)-thiazol-2-yl]-amide;

2-(2-Hydroxy-2-phenyl-acetylamino)-N-(5-isopropyl-thiazol-2-yl)-butyramide;

2-(2-Hydroxy-2-phenyl-acetylamino)-N-(5-isopropyl-thiazol-2-yl)-propionamide;

2-[2-(5-Bromo-pyridin-3-yl)-acetylamino]-N-(5-isopropyl-thiazol-2-yl)-butyramide;

2-[2-(3,5-Difluoro-phenyl)-acetylamino]-pentanoic acid [5-(1-butylamino-ethyl)-thiazol-2-yl]-amide;

2-[2-(3,5-Difluoro-phenyl)-acetylamino]-pentanoic acid [5-(1-ethyl-propyl)-thiazol-2-yl]-amide;

2-(2-Hydroxy-3,3-dimethyl-butyrylamino)-pentanoic acid [5-(1,5-dimethyl-hex-4-enyl)-thiazol-2-yl]-amide;

2-(2-Hydroxy-3,3-dimethyl-butyrylamino)-pentanoic acid [5-(5-methoxy-1,5-dimethyl-hexyl)-thiazol-2-yl]-amide;

2-(2-Hydroxy-3,3-dimethyl-butyrylamino)-pentanoic acid [5-(5-methoxy-1,5-dimethyl-hexyl)-thiazol-2-yl]-amide;

2-(2-Hydroxy-3,3-dimethyl-butyrylamino)-pentanoic acid [5-(5-hydroxy-1,5-dimethyl-hexyl)-thiazol-2-yl]-amide;

2-(2-Hydroxy-3,3-dimethyl-butyrylamino)-pentanoic acid [5-(5-hydroxy-1,5-dimethyl-hexyl)-thiazol-2-yl]-amide;

2-(2-Hydroxy-3-methyl-butyrylamino)-pentanoic acid [5-(5-methoxy-1,5-dimethyl-hexyl)-thiazol-2-yl]-amide; and

2-(2-Hydroxy-3-methyl-butyrylamino)-pentanoic acid [5-(5-methoxy-1,5-dimethyl-hexyl)-thiazol-2-yl]-amide;

and pharmaceutically acceptable salts thereof.

Other preferred compounds of Formula I are compounds having the following structures, and all pharmaceutically acceptable salts thereof, complexes thereof, and derivatives thereof which convert into a pharmaceutically active compound upon administration:

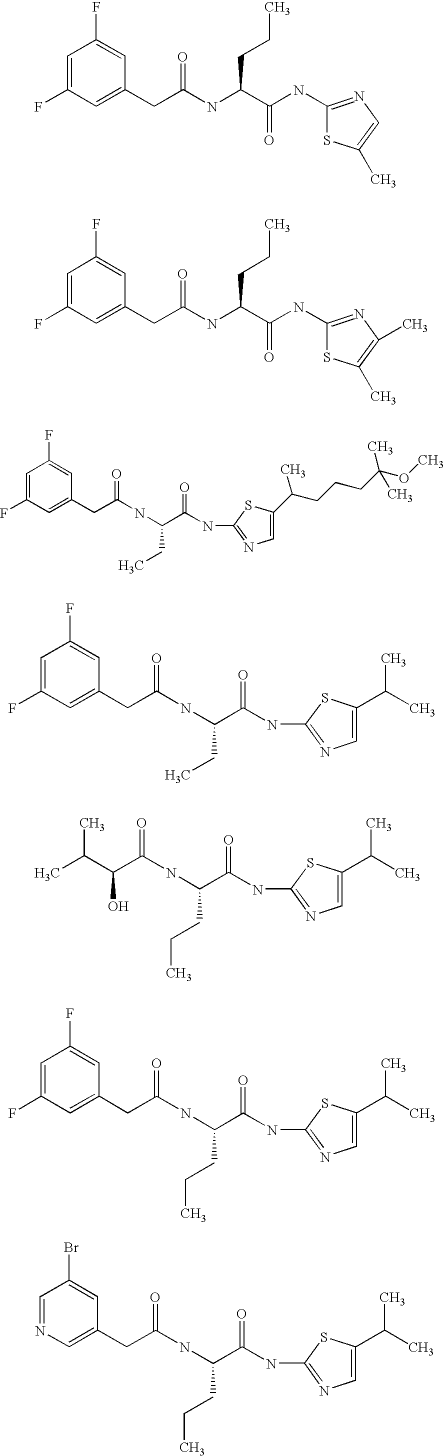

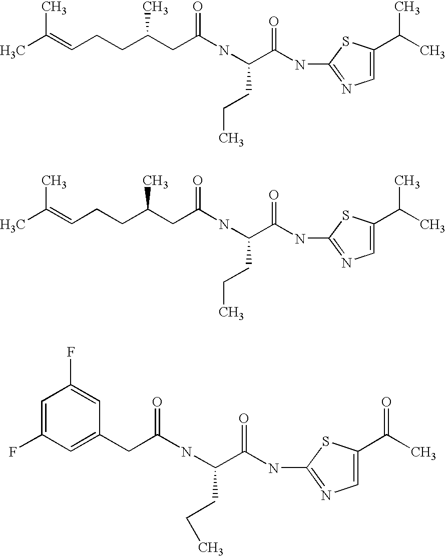

The present invention also provides compounds of Formula

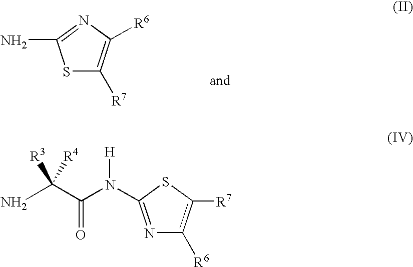

wherein

R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);

R⁴ is H, D, F, or C₁-C₄ alkyl;

or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;

R⁶ is selected from —H, —C₁-C₂₀ alkyl, —Cl, —F, —Br, —I, —CN, —CF₃, —C(═O)R¹¹, —C(═O)OR¹², —S(O)ₙNR⁹R¹⁰, —S(O)ₙR¹¹, —C(═NR⁹)R¹⁵, -(C₃-C₁₂) cycloalkyl, -(C₄-C₁₂) cycloalkenyl, and —C₆-C₁₀ aryl, wherein said alkyl, alkylene, cycloalkyl, cycloalkenyl, and aryl of R⁶ are each optionally substituted with from one to three substituents R^{1b};

R⁷ is selected from H, —Cl, —F, —Br, —I, —CN, —NO₂, —NR¹⁴R¹⁵, —CF₃, —C(═O)NR¹⁴R¹⁵, —C(═O)R¹³, —S(O)ₙR¹³, —C(═O)OR¹³, —C(═NR⁹)R¹⁵, —S(O)ₙNR¹⁴R¹⁵, —C₁-C₂₀ alkyl, —C₁-C₂₀ alkoxy, -(C_{zero}-C₄ alkylene)-(C₃-C₁₂ cycloalkyl), -(C_{zero}-C₄ alkylene)-((C₄-C₁₂)cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-((3-12 membered) heterocycloalkyl), -(C_{zero}-C₄ alkylene)-((7-20 membered) heterobi- or heterotricycloalkyl), -(C_{zero}-C₄ alkylene)-((C₆-C₁₄)aryl), and -(C_{zero}-C₄ alkylene)-((5-14 membered) heteroaryl); wherein R⁷ is optionally substituted with from one to three substituents independently selected from R^{1a}, -(CH₂)₁₋₁₀NR⁹R¹⁰, —C₃-C₁₂ cycloalkyl, -((4-12 membered) heterocycloalkyl), -(C₆-C₁₄) aryl, -((5-15 membered) heteroaryl), -(4-12 membered) heterocycloalkoxy), -(C₆-C₁₂) aryloxy and -((5-12 membered) heteroaryloxy); said cycloalkyl, cycloalkenyl, bi- or tricycloalkyl, bi- or tricycloalkenyl, heterocycloalkyl, aryl, and heteroaryl of R⁷ are each optionally and independently substituted with from one to six F; said alkyl, alkoxy, and alkylene of R⁷ each optionally contains from one to five double or triple bonds; and each hydrogen atom of said alkyl, alkoxy, and alkylene of R⁷ is independently optionally replaced with a fluorine;

or R⁶ and R⁷ may together optionally form a -(C₆-C₁₀) aryl ring, -(C₆-C₈) cycloalkyl or cycloalkenyl ring, a five to eight membered heterocycloalkyl or heterocycloalkenyl ring, a -(C₁₀-C₁₄) membered bicycloalkyl or bicycloalkenyl ring, or a ten to fourteen membered heterobicycloalkyl or heterobicycloalkenyl ring fused to the thiazole ring of Formula I, wherein from one to three members of said heterocycloalkyl and heterocycloalkenyl rings, and from one to five members of said heterobicycloalkyl and heterobicycloalkenyl rings are selected independently from N—R⁹, O and S(O)_{zero-2}, and wherein said aryl, cycloalkyl, cycloalkenyl, heterocycloalkyl, heterocycloalkenyl, bicycloalkyl, bicycloalkenyl, heterobicycloalkyl, and heterobicycloalkenyl rings optionally are substituted with from one to three R^{1b};

R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇-C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;

or NR⁹R¹⁰ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;

R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};

R¹³ is selected from H, —C₁-C₆ alkyl optionally containing from one to three double or triple bonds and wherein each hydrogen is independently optionally replaced with a fluorine, -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), and -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-((3-12 membered) heterocycloalkyl), -(C_{zero}-C₄ alkylene)-((7-20 membered) heterobi- or heterotricycloalkyl), and -(C_{zero}-C₄ alkylene)-((5-14 membered) heteroaryl), and R¹³ is optionally substituted with from one to three substituents R^{1b};

R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;

or NR¹⁴R¹⁵ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I;

R^{1a} is in each instance independently selected from —OH, —C₁-C₆ alkyl independently optionally containing from one to three double or triple bonds, —C₁-C₆ alkoxy independently optionally containing from one to three double or triple bonds, —Cl, —F, —Br, —I, —CN, —NO₂, —NR⁹R¹⁰, —C(═O)NR⁹R¹⁰, —S(O)ₙNR⁹R¹⁰, —C(═O)R¹¹, —S(O)ₙR¹¹, —C(═O)OR¹², —C₃-C₈ cycloalkyl, —C₄-C₈ cycloalkenyl, -(C₅-C₁₁)bi- or tricycloalkyl, -(C₇-C₁₁)bi- or tricycloalkenyl, -(3-8 membered) heterocycloalkyl, -(C₆-C₁₄)aryl, -(5-14 membered) heteroaryl, -(C₆-C₁₄) aryloxy, and -(5-14 membered) heteroaryloxy, wherein said alkyl, alkoxy, cycloalkyl, cycloalkenyl, bi- or tricycloalkyl, bi- or tricycloalkenyl, heterocycloalkyl, aryl, heteroaryl, aryloxy, and heteroaryloxy are each independently optionally substituted with from one to three substituents R^{1b};

R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, and —C₁-C₆ alkyl independently optionally containing from one to three double or triple bonds and independently substituted with from one to six atoms independently selected from F, Cl, Br, and I; and

n is in each instance an integer independently selected from zero, 1, 2, and 3.

Compounds of Formula II and Formula IV are useful as intermediates for synthesis of compounds of Formula I.

This invention also provides compounds of Formula

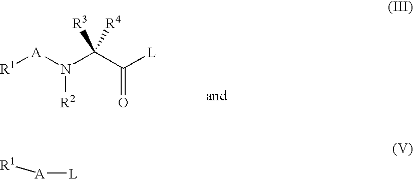

wherein:

A is selected from —C(═O)C(═O)—, —C(═O)NR⁹—, —C(═O)Z-, —C(═S)Z-, —C(═NR⁵)Z-, and —S(O)₂—;

wherein Z is —CH₂—, —CH(OH)—, —CH(OC(═O)R¹¹)—, —CH(NH₂)—, —CH(CH₂(OH))—, —CH(CH(C₁-C₄ alkyl)(OH))—, or —CH(C(C₁-C₄ alkyl)(C₁-C₄ alkyl)(OH))—, for example —CH(C(CH₃)(CH₃)(OH))— or —CH(C(CH₃)(CH₂CH₃)(OH))—;

R¹ is selected from C₁-C₂₀ alkyl and —C₁-C₂₀ alkoxy, C₃-C₈ cycloalkyl, (C₄-C₈)cycloalkenyl, (C₅-C₁₁)bi- or tricycloalkyl, (C₇-C₁₁)bi- or tricycloalkenyl, (3-8 membered) heterocycloalkyl, (C₆-C₁₄)aryl, or (5-14 membered) heteroaryl, wherein said alkyl and alkoxy each optionally contains from one to five double or triple bonds, and wherein each hydrogen atom of said alkyl and alkoxy is optionally replaced with a fluorine;

wherein when R¹ is alkyl or alkoxy, R¹ is optionally substituted with from one to three substituents R^{1a}, and wherein when R¹ is cycloalkyl, cycloalkenyl, bi- or tricycloalkyl, bi- or tricycloalkenyl, heterocycloalkyl, aryl, or heteroaryl, then R¹ is optionally substituted with from one to three substituents R^{1b};

R^{1a} is in each instance independently selected from —OH, —C₁-C₆ alkyl independently optionally containing from one to three double or triple bonds, —C₁-C₆ alkoxy independently optionally containing from one to three double or triple bonds, —Cl, —F, —Br, —I, —CN, —NO₂, —NR⁹R¹⁰, —C(═O)NR⁹R¹⁰, —S(O)ₙNR⁹R¹⁰, —C(═O)R¹¹, —S(O)ₙR¹¹, —C(═O)OR¹², —C₃-C₈ cycloalkyl, —C₄-C₈ cycloalkenyl, -(C₅-C₁₁)bi- or tricycloalkyl, -(C₇-C₁₁)bi- or tricycloalkenyl, -(3-8 membered) heterocycloalkyl, -(C₆-C₁₄)aryl, -(5-14 membered) heteroaryl, -(C₆-C₁₄) aryloxy, and -(5-14 membered) heteroaryloxy, wherein said alkyl, alkoxy, cycloalkyl, cycloalkenyl, bi- or tricycloalkyl, bi- or tricycloalkenyl, heterocycloalkyl, aryl, heteroaryl, aryloxy, and heteroaryloxy are each independently optionally substituted with from one to three substituents R^{1b};

R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, and —C₁-C₆ alkyl independently optionally containing from one to three double or triple bonds and independently substituted with from one to six atoms independently selected from F, Cl, Br, and I;

R² is selected from —H, —C₁-C₄ alkyl optionally containing one or two double or triple bonds, —C(═O)(C₁-C₄ alkyl), —C₆-C₁₀ aryl, —SO₂—(C₆-C₁₀ aryl), and —SO₂—CH₂—(C₆-C₁₀ aryl), and R² is optionally substituted with from one to three substituents R^{1b};

R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);

R⁴ is H, D, F, or C₁-C₄ alkyl;

or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;

R⁵ is selected from —H, —C₁-C₆ alkyl optionally substituted with from one to three R^{1a}, and —C₆-C₁₀ aryl optionally substituted with from one to three R^{1a};

or R⁵ and R¹ may together optionally form a five to fourteen membered heteroaryl ring or a five to eight membered heterocycloalkyl ring, wherein said heteroaryl ring optionally contains one or two further heteroatoms independently selected from N, O, and S, and said heterocycloalkyl ring optionally contains one or two further heteroatoms independently selected from N—R⁹, O, and S(O)_{zero-2}, and wherein said heterocycloalkyl ring optionally contains from one to three double bonds, and wherein said heteroaryl or heterocycloalkyl ring is optionally substituted from one to three substituents R^{1b};

R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇-C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;

or NR⁹R¹⁰ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;

R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};

R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;

or NR¹⁴R¹⁵ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I;

n is in each instance an integer independently selected from zero, 1, 2, and 3; and

L is hydroxy or a suitable leaving group; or

A-L is an alkyl ester or an aryl ester.

Compounds of Formula III and compounds of Formula V are useful as intermediates for synthesizing compounds of Formula I.

In one embodiment of the invention, compounds of Formula III are provided wherein L is hydroxy or a halogen atom. In another embodiment, compounds of Formula III are provided wherein L is hydroxy or —Cl, —Br, or —I. In another embodiment, compounds of Formula III are provided wherein A-L is an alkyl ester or an aryl ester.

In another embodiment of the invention, compounds of Formula V are provided wherein L is hydroxy or a halogen atom. In another embodiment, compounds of Formula V are provided wherein L is hydroxy or —Cl, —Br, or —I. In another embodiment, compounds of Formula V are provided wherein A-L is an alkyl ester or an aryl ester.

The present invention also provides compounds of Formula

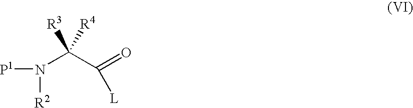

wherein:

R² is selected from —H, —C₁-C₄ alkyl optionally containing one or two double or triple bonds, —C(═O)(C₁-C₄ alkyl), —C₆-C₁₀ aryl, —SO₂—(C₆-C₁₀ aryl), and —SO₂—CH₂—(C₆-C₁₀ aryl), and R² is optionally substituted with from one to three substituents R^{1b};

R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);

R⁴ is H, D, F, or C₁-C₄ alkyl;

or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;

R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, and —C₁-C₆ alkyl independently optionally containing from one to three double or triple bonds and independently substituted with from one to six atoms independently selected from F, Cl, Br, and I;

R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;

or NR⁹R¹⁰ can independently optionally form a heterocycloalkyl moiety of from four to seven ring members, said heterocycloalkyl moiety independently optionally comprising one or two furthers independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;

R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};

R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;

or NR¹⁴R¹⁵ can independently optionally form a heterocycloalkyl moiety of from four to seven ring members, said heterocycloalkyl moiety independently optionally comprising one or two furthers independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I;

n is in each instance an integer independently selected from zero, 1, 2, and 3;

L is hydroxy or a suitable leaving group; and

P¹ is an amino protecting group.

Examples of amino protecting groups include, but are not limited to, N-Boc, benzyl, p-methoxy-benzyl, trimethylsilyl, and t-butyldimethylsilyl.

Compounds of Formula VI are useful as intermediates for synthesizing compounds of Formula I.

The present invention also provides methods of synthesizing compounds of Formula

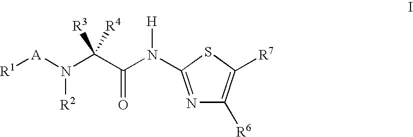

and pharmaceutically acceptable salts thereof,

wherein:

A is selected from —C(═O)C(═O)—, —C(═O)NR⁹—, —C(═O)Z-, —C(═S)Z-, —C(═NR⁵)Z-, and —S(O)₂—;

wherein Z is —CH₂—, —CH(OH)—, —CH(OC(═O)R¹¹)—, —CH(NH₂)—, —CH(CH₂(OH))—, —CH(CH(C₁-C₄ alkyl)(OH))—, or —CH(C(C₁-C₄ alkyl)(C₁-C₄ alkyl)(OH))—, for example —CH(C(CH₃)(CH₃)(OH))— or —CH(C(CH₃)(CH₂CH₃)(OH))—;

R¹ is selected from C₁-C₂₀ alkyl and —C₁-C₂₀ alkoxy, C₃-C₈ cycloalkyl, (C₄-C₈)cycloalkenyl, (C₅-C₁₁)bi- or tricycloalkyl, (C₇-C₁₁)bi- or tricycloalkenyl, (3-8 membered) heterocycloalkyl, (C₆-C₁₄)aryl, or (5-14 membered) heteroaryl, wherein said alkyl and alkoxy each optionally contains from one to five double or triple bonds, and wherein each hydrogen atom of said alkyl and alkoxy is optionally replaced with a fluorine;

wherein when R¹ is alkyl or alkoxy, R¹ is optionally substituted with from one to three substituents R^{1a}, and wherein when R¹ is cycloalkyl, cycloalkenyl, bi- or tricycloalkyl, bi- or tricycloalkenyl, heterocycloalkyl, aryl, or heteroaryl, then R¹ is optionally substituted with from one to three substituents R^{1b};

R^{1a} is in each instance independently selected from —OH, —C₁-C₆ alkyl independently optionally containing from one to three double or triple bonds, —C₁-C₆ alkoxy independently optionally containing from one to three double or triple bonds, —Cl, —F, —Br, —I, —CN, —NO₂, —NR⁹R¹⁰, —C(═O)NR⁹R¹⁰, —S(O)ₙNR⁹R¹⁰, —C(═O)R¹¹, —S(O)ₙR¹¹, —C(═O)OR¹², —C₃-C₈ cycloalkyl, —C₄-C₈ cycloalkenyl, -(C₅-C₁₁)bi- or tricycloalkyl, -(C₇-C₁₁)bi- or tricycloalkenyl, -(3-8 membered) heterocycloalkyl, -(C₆-C₁₄)aryl, -(5-14 membered) heteroaryl, -(C₆-C₁₄) aryloxy, and -(5-14 membered) heteroaryloxy, wherein said alkyl, alkoxy, cycloalkyl, cycloalkenyl, bi- or tricycloalkyl, bi- or tricycloalkenyl, heterocycloalkyl, aryl, heteroaryl, aryloxy, and heteroaryloxy are each independently optionally substituted with from one to three substituents R^{1b};

R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, and —C₁-C₆ alkyl independently optionally containing from one to three double or triple bonds and independently substituted with from one to six atoms independently selected from F, Cl, Br, and I;

R² is selected from —H, —C₁-C₄ alkyl optionally containing one or two double or triple bonds, —C(═O)(C₁-C₄ alkyl), —C₆-C₁₀ aryl, —SO₂—(C₆-C₁₀ aryl), and —SO₂—CH₂—(C₆-C₁₀ aryl), and R² is optionally substituted with from one to three substituents R^{1b};

R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);

R⁴ is H, D, F, or C₁-C₄ alkyl;

or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;

R⁵ is selected from —H, —C₁-C₆ alkyl optionally substituted with from one to three R^{1a}, and —C₆-C₁₀ aryl optionally substituted with from one to three R^{1a};

or R⁵ and R¹ may together optionally form a five to fourteen membered heteroaryl ring or a five to eight membered heterocycloalkyl ring, wherein said heteroaryl ring optionally contains one or two further heteroatoms independently selected from N, O, and S, and said heterocycloalkyl ring optionally contains one or two further heteroatoms independently selected from N—R⁹, O, and S(O)_{zero-2}, and wherein said heterocycloalkyl ring optionally contains from one to three double bonds, and wherein said heteroaryl or heterocycloalkyl ring is optionally substituted from one to three substituents R^{1b};

R⁶ is selected from —H, —C₁-C₂₀ alkyl, —Cl, —F, —Br, —I, —CN, —CF₃, —C(═O)R¹¹, —C(═O)OR¹², —S(O)ₙNR⁹R¹⁰, —S(O)ₙR¹¹, —C(═NR⁹)R¹⁵, -(C₃-C₁₂) cycloalkyl, -(C₄-C₁₂) cycloalkenyl, and —C₆-C₁₀ aryl, wherein said alkyl, alkylene, cycloalkyl, cycloalkenyl, and aryl of R⁶ are each optionally substituted with from one to three substituents R^{1b};

R⁷ is selected from H, —Cl, —F, —Br, —I, —CN, —NO₂, —NR¹⁴R¹⁵, —CF₃, —C(═O)NR¹⁴R¹⁵, —C(═O)R¹³, —S(O)ₙR¹³, —C(═O)OR¹³, —C(═NR⁹)R¹⁵, —S(O)ₙNR¹⁴R¹⁵, —C₁-C₂₀ alkyl, —C₁-C₂₀ alkoxy, -(C_{zero}-C₄ alkylene)-(C₃-C₁₂ cycloalkyl), -(C_{zero}-C₄ alkylene)-((C₄-C₁₂)cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-((3-12 membered) heterocycloalkyl), -(C_{zero}-C₄ alkylene)-((7-20 membered) heterobi- or heterotricycloalkyl), -(C_{zero}-C₄ alkylene)-((C₆-C₁₄)aryl), and -(C_{zero}-C₄ alkylene)-((5-14 membered) heteroaryl); wherein R⁷ is optionally substituted with from one to three substituents independently selected from R^{1a}, -(CH₂)₁₋₁₀NR⁹R¹⁰, —C₃-C₁₂ cycloalkyl, -((4-12 membered) heterocycloalkyl), -(C₆-C₁₄) aryl, -((5-15 membered) heteroaryl), -(4-12 membered) heterocycloalkoxy), -(C₆-C₁₂) aryloxy and -((5-12 membered) heteroaryloxy); said cycloalkyl, cycloalkenyl, bi- or tricycloalkyl, bi- or tricycloalkenyl, heterocycloalkyl, aryl, and heteroaryl of R⁷ are each optionally and independently substituted with from one to six F; said alkyl, alkoxy, and alkylene of R⁷ each optionally contains from one to five double or triple bonds; and each hydrogen atom of said alkyl, alkoxy, and alkylene of R⁷ is independently optionally replaced with a fluorine;

or R⁶ and R⁷ may together optionally form a -(C₆-C₁₀) aryl ring, -(C₆-C₈) cycloalkyl or cycloalkenyl ring, a five to eight membered heterocycloalkyl or heterocycloalkenyl ring, a -(C₁₀-C₁₄) membered bicycloalkyl or bicycloalkenyl ring, or a ten to fourteen membered heterobicycloalkyl or heterobicycloalkenyl ring fused to the thiazole ring of Formula I, wherein from one to three members of said heterocycloalkyl and heterocycloalkenyl rings, and from one to five members of said heterobicycloalkyl and heterobicycloalkenyl rings are selected independently from N—R⁹, O and S(O)_{zero-2}, and wherein said aryl, cycloalkyl, cycloalkenyl, heterocycloalkyl, heterocycloalkenyl, bicycloalkyl, bicycloalkenyl, heterobicycloalkyl, and heterobicycloalkenyl rings optionally are substituted with from one to three R^{1b};

R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇-C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;

or NR⁹R¹⁰ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;

R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};

R¹³ is selected from H, —C₁-C₆ alkyl optionally containing from one to three double or triple bonds and wherein each hydrogen is independently optionally replaced with a fluorine, -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), and -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-((3-12 membered) heterocycloalkyl), -(C_{zero}-C₄ alkylene)-((7-20 membered) heterobi- or heterotricycloalkyl), and -(C_{zero}-C₄ alkylene)-((5-14 membered) heteroaryl), and R¹³ is optionally substituted with from one to three substituents R^{1b};

R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;

or NR¹⁴R¹⁵ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I; and

n is in each instance an integer independently selected from zero, 1, 2, and 3.

In one embodiment, a compound of Formula

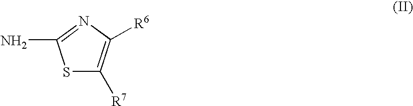

wherein R⁶ and R⁷ are as defined above,

is reacted with a compound of Formula

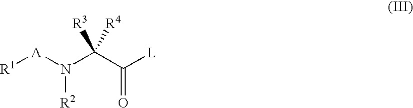

wherein R¹, R², R³, R⁴, and A are as defined above, and is hydroxy or a suitable leaving group.

In another embodiment, a method for synthesis of a compound of Formula I is provided wherein a compound of Formula IV

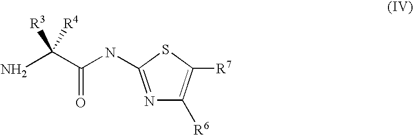

wherein R³, R⁴, R⁶ and R⁷ are as defined above;

is reacted with a compound of Formula

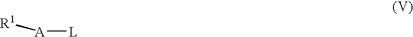

wherein R¹ and A are as defined above, and L is hydroxy or a suitable leaving group;

or R¹ is as defined above, and A-L is an alkyl ester or an aryl ester.

The invention further provides a method for synthesizing a compound of Formula I as described in the preceding paragraph, wherein the compound of Formula IV is obtained by reacting a compound of Formula

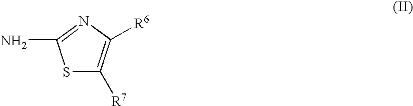

wherein R⁶ and R⁷ are as defined above;

with a compound of Formula

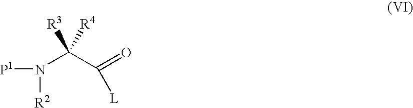

wherein R², R³, and R⁴ are as defined above; L is hydroxy or a suitable leaving group; and P¹ is an amino protecting group.

The present invention also provides a pharmaceutical composition for treating in a mammal, including in a human, a disease or condition associated with Aβ-peptide production, which pharmaceutical composition comprises a compound of Formula I in an amount effective in inhibiting Aβ-production and a pharmaceutically acceptable carrier.

The present invention also provides a pharmaceutical composition for treating in a mammal, including in a human, a disease or condition associated with Aβ-peptide production, which pharmaceutical composition comprises a compound of Formula I in an amount effective in inhibiting said disease or condition and a pharmaceutically acceptable carrier.

The present invention also provides a pharmaceutical composition for treating in a mammal, including in a human, a disease or condition selected from Alzheimer's disease, hereditary cerebral hemorrhage with amyloidosis, cerebral amyloid angiopathy, a prion-mediated disease, inclusion body myositis, stroke, and Down's Syndrome, which pharmaceutical composition comprises a compound of Formula I in an amount effective in inhibiting Aβ-production and a pharmaceutically acceptable carrier.

The present invention also provides a pharmaceutical composition for treating in a mammal, including in a human, a disease or condition selected from Alzheimer's disease, hereditary cerebral hemorrhage with amyloidosis, cerebral amyloid angiopathy, a prion-mediated disease, inclusion body myositis, stroke, and Down's Syndrome, which pharmaceutical composition comprises a compound of Formula I in an amount effective in inhibiting said disease or condition and a pharmaceutically acceptable carrier.

The present invention also provides a method for treating in a mammal, including in a human, a disease or condition associated with Aβ-peptide production, which method comprises administering to said mammal an amount of a compound of Formula I effective in inhibiting Aβ-production.

The present invention also provides a method for treating in a mammal, including in a human, a disease or condition associated with Aβ-peptide production, which method comprises administering to said mammal an amount of a compound of Formula I effective in treating said disease or condition.

The present invention also provides a method for treating in a mammal, including in a human, a disease or condition selected from Alzheimer's disease, hereditary cerebral hemorrhage with amyloidosis, cerebral amyloid angiopathy, a prion-mediated disease, inclusion body myositis, stroke, and Down's Syndrome, which method comprises administering to said mammal an amount of a compound of Formula I effective in inhibiting Aβ-production.

The present invention also provides a method for treating in a mammal, including in a human, a disease or condition selected from Alzheimer's disease, hereditary cerebral hemorrhage with amyloidosis, cerebral amyloid angiopathy, a prion-mediated disease, inclusion body myositis, stroke, and Down's Syndrome, which method comprises administering to said mammal an amount of a compound of Formula I effective in treating said disease or condition.

Compounds in Formula I may be used alone or used as a combination with any other drug, including, but not limited to, any memory enhancement agent, antidepressant agent, anxiolytic, antipsychotic agent, sleep disorder agent, anti-inflammatory agent, anti-oxidant agent, cholesterol modulating agent (for example, an agent that lowers LDL or increases HDL), or anti-hypertension agent. Accordingly, this invention also provides a pharmaceutical composition for treatment of a mammal, including a human, in need thereof comprising an effective amount of a compound of Formula I and an effective amount of another drug, for example a memory enhancement agent, antidepressant agent, anxiolytic, antipsychotic agent, sleep disorder agent, anti-inflammatory agent, anti-oxidant agent, cholesterol modulating agent (for example, an agent that lowers LDL or increases HDL), or anti-hypertension agent, and a pharmaceutically acceptable carrier. This invention also provides a method for treating dementia, for example Alzheimer's disease, in a mammal, including in a human, comprising administering to the mammal an effective amount of a compound of Formula I and an effective amount of another drug, for example a memory enhancement agent, antidepressant agent, anxiolytic, antipsychotic agent, sleep disorder agent, anti-inflammatory agent, anti-oxidant agent, cholesterol modulating agent (for example, an agent that lowers LDL or increases HDL), or anti-hypertension agent, wherein the compound of Formula I and the other drug are administered separately or together in a single pharmaceutical composition.

Compounds of Formula I, or any of the combinations described in the immediately preceding paragraph, may optionally be used in conjunction with a know P-glycoprotein inhibitor, such as verapamil.

References herein to diseases and conditions “associated with Aβ-peptide production” mean a disease or condition that is caused at least in part by Aβ-peptide and/or the production thereof. Thus, Aβ-peptide is a contributing factor, but not necessarily the only contributing factor, to “a disease or condition associated with Aβ-peptide production”.

The terms “treatment”, “treating”, and the like, refer to reversing, alleviating, or inhibiting the progress of a disorder or condition. As used herein, “treatment” and “treating” and like terms can also refer to decreasing the probability or incidence of occurrence of a disease or condition in a mammal compared to an untreated control population, or in the same mammal prior to treatment, according to the present invention. “Treatment” or “treating” can also include delaying or preventing the onset of a disease or condition. “Treatment” or “treating” as used herein also encompasses preventing the recurrence of disease or condition.

### DETAILED DESCRIPTION OF THE INVENTION

Compounds of Formula I may be prepared according to the following reaction Schemes and discussion. Unless otherwise indicated, R¹, R², R³, R⁴, R⁵, R⁶, R⁷, A, and Z in the reaction schemes and discussion that follows are as defined above.

The compounds of formula (I) may have asymmetric carbon atoms and may therefore exist as racemic mixtures, diasteroisomers, or as individual optical isomers.

Separation of a mixture of isomers of compounds of Formula I into single isomers may be accomplished according to conventional methods known in the art.

The compounds of the formula (I) can be prepared by the methods described below, together with synthetic methods known in the art of organic chemistry, or modifications and derivatisations that are familiar to those of ordinary skill in the art. Preferred methods include, but are not limited to, those described below.

The reactions below are performed in solvents appropriate to the reagents and materials employed and are suitable for use in the reactions. In the description of the synthetic methods described below, it is also to be understood that all proposed or performed reaction conditions, including choice of solvent, reaction temperature, reaction duration time, reaction pressure, reaction conditions (such as anhydrous conditions, under argon, under nitrogen, etc.), and work up procedures, are chosen to be the conditions standard for that reaction, which should be readily recognized by one skilled of art. Alternate methods may also be used.

2-amino-1,3-thiazoles II may be prepared by known methods (e. g., Can. J. Chem., EN, 66 (1988), 1617-1624; Chem. Heterocycl. Compd. (Engl. Transl.), EN, 5, (1969) 46-48; J. Org. Chem. USSR (Engl. Transl.), EN, 6, (1970), 1196-1200; Hoekfelt, B.; Joensson, A.; JMPCAS; J. Med. Pharm. Chem., EN, 5, (1962) 247-257.; J. Chem. Soc., (1951), 2430,2440; J. Amer. Chem. Soc., 72 (1950), 3722; J. Chem. Soc., (1945) 455, 457;) or by the methods described below. For example, compounds of formula II can be obtained by reacting a compound of formula VII, wherein L¹ is a leaving group such as a bromine, chlorine or iodine, with thiourea in a suitable solvent or a mixture of solvents, such as C₁-C₄ alcohol, THF, 1,4-dioxane, toluene, diethyl ether, DMF, water, methylene chloride, or chloroform, at a suitable temperature, such as from about 0° C. to about reflux.

Referring to Scheme 1, compounds of formula VII can be prepared by reacting compounds of formula VIII with halogen such as I₂, Br₂, Cl₂, N-Bromosuccinate (NBS), N-chlorosuccinate, or N-bromobarbiturate, with or without acetic acid, in an appropriate solvent, such as diethyl ether, THF, 1,4-dioxane, methylene chloride, dichloroethane, chloroform, carbon tetrachloride, or benzene, at a suitable temperature, for example from about −78° C. to about reflux, preferably at temperature from about −78° C. to about room temperature, using standard conditions or conditions analogous to those found in the literature.

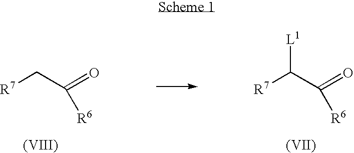

Alternatively, compounds of formula II may be prepared by reacting compounds analogous to compounds of formula II, but wherein R⁷ is H, with n-BuLi; quenching with an electrophile (such as trimethylsilyl chloride) to protect the free NH₂ group of the compounds analogous to formula II; then adding additional n-BuLi to generate a carbanion that is quenched with an electrophile (such as an aldehyde, ketone, alkyl halide, etc.); followed by acid/base work-up. This method is similar to methods described in the literature (Can. J. Chem., EN, 66 (1988), 1617-1624).

Compounds of formula II wherein R⁷ contains an alcohol moiety may be oxidized using standard oxidation method known in art, such as, e.g., Dess-Martin reagents, Swern oxidation, or use of CrO₃, to provide compounds of formula II wherein R⁷ is a ketone or aldehyde. Compounds of formula II wherein R⁷ is a ketone or aldehyde may convert to the corresponding compounds of formula II wherein R⁷is an imine (by reaction with an amine), olefin (by a Wittig reaction), alcohol (by a Grignard reaction), or other derivative (by standard reactions).

The compounds of formula I of the present invention and their salts can be prepared by a process comprising reacting a compound of formula II

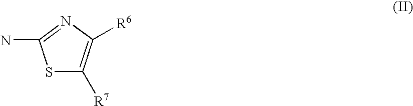

with a compound of formula III

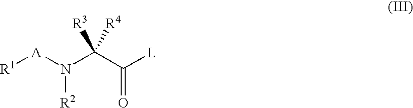

or reacting a compound of formula IV

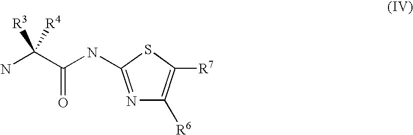

with a compound of formula V

R¹-A-L (V)

wherein R¹, R³, R⁴, R⁶, R⁷, and A are as defined above and L is hydroxy or a suitable leaving group. If desired, the 2-amino-1,3-thiazole derivative of formula I or synthetic intermediate of formula IV may be converted into a salt by methods known to those of ordinary skill in the art.

Examples of specific compounds of formula III and V wherein L is hydroxy or a suitable leaving group are those wherein L represents a halogen atom, such as Cl, Br, or I, or A-L is an alkyl or aryl ester.

Compounds in formula I can be prepared by reacting a compound of formula II and a carboxylic acid of formula III, or a compound of formula IV with a compound of formula V. Compounds of formula IV can be prepared by reacting a compound of formula II with a compound of formula VI.

The reaction between compounds of formula II and compounds of formula III, between compounds of formula IV and compounds of formula V, and between compounds of formula II and compounds of formula VI, can be carried out by standard methods. For example, wherein L is a hydroxy group, these reactions can be carried out in the presence of a coupling agent or a polymer supported coupling agent, such as, for example, carbodiimide, i.e. 1,3-dicyclohexylcarbodiimide (DCC), 1,3-diisopropylcarbodiimide, 1-(3-dimethylaminopropyl)-3-ethylcarbodiimide (EDC), N-cyclohexylcarbodiimide, or N′-methylpolystyrene in the presence or absence of HOBt, in a suitable solvent such as, for instance, a single solvent or a combination of several solvents selected from dichloromethane (CH₂Cl₂), chloroform (CHCl₃), tetrahydrofuran (THF), diethyl ether (Et₂O), 1,4-dioxane, acetonitrile, (CH₃CN), toluene, N,N-dimethylformamide (DMF), or dimethylsulfoxide (DMSO), at a suitable temperature such as from about −10° C. to about reflux, for a suitable time monitored by chromatography or LC-MS. An alternative method wherein L is OH is carried out by converting OH to a leaving group by reaction with oxalyl chloride, thionyl chloride or a mixed anhydride method, using an alkyl chloroformate, such as C₁-C₄ alkyl chloroformate, in the presence of a base such as triethylamine, N,N-diisopropylethylamine, pyridine, or dimethylaminopyridine, in a suitable solvent such as, for example, methylene chloride, chloroform, tetrahydrofuran (THF), toluene, diethyl ether, acetonitrile, 1,4-dioxane, n,N-dimethylformamide, dimethylsulfoxide (DMSO), N-methyl pyrrolidinone (NMP), or xylene, at a temperature of from about −30° C. to about room temperature.

Alternatively, aminothiazole coupling may be achieved as follows. A compound of formula I may be prepared by coupling an amino-thiazole II with III wherein C(═O)L is an ester, in the presence of trialkylaluminium preferably trimethylaluminum in an appropriate solvent such as methylene chloride, THF, dioxane, toluene, etc., at an appropriate temperature, such as from about room temperature to about reflux, or in a sealed reactor (such as sealed tube or inscrewed vials). Similarly, compound IV may be prepared by reacting an amino-thiazole II, triamethylaluminum and N-Boc of an a-amino acid ester, followed by removal of the Boc group using standard methods.

The protected amino compounds, such as a compound with an N-Boc group, of formula VI can be prepared by methods well known in the literature, for example the methods described in Theodora W. Greene's book “Protective Groups in Organic Synthesis”. Compounds of formula IV can be prepared in an analogous method as above by reacting compound of formula II with a compound of formula VI, followed by deblocking the P¹ group. Deprotection can be performed by well-known methods, for example when P¹ is N-Boc, removal by any methods well-known in the literature, for example HCl(g) in an appropriate solvent such as 1,4-dioxane, diethylether or trifluoroacetic acid in methylene chloride. Many other amino protecting groups are known and may also be used, such as benzyl or p-methoxy-benzyl, trimethylsilyl, t-butyldimethylsilyl, etc. Methods for deblocking such groups are also well-known in the literature and may be used.

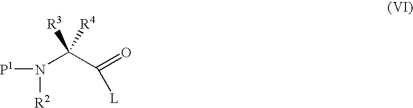

The compounds of formula II, III, and IV are known compounds or can be obtained according to known methods.

Compounds of formula III and V, wherein L is a leaving group as defined above, can be obtained according to conventional methods from the corresponding carboxylic acids of formula III where X is hydroxy.

Compounds of formula IV can be prepared by reacting a compound of formula II with a compound of formula V using known methods.

An ester group of R⁷ in compounds of formula I or II may be converted to the corresponding amide using a similar method for amide bond formation, preferably employing trimethylaluminum in an appropriate solvent or a mixture of solvents, such as THF/toluene.

A keto group of R⁷ in compounds of formula I or II may be converted to the corresponding amine using a well-established reductive amination method by reacting such ketone with an appropriate amine, with or without acid catalyst/ammonium acetate/dry agents (such as anhydrous Na₂SO₄ or MgSO₄), and a reducing agent, such as sodium triacetoxy borohydride, sodium cyanoborohydride, or sodium borohydride, or the corresponding polymer bound-NaBH₄, polymer bound-NaBH₃CN, or polymer bound-NaB(OAc)₃H, or any reducing agent (e.g., hydrogenation) that is known in the literature for reducing an imine bond to an amine, in an appropriate solvent, such as dichloroethane, chloroform, THF, MeOH, ethanol, isopropanol, t-butanol or toluene, at a temperature from about room temperature to about reflux, preferably from about room temperature to about 65° C.

Compounds wherein R⁶ is a halo group may be generated by reacting the starting material wherein R⁶ is H with NBS, NCS, or SO₂Cl₂, I₂ in an appropriate solvent such as methylene chloride or chloroform. The halo group may then be replaced with another group using methods known in the art, such as halogen-metal exchange, followed by quenching with an electrophile, or using typical Suzuki coupling conditions employing a catalyst such as a palladium complex, e.g., tetrakis(triphenylphosphine)-palladium, with sodium carbonate as a base, in a suitable solvent such as THF, DME, or ethanol, and a boronic acid.

The starting materials used in the procedures of the above reactions, the syntheses of which are not described above, are either commercially available, known in the art or readily obtainable from known compounds using methods that will be apparent to those skilled in the art.

The compounds of Formula I, and the intermediates shown in the above reaction schemes, may be isolated and purified by conventional procedures, such as recrystallization or chromatographic separation, such as on silica gel, either with an ethyl acetate/hexane elution gradient, a methylene chloride/methanol elution gradient, or a chloroform/methanol elution gradient. Alternatively, a reverse phase preparative HPLC or chiral HPLC separation technique may be used. In each of the reactions discussed or illustrated above, pressure is not critical unless otherwise indicated. Pressures from about 0.5 atmospheres to about 5 atmospheres is generally acceptable, and ambient pressure, i.e., about 1 atmosphere, is preferred as a matter of convenience.

Pharmaceutically acceptable salts of a compound of Formula I can be prepared in a conventional manner by treating a solution or suspension of the corresponding free base or acid with one chemical equivalent of a pharmaceutically acceptable acid or base. Conventional concentration or crystallization techniques can be employed to isolate the salts. Illustrative of suitable acids are acetic, lactic, succinic, maleic, tartaric, citric, gluconic, ascorbic, benzoic, cinnamic, fumaric, sulfuric, phosphoric, hydrochloric, hydrobromic, hydroiodic, sulfamic, sulfonic acids such as methanesulfonic, benzene sulfonic, p-toluenesulfonic, and related acids. Illustrative bases are sodium, potassium, and calcium.

A compound of this invention may be administered alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed by combining a compound of Formula I or a pharmaceutically acceptable salt thereof can then be readily administered in a variety of dosage forms such as tablets, powders, lozenges, syrups, injectable solutions and the like. These pharmaceutical compositions can, if desired, contain additional ingredients such as flavorings, binders, excipients and the like. Thus, for purposes of oral administration, tablets containing various excipients such as sodium citrate, calcium carbonate and calcium phosphate may be employed along with various disintegrants such as starch, methylcellulose, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof.

For parenteral administration, solutions containing a compound of this invention or a pharmaceutically acceptable salt thereof in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The sterile aqueous media employed are all readily available by standard techniques known to those skilled in the art.

A compound of Formula I or a pharmaceutically acceptable salt thereof can be administered orally, transdermally (e.g., through the use of a patch), parenterally (e.g. intravenously), rectally, or topically. In general, the daily dosage for treating a neurodegenerative disease or condition or a disease or condition associated with Aβ-peptide production will generally range from about 0.1 mg/kg to about 5 gm/kg body weight, preferably from about 0.1 mg/kg to about 100 mg/kg body weight. Variations based on the aforementioned dosage range may be made by a physician of ordinary skill taking into account known considerations such as the weight, age, and condition of the person being treated, the severity of the affliction, and the particular route of administration chosen.

A specific compound of Formula I can be determined to inhibit Aβ-peptide production using biological assays known to those of ordinary skill in the art, for example the assays described below.

The activity of compounds of the invention in inhibiting gamma-secretase activity was determined in a solubilized membrane preparation generally according to the description provided in McLendon et al. Cell-free assays for γ-secretase activity, The FASEB Journal (Vol. 14, December 2000, pp. 2383-2386). Using such assay, compounds of the invention were determined to have an IC₅₀ activity for inhibiting gamma-secretase activity of less than about 32 micromolar. For example, Example 11, below, had an IC₅₀ of about 5 micromolar.

The following Examples illustrate the present invention. It is to be understood, however, that the invention, as fully described herein and as recited in the claims, is not intended to be limited by the details of the following Examples.

### EXAMPLES

General Procedure A:

Coupling Method for Amide Formation

### a) EDC/HOBt/Trialkylamine Coupling Procedure

A mixture of a carboxylic acid (1.0 e.q.), amine (1.0 e.q.), HOBt (1.1-1.5 eq.), EDC (1.2-1.8 eq.) and a trialkylamine (triethylamine or diisopropylethylamine) (3-6 eq.) in an appropriate solvent or a mixture of solvents, for example methylene chloride, dichloroethane, THF, or DMF, was stirred at room temperature until product formation or disappearance of starting material. The solvent was removed under reduced pressure, the residue taken up in ethyl acetate (or similar selected solvent such as methylene chloride or chloroform) and water. The organic layer was separated, washed with dilute HCl (if the desired product contains a basic functional group, washing with dilute HCl may be omitted), brine, and dried over sodium sulfate. The solvent was then removed at reduced pressure to provide product.

### b) HATU/Trialkylamine Coupling Procedure

A mixture of a carboxylic acid (1.0 e.q.), amine (1.0 e.q.), HATU (1.1-1.5 eq.) and a trialkylamine (triethylamine or diisopropylethylamine) (3-6 eq.) in an appropriate solvent or a mixture of solvents, for example methylene chloride, dichloroethane, THF, or DMF, was stirred at room temperature until product formation or disappearance of starting material. The solvent was removed under reduced pressure, the residue taken up in ethyl acetate (or similar selected solvent such as methylene chloride or chloroform) and water. The organic layer was separated, washed with dilute HCl (if the desired product contains a basic functional group, washing with dilute HCl may be omitted), brine, and dried over sodium sulfate. The solvent was then removed at reduced pressure to provide product.

### c) PyBOP/Trialkylamine Coupling Procedure

A mixture of a carboxylic acid (1.0 e.q.), amine (1.0 e.q.), PyBOP (1.1-1.5 eq.) and a trialkylamine (triethylamine or diisopropylethylamine) (3-6 eq.) in an appropriate solvent or a mixture of solvents, for example methylene chloride, dichloroethane, THF, or DMF, was stirred at room temperature until product formation or disappearance of starting material. The solvent was removed under reduced pressure, the residue taken up in ethyl acetate (or similar selected solvent such as methylene chloride or chloroform) and water. The organic layer was separated, washed with dilute HCl (if the desired product contains a basic functional group, washing with dilute HCl may be omitted), brine, and dried over sodium sulfate. The solvent was removed at reduced pressure to provide product.

### d) HBTU/Trialkylamine Coupling Procedure

A mixture of a carboxylic acid (1.0 e.q.), amine (1.0 e.q.), HBTU (1.1-1.5 eq.), and a trialkylamine (triethylamine or diisopropylethylamine) (3-6 eq.) in an appropriate solvent or a mixture of solvents, for example methylene chloride, dichloroethane, THF, or DMF, was stirred at room temperature until product formation or disappearance of starting material. The solvent was removed under reduced pressure, the residue taken up in ethyl acetate (or similar selected solvent such as methylene chloride or chloroform) and water. The organic layer was separated, washed with dilute HCl (if the desired product contains a basic functional group, washing with dilute HCl may be omitted), brine, and dried over sodium sulfate. The solvent was removed at reduced pressure to provide product.

### e) Chloro-Alkylformate Coupling

A mixture of a carboxylic acid (1 eq.) and triethylamine (eq.) was dissolved in an appropriate solvent, such as DMF and cooled to −23° C. Iso-butyl formate (1 eq.) was added dropwise with stirring. After stirring for a period of time (form 15 min to 2 hr), a 2-amino-thiazole or an amine (1 eq.) was added and stirring continued for an additional 30 min at −23° C. The mixture was then warmed to room temperature until amide formation (typically overnight). The mixture was quenched with water and brine and extracted with an appropriate solvent such as ethyl acetate, methylene chloride or chlorform. The organic layer was washed with dilute NaHSO₄, NaHCO₃ and brine and the solvent was removed under reduced pressure to provide product. Purification may be necessary.

### f) Trimethylaluminum Coupling Procedure

A mixture of an amine or an amino-thiazole (1-2 eq.), 2M trimethylaluminum was made in an appropriate solvent, such as THF, toluene, xylene, methylene chloride, or dichloroethane, or a mixture of solvents such as THF/toluene. The mixture was stirred at room temperature for 15 min to 2 hr, then an ester (1 eq.) was added. The resulting mixture was stirred at temperature between room temperature to reflux until product formation. The mixture was carefully quenched with Rochelle salt and extracted with an appropriate solvent such as ethyl acetate or methylene chloride, filtered through celite. The organic layer was washed with dilute HCl, neutralized with saturated sodium bicarbonate, and washed with brine. The organic layer was separated, dried and concentrated to give the desired amide. Purification may be necessary.

General Procedure B:

Method for Reductive Amination

### a) Sodium Triacetoxyborohydride

An amine (1-4 eq.) in dichloroethane or THF was added to a solution of a ketone (1 eq.), NaBH(OAc)₃ (1-3 eq.) and acetic acid (1-3 eq.) in dichloroethane or THF. The mixture was stirred at room temperature until product formation or disappearance of starting material. The mixture was quenched with diluted base, extracted with methylene chloride or other appropriate solvent such as chloroform or ethyl acetate. The organic layer was separated, dried and concentrated to give the desired amide. Purification may be necessary.

### b) Sodium Cyanoborohydride

A mixture of a ketone or aldehyde (1 eq.), an amine (1-20 eq.), sodium cyanoborohydride (1-5 eq.), acetic acid (1-3 eq.), sodium acetate (1-3 eq.), anhydrous sodium sulfate in dichloroethane or THF was stirred at room temperature to 60° C., preferably heated at 35-50° C. until product formation. The mixture was quenched with diluted base, extracted with methylene chloride or other appropriate solvent such as chloroform or ethyl acetate. The organic layer was separated, dried and concentrated to give the desired amide. Purification may be necessary.

### c) Potassium Formate and Palladium Acetate

A solution of an aldehyde or a ketone (1 eq.) and an amine (1 eq.) in dry DMF was stirred at room temperature for 4 hr, in the presence of molecular sieves. To the resulting reaction mixture were added potassium formate (2 eq.) and palladium acetate (catalytic amount, 0.02 eq.). The mixture was heated at 40-60° C. to complete reaction (TLC) and after cooling it was diluted with ice-water. The mixture was extracted with an appropriate solvent (such as methylene chloride, ethyl acetate, or chloroform). The organic layer was separated, dried and concentrated to give the desired amide. Purification may be necessary.

General Procedure C:

Sodium Borohydride Reduction of Ketone or Aldehyde

A mixture of an aldehyde or a ketone (1 eq.) and sodium borohydride (1-10 eq.) in an appropriate solvent (methanol or ethanol) was stirred at 0° C. to room temperature for 10 minutes to complete reaction (TLC). The mixture was concentrated to a small volume, quenched with water, extracted with an appropriate solvent (such as methylene chloride, ethyl acetate, or chloroform). The organic layer was separated, dried and concentrated to give the desired amide. Purification may be necessary.

General Procedure D:

N-tBOC Deprotecting Procedure

To a solution of N-tBOC compound in 1,4-dioxane (0.03-0.09 M) was added 4 N HCl in 1,4-dioxane or anhydrous HCl gas under nitrogen. The reaction mixture was stirred at room temperature for 1-24 hrs until all the starting material consumed (TLC). The solution was concentrated and pumped in vacuo. The final HCl salt of the corresponding amine was typically used without further purification.

### Preparation A

### [1-(5-Methyl-thiazol-2-ylcarbamoyl)-butyl]-carbamic acid tert-butyl ester

A mixture of 2-tert-butoxycarbonylamino-pentanoic acid (1.0 eq.), 2-amino-5-methyl thiazole (1.0 eq.), HOBt (1.05 eq.), EDC.HCl (1.2 eq.) and a triethylamine (4 eq.) in methylene chloride was stirred at room temperature overnight. The mixture was quenched with water and extracted with methylene chloride. The organic layer was washed with diluted HCl, separated, dried over sodium sulfate and filtered. The solvent was removed at reduced pressure to provide product. M+1=314.3, ¹H NMR (DMSO-d6) d 7.11 (s,1H), 4.11(m,1H), 2.3(s,3H), 1.54(m,2H), 1.34(t,9H), 1.2-1.4(m,2H), 0.83(t,3H) ppm.

The following compounds were prepared by methods analogous to that described above for Preparation A:

{1-[5-(1-Ethyl-propyl)-thiazol-2-ylcarbamoyl]-butyl}-carbamic acid tert-butyl ester, M+1=370.4;

[1-(5-Isopropyl-thiazol-2-ylcarbamoyl)-butyl]-carbamic acid tert-butyl ester, M+1=342.5;

[1-(4,5-Dimethyl-thiazol-2-ylcarbamoyl)-butyl]-carbamic acid tert-butyl ester, M+1=328.4;

{1-[5-(5-Methoxy-1,5-dimethyl-hexyl)-thiazol-2-ylcarbamoyl]-butyl}-carbamic acid tert-butyl ester, M+1=442.5;

{1-[5-(5-Methoxy-1,5-dimethyl-hexyl)-thiazol-2-ylcarbamoyl]-ethyl}-carbamic acid tert-butyl ester, M+1=414.4;

[1-(4,5-Dimethyl-thiazol-2-ylcarbamoyl)-butyl]-carbamic acid tert-butyl ester, M+1=328.4;

[1-(Thiazol-2-ylcarbamoyl)-butyl]-carbamic acid tert-butyl ester; M+1=300.3;

2-(2-tert-Butoxycarbonylamino-butyrylamino)-4-trifluoromethyl-thiazole-5-carboxylic acid ethyl ester, M+1=426.3;

{1-[5-(5-Methoxy-1,5-dimethyl-hexyl)-thiazol-2-ylcarbamoyl]-propyl}-carbamic acid tert-butyl ester, M+1=428.3;

[1-(5-Isopropyl-thiazol-2-ylcarbamoyl)-ethyl]-carbamic acid tert-butyl ester, M+1=314.2;

[1-(5-Isopropyl-thiazol-2-ylcarbamoyl)-propyl]-carbamic acid tert-butyl ester, M+1=328.3;

[1-(5-Bromo-thiazol-2-ylcarbamoyl)-butyl]-carbamic acid tert-butyl ester, M+1=378.1, 380.0;

{1-[5-(5-Hydroxy-1,5-dimethyl-hexyl)-thiazol-2-ylcarbamoyl]-butyl}-carbamic acid tert-butyl ester, M+1=428.5;

{1-[5-(1,3,3-Trimethyl-butyl)-thiazol-2-ylcarbamoyl]-butyl}-carbamic acid tert-butyl ester, M+1=398.3;

(1-{5-[1-(3,3-Dimethyl-butylamino)-propyl]-thiazol-2-ylcarbamoyl}-butyl)-carbamic acid tert-butyl ester, M−1=439.6;

[1-(5-Propionyl-thiazol-2-ylcarbamoyl)-butyl]-carbamic acid tert-butyl ester, M+1=356.4;

{1-[5-(5-Methoxy-1,5-dimethyl-hexyl)-thiazol-2-ylcarbamoyl]-propyl}-carbamic acid tert-butyl ester, M+1=428.3;

### Preparation B

### 2-Amino-N-(5-isopropyl-thiazol-2-yl)-propionamide

4 N HCl in 1,4-dioxane (20 ml) was added to {1-[5-(5-methoxy-1,5-dimethyl-hexyl)-thiazol-2-ylcarbamoyl]-propyl}-carbamic acid tert-butyl ester (3.6 g, 8.43 ml) and stirred at room temperature for 20 min. The reaction solution was concentrated and pumped in vacuo to give the title compound (3.0 g, 98%) as a yellow oil.

The following compounds were prepared by methods analogous to that described above for Preparation B:

2-Amino-pentanoic acid (5-methyl-thiazol-2-yl)-amide;

2-Amino-pentanoic acid [5-(5-methoxy-1,5-dimethyl-hexyl)-thiazol-2-yl]-amide, M+1=270709;

2-Amino-pentanoic acid [5-(1-propyl-butyl)-thiazol-2-yl]-amide, M+1=298.4;

2-Amino-N-[5-(1-propyl-butyl)-thiazol-2-yl]-butyramide, M+1=284.3;

2-Amino-N-[5-(1-propyl-butyl)-thiazol-2-yl]-propionamide, M+1=270.3;

2-Amino-pentanoic acid [5-(1-ethyl-propyl)-thiazol-2-yl]-amide, M+1=270.3;

2-Amino-N-[5-(1-ethyl-propyl)-thiazol-2-yl]-propionamide;

2-Amino-N-[5-(1-ethyl-propyl)-thiazol-2-yl]-butyramide;

2-Amino-N-[5-(5-methoxy-1,5-dimethyl-hexyl)-thiazol-2-yl]-propionamide, M+1=314.3;

2-Amino-pentanoic acid (4,5-dimethyl-thiazol-2-yl)-amide, M+1=228.3;

2-Amino-pentanoic acid thiazol-2-ylamide, M+1=200.2;

2-(2-Amino-butyrylamino)-4-trifluoromethyl-thiazole-5-carboxylic acid ethyl ester, 1H NMR(CD3OD) d 4.39(q,2H), 4.10(m,1H), 2.0(m,2H), 1.38(t,3H), 1.07(t,3H) ppm;

2-Amino-N-[5-(5-methoxy-1,5-dimethyl-hexyl)-thiazol-2-yl]-butyramide M+1=328.4;

2-Amino-N-(5-isopropyl-thiazol-2-yl)-propionamide, M+1=214.2;

2-Amino-N-(5-isopropyl-thiazol-2-yl)-butyramide, M+1=228.2;

2-Amino-pentanoic acid (5-isopropyl-thiazol-2-yl)-amide, M+1=242.3;

2-Amino-pentanoic acid [5-(5-hydroxy-1,5-dimethyl-hexyl)-thiazol-2-yl]-amide, M+1=328.5;

### Example 1

### 2-[2-(3,5-Difluoro-phenyl)-acetylamino]-pentanoic acid (5-acetyl-thiazol-2-yl)-amide

A mixture of 2-[2-(3,5-Difluoro-phenyl)-acetylamino]-pentanoic acid (271 mg, 1 mmol), 5-acetyl-2-amino-thiazole (223 mg, 1 mmol), HOBt (165 mg, 1.2 mmol), EDC.HCl (290 mg, 1.5 mmol), and triethylamine (0.6 ml) in methylene chloride (20 ml) was stirred at room temperature overnight. The mixture was quenched with water, extracted with methylene chloride. The organic layer was washed with diluted HCl, separated, dried and concentrated. The residue was purified by Shimadzu HPL to provide the title compound as a yellow oil. LC-MS, RT 2.3 min, M+1=496.3.

### Example 2

### 2-[2-(3,5-Difluoro-phenyl)-acetylamino]-pentanoic acid (5-acetyl-4-methyl-thiazol-2-yl)-amide

To a solution of 5-acetyl-2-amino-4-methyl thiazole (2.19 g, 14.02 mmol) in a mixture of THF (10 ml) and toluene (20 ml) was added 2 M AlMe₃ in toluene (7 ml, 14 mmol) at room temperature and stirred for 1 hr. 2-[2-(3,5-Difluoro-phenyl)-acetylamino]-pentanoic acid methyl ester (2.000 g, 7.01 mmol) was added and the resulting mixture was heated at reflux overnight. The mixture was quenched with Rochelle salt and extracted with ethyl acetate. The organic layer was washed with water, diluted HCl, brine, separated, dried and concentrated to give 2.48 g of the title compound as an orange solid. The solid was purified by silica gel column chromatography using 1% methanol in methylene chloride as eluent to give the title compound as a yellow solid. LC-MS RT 2.3 min, M+1=410.3, ¹H NMR (CDCl₃) d 6.86(m,2H), 6.75(m,1H), 6.10(d,1H, NH), 4.68(m,1H), 3.65(Abq,2H), 2.64(s,3H), 2.50(s,3H), 1.89(m,1H), 1.68(m,1H), 1.34(m,2H), 0.92(t,3H) ppm.

### Example 3

### 2-(2-Hydroxy-3,3-dimethyl-butyrylamino)-pentanoic acid (5-formyl-thiazol-2-yl)-amide

A mixture of (S,S)-2-(2-hydroxy-3,3-dimethyl-butyrylamino)-pentanoic acid (1.09 g, 4.71 mmol), 2-amino-thiazole-5-carbaldehyde (0.606 g, 4.71 mmol), HOBt (0.763 g, 5.65 mmol), EDC.HCl (1.348 g, 7.07 mmol), and triethylamine (2.7 ml, 18.84 mmol) in methylene chloride (50 ml) was stirred at room temperature overnight. The mixture was quenched with water, extracted with methylene chloride. The organic layer was washed with diluted HCl, separated, dried and concentrated. The residue was purified by silica gel column chromatography using 2% methanol in methylene chloride as eluent to give the title compound (505 mg) as a yellow solid.

### Example 4

### 2-(2-Hydroxy-2-phenyl-acetylamino)-pentanoic acid [5-(2,2,2-trifluoro-1-hydroxy-ethyl)-thiazol-2-yl]-amide

To a solution of 2-(2-Hydroxy-2-phenyl-acetylamino)-pentanoic acid (5-trifluoroacetyl-thiazol-2-yl)-amide (43 mg) in methanol (2 ml) was added sodium borohydride (43 mg) at room temperature and stirred for 10 min. The mixture was quenched with water, concentrated to a small volume and extracted with methylene chloride. The organic layer was separated, dried, filtered, and concentrated to give the title compound (47 mg) which was purified by HPLC to give a white solid (18 mg) as a mixture of two isomers.

### Example 5

### 2-[2-(3,5-Difluoro-phenyl)-acetylamino]-pentanoic acid [4-methyl-5-(1-propylamino-ethyl)-thiazol-2-yl]-amide

A mixture of 2-[2-(3,5-Difluoro-phenyl)-acetylamino]-pentanoic acid (5-acetyl-4-methyl-thiazol-2-yl)-amide (100 mg, 0.24 mmol), n-propyl amine (0.5 ml), sodium cyanoborohydride (100 mg, 1.59 mmol), acetic acid (0.1 ml), sodium acetate (100 mg), anhydrous sodium sulfate (100 mg) in dichloroethane was heated at 45° C. in an oil bath overnight. The mixture was quenched with water and extracted with methylene chloride. The organic layer was separated, dried and concentrated to give the title compound (217 mg) as an oil. The oil was purified by shimadzu HPLC to yield the title compound as a white solid (45 mg). LC-MS RT 1.6 min, M−1=451, ¹H NMR (DMSO-d6) d 8.5 (m,1H), 7.07(m,1H), 6.97(m,2H), 5.87(brs,1H), 4.38(m,1H), 4.02(m,1H), 3.52(Abq,2H), 2.2-2.6(m,2H), 2.16(s,3H), 1.2-1.7(m,6H), 1.25(d,3H), 0.85(t,3H), 0.84(t,3H) ppm.

The following compounds were prepared by methods analogous to that described above for Example 5:

2-[2-(3,5-Difluoro-phenyl)-acetylamino]-pentanoic acid [5-(1-butylamino-ethyl)-4-methyl-thiazol-2-yl]-amide;

2-[2-(3,5-Difluoro-phenyl)-acetylamino]-pentanoic acid [4-methyl-5-(1-propylamino-ethyl)-thiazol-2-yl]-amide;

2-[2-(3,5-Difluoro-phenyl)-acetylamino]-pentanoic acid [4-phenyl-5-(piperidine-1-carbonyl)-thiazol-2-yl]-amide;

2-[2-(3,5-Difluoro-phenyl)-acetylamino]-pentanoic acid {5-[1-(2-methoxy-ethylamino)-ethyl]-4-methyl-thiazol-2-yl}-amide;

2-[2-(3,5-Difluoro-phenyl)-acetylamino]-pentanoic acid [4-methyl-5-(1-pyrrolidin-1-yl-ethyl)-thiazol-2-yl]-amide;

2-[2-(3,5-Difluoro-phenyl)-acetylamino]-pentanoic acid {5-[(1-benzyl-piperidin-4-ylamino)-methyl]-thiazol-2-yl}-amide;

2-[2-(3,5-Difluoro-phenyl)-acetylamino]-pentanoic acid [4-methyl-5-(1-methylamino-ethyl)-thiazol-2-yl]-amide;

2-[2-(3,5-Difluoro-phenyl)-acetylamino]-pentanoic acid [5-(1-ethylamino-ethyl)-4-methyl-thiazol-2-yl]-amide;

2-[2-(3,5-Difluoro-phenyl)-acetylamino]-pentanoic acid [5-(1-isopropylamino-ethyl)-4-methyl-thiazol-2-yl]-amide;

2-[2-(3,5-Difluoro-phenyl)-acetylamino]-pentanoic acid {5-[1-(2-hydroxy-ethylamino)-ethyl]-4-methyl-thiazol-2-yl}-amide;

2-[2-(3,5-Difluoro-phenyl)-acetylamino]-pentanoic acid [4-methyl-5-(1-morpholin-4-yl-ethyl)-thiazol-2-yl]-amide;

2-[2-(3,5-Difluoro-phenyl)-acetylamino]-pentanoic acid {4-methyl-5-[1-(4-methyl-piperazin-1-yl)-ethyl]-thiazol-2-yl}-amide;

2-[2-(3,5-Difluoro-phenyl)-acetylamino]-pentanoic acid (5-{[ethyl-(2-hydroxy-ethyl)-amino]-methyl}-thiazol-2-yl)-amide;

2-[2-(3,5-Difluoro-phenyl)-acetylamino]-pentanoic acid {5-[1-(3,3-dimethyl-butylamino)-ethyl]-4-methyl-thiazol-2-yl}-amide;

2-[2-(3,5-Difluoro-phenyl)-acetylamino]-pentanoic acid [5-(1-isobutylamino-ethyl)-4-methyl-thiazol-2-yl]-amide;

2-[2-(3,5-Difluoro-phenyl)-acetylamino]-pentanoic acid {4-methyl-5-[1-(3-methyl-butylamino)-ethyl]-thiazol-2-yl}-amide;

2-[2-(3,5-Difluoro-phenyl)-acetylamino]-pentanoic acid (5-morpholin-4-ylmethyl-thiazol-2-yl)-amide;

2-[2-(3,5-Difluoro-phenyl)-acetylamino]-pentanoic acid {5-[(butyl-ethyl-amino)-methyl]-thiazol-2-yl}-amide;

2-[2-(3,5-Difluoro-phenyl)-acetylamino]-pentanoic acid {5-[(1-ethyl-propylamino)-methyl]-thiazol-2-yl}-amide;

2-[2-(3,5-Difluoro-phenyl)-acetylamino]-pentanoic acid (5-ethylaminomethyl-thiazol-2-yl)-amide;

2-[2-(3,5-Difluoro-phenyl)-acetylamino]-pentanoic acid (5-dimethylaminomethyl-thiazol-2-yl)-amide;

2-[2-(3,5-Difluoro-phenyl)-acetylamino]-pentanoic acid [5-(isopropylamino-methyl)-thiazol-2-yl]-amide;

2-[2-(3,5-Difluoro-phenyl)-acetylamino]-pentanoic acid (5-aminomethyl-thiazol-2-yl)-amide;

2-(2-Hydroxy-3,3-dimethyl-butyrylamino)-pentanoic acid {5-[(3-methyl-butylamino)-methyl]-thiazol-2-yl}-amide;

2-(2-Hydroxy-3,3-dimethyl-butyrylamino)-pentanoic acid {5-[(3,3-dimethyl-butylamino)-methyl]-thiazol-2-yl}-amide;

2-(2-Hydroxy-3,3-dimethyl-butyrylamino)-pentanoic acid [5-(isobutylamino-methyl)-thiazol-2-yl]-amide;

2-(2-Hydroxy-3,3-dimethyl-butyrylamino)-pentanoic acid (5-{[methyl-(3-methyl-butyl)-amino]-methyl}-thiazol-2-yl)-amide;

2-[2-(3,5-Difluoro-phenyl)-acetylamino]-pentanoic acid [4-methyl-5-(1-phenethylamino-ethyl)-thiazol-2-yl]-amide;

2-[2-(3,5-Difluoro-phenyl)-acetylamino]-pentanoic acid [5-(1-benzylamino-ethyl)-4-methyl-thiazol-2-yl]-amide;

2-[2-(3,5-Difluoro-phenyl)-acetylamino]-pentanoic acid {4-methyl-5-[1-(2,2,2-trifluoro-ethylamino)-ethyl]-thiazol-2-yl}-amide;

2-[2-(3,5-Difluoro-phenyl)-acetylamino]-pentanoic acid [5-(1-dimethylamino-ethyl)-4-methyl-thiazol-2-yl]-amide;

2-[2-(3,5-Difluoro-phenyl)-acetylamino]-pentanoic acid {5-[1-(2-hydroxy-ethylamino)-ethyl]-4-methyl-thiazol-2-yl}-amide;

2-[2-(3,5-Difluoro-phenyl)-acetylamino]-pentanoic acid [5-(1-isobutylamino-ethyl)-4-methyl-thiazol-2-yl]-amide;

2-[(2-{2-[2-(3,5-Difluoro-phenyl)-acetylamino]-pentanoylamino}-thiazol-5-ylmethyl)-amino]-pentanoic acid methyl ester;

2-[2-(3,5-Difluoro-phenyl)-acetylamino]-pentanoic acid [5-(1-isopropylamino-ethyl)-thiazol-2-yl]-amide;

2-[2-(3,5-Difluoro-phenyl)-acetylamino]-pentanoic acid [5-(1-butylamino-ethyl)-thiazol-2-yl]-amide;

2-[2-(3,5-Difluoro-phenyl)-acetylamino]-pentanoic acid [5-(1-benzylamino-ethyl)-thiazol-2-yl]-amide;

2-[2-(3,5-Difluoro-phenyl)-acetylamino]-pentanoic acid {5-[1-(3,3-dimethyl-butylamino)-ethyl]-thiazol-2-yl}-amide;

2-[2-(3,5-Difluoro-phenyl)-acetylamino]-pentanoic acid {5-[1-(3-methyl-butylamino)-ethyl]-thiazol-2-yl}-amide;

2-[2-(3,5-Difluoro-phenyl)-acetylamino]-pentanoic acid [4-methyl-5-(1-methylamino-ethyl)-thiazol-2-yl]-amide;

2-[2-(3,5-Difluoro-phenyl)-acetylamino]-pentanoic acid [4-methyl-5-(1-methylamino-ethyl)-thiazol-2-yl]-amide;

2-[(2-{2-[2-(3,5-Difluoro-phenyl)-acetylamino]-pentanoylamino}-thiazol-5-ylmethyl)-amino]-pentanoic acid;

b 2-[2-(3,5-Difluoro-phenyl)-acetylamino]-pentanoic acid {5-[1-(2-hydroxy-ethylamino)-ethyl]-thiazol-2-yl}-amide;

2-[2-(3,5-Difluoro-phenyl)-acetylamino]-pentanoic acid [5-(1-phenethylamino-ethyl)-thiazol-2-yl]-amide;

2-[2-(3,5-Difluoro-phenyl)-acetylamino]-pentanoic acid [5-(1-morpholin-4-yl-ethyl)-thiazol-2-yl]-amide;

2-[2-(3,5-Difluoro-phenyl)-acetylamino]-pentanoic acid {5-[1-(1-benzyl-pyrrolidin-3-ylamino)-ethyl]-thiazol-2-yl}-amide;

2-[2-(3,5-Difluoro-phenyl)-acetylamino]-pentanoic acid {5-[1-(2-methoxy-ethylamino)-ethyl]-thiazol-2-yl}-amide;

2-[2-(3,5-Difluoro-phenyl)-acetylamino]-pentanoic acid [5-(1-isobutylamino-ethyl)-4-methyl-thiazol-2-yl]-amide;

2-[2-(3,5-Difluoro-phenyl)-acetylamino]-pentanoic acid [5-(1-isobutylamino-ethyl)-4-methyl-thiazol-2-yl]-amide;

2-[2-(3,5-Difluoro-phenyl)-acetylamino]-pentanoic acid [5-(1-propylamino-ethyl)-thiazol-2-yl]-amide;

2-[2-(3,5-Difluoro-phenyl)-acetylamino]-pentanoic acid {5-[1-(3,3-dimethyl-butylamino)-2,2,2-trifluoro-ethyl]-thiazol-2-yl}-amide;

The following compounds listed in Table 1-3 below were prepared according to methods analogous to those described above.

The following Examples described in Tables 1-3 below were synthesized by methods analogous to those described above:

TABLE 1

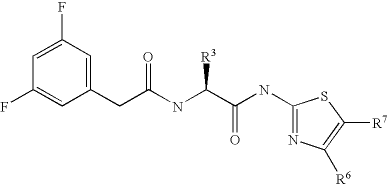

| | | | | | Mass | Mass |
|---|---|---|---|---|---|---|
| Example | R³ | R⁷ | R⁵ | MW | (M + 1) | (M − 1) |
| 6 | Me | COOEt | Ph | 365.4 | 474.3 | |
| 7 | n-Pr | H | COOEt | 439.5 | 440.2 | |
| 8 | n-Pr | S(O₂)-p-Ph-NO₂ | H | 538.6 | 539.2 | |
| 9 | n-Pr | S(O₂)-p-Ph-NHOH | H | 524.6 | 525.3 | |
| 10 | n-Pr | S(O₂)-p-Ph-NH₂ | H | 545 | 509.3 | |
| 11 | Et | 2-(5-Br-thieno) H | 500.4 | 502.2 | | |
| 12 | n-Pr | S(O₂)-p-Ph-NHCH₂Ph | H | 598.7 | 599.4 | |
| 13 | n-Pr | Me | H | 367.4 | 368.2 | |
| 14 | n-Pr | Me | Me | 381.4 | 382.3 | |
| 15 | n-Pr | NO₂ | H | 398.4 | 399.3 | |
| 16 | n-Pr | H | H | 353.4 | 354.3 | |
| 17 | n-Pr | Cl | H | 387.8 | 388.2 | |
| 18 | n-Pr | H | Me | 367.4 | 368.3 | |
| 19 | n-Pr | H | CH₂COOH | 411.4 | 412.3 | |
| 20 | n-Pr | NH₂ | H | 368.4 | 369.2 | |
| 21 | n-Pr | S(O₂)-p-Ph-Cl | H | 528.0 | 528.3 | |
| 22 | n-Pr | C(Me)(CH₂)₃C(Me₂) | H | 481.6 | 482.4 | |
| | | (OMe) | | | | |
| 23 | Et | Cyclopentyl | H | 407.5 | 408.3 | |
| 24 | Et | SMe | H | 385.5 | 386.3 | |
| 25 | Et | i-Pr | H | 381.4 | 382.5 | |
| 26 | n-Pr | H | CH₂CON(Et) | 494.6 | 495.5 | |
| | | | (n-Bu) | | | |
| 27 | n-Pr | H | CH₂CONHCH₂Ph | 500.6 | 501.4 | |
| 28 | n-Pr | Br | H | 432.3 | 432.2, | |
| | | | | | 434.2 | |
| 29 | Et | Ph | Ph | 491.6 | 214.2 | |
| 30 | Et | C(═O)Me | H | 381.4 | 382.5 | |
| 31 | Et | H | CH₂CONHEt | 438.5 | 439.3 | |
| 32 | Et | CH(Me)(Et) | H | 395.5 | 396.5 | |
| 33 | n-Pr | H | CH₂CONHCH₂- | 464.5 | 465.4 | |
| | | | cyclopropyl | | | |
| 34 | Et | H | C(Me)₂(COOEt) | 453.5 | 454.4 | |
| 35 | Et | p-Ph-F | H | 433.5 | 433.5 | |
| 36 | n-Pr | i-Pr | H | 395.5 | 396.4 | |
| 37 | n-Pr | COOEt | CH₂OEt | 483.5 | 484.2 | |
| 38 | n-Pr | CONH_{2 H} | 396.4 | 397.5 | | |
| 39 | n-Pr | NMePh | H | 458.5 | 459.5 | |
| 40 | n-Pr | C(═O)N-p-Ph-Cl | H | 521 | 521.6 | |
| 41 | n-Pr | H | CH₂COOEt | | | |
| 42 | Et | H | CH(═NOMe)(CO | 468.5 | 469.4 | |
| | | | OEt) | | | |
| 43 | n-Pr | COOMe | H | 411.4 | 412.3 | |
| 44 | n-Pr | COMe | H | 395.4 | 396.3 | |
| 45 | n-Pr | CH(Me)[CH₂CH₂CH₂C | H | 511.6 | 512.6 | |
| | | Me₂(OMe)] | | | | |
| 46 | Et | CH(Me)(CH₂CH₂CH═ | H | 449.6 | 450.4 | |
| | | CMe₂) | | | | |
| 47 | n-Bu | i-Pr | H | | | |
| 48 | Me | CH(Me)[CH₂CH₂CH₂C | H | 453.6 | 454.4 | |
| | | Me₂(OH)] | | | | |
| 49 | Me | CH(Me)[CH₂CH₂CH₂C | H | 467.6 | 468.4 | |
| | | Me₂(OMe)] | | | | |
| 50 | n-Pr | CH(Me)(CH₂CMe₃) | H | 451.6 | 452.4 | |
| 51 | n-Pr | CMe₂(OH) | H | 411.5 | 412.3 | |
| 52 | Et | CH(Me)[CH₂CH₂CH₂C | H | 481.2 | 482.2 | |
| | | Me₂(OMe)] | | | | |
| 53 | n-Pr | C(Et)₂(OH) | H | 439.5 | 440.3 | |
| 54 | Et | CH(Me)[CH₂CH₂CH₂C | H | 481.6 | 482.2 | |
| | | Me₂(OMe)] | | | | |
| 55 | Me | CH(Me)(CH₂CH₂CH═ | H | 435.5 | 436.3 | |
| | | CMe₂) | | | | |
| 56 | n-Pr | CH(Me)(NH-nBu) | H | 452.6 | 453.2 | |
| 57 | n-Pr | CHEt₂ | H | 423.5 | 424.3 | |
| 58 | n-Pr | CH(n-Pr)₂ | H | 451.6 | 452.3 | |
| 59 | n-Pr | CH(Et)(CH₂CHMe₂) | H | 451.6 | 452.2 | |
| 60 | n-Pr | CH(Me)(OH) | H | 397.5 | 398.3 | |
| 61 | n-Pr | COMe | Me | 409.45 | 410.3 | |
| 62 | n-Pr | CH(Me)(NH-n-Bu) | Me | 466.6 | 467.4 | |
| 63 | n-Pr | CH(Me)(NH-n-Pr) | Me | 452.6 | 453.4 | |
| 64 | n-Pr | Et | H | 381.5 | 382.0 | |
| 65 | n-Pr | CHO | H | 381.4 | 382.0 | |
| 66 | n-Pr | SEt | H | 413.5 | 414.9 | |
| 67 | n-Pr | CH₂SCH₂COOEt | H | 485.6 | 485.9 | |
| 68 | n-Pr | (S)—CH(Me)(OH) | Me | 411.5 | 412.1 | |
| 69 | n-Pr | (R)—CH(Me)(OH) | Me | 411.5 | 412.1 | |
| 70 | n-Pr | C(Et)(═CHMe) | H | 421.5 | 422.2 | |
| 71 | n-Pr | CH(Me)(NH- | Me | 468.6 | | 467.3 |
| | | CH₂CH₂OMe) | | | | |
| 72 | n-Pr | C(Me)(═CHMe) | H | 435.5 | 436.3 | |
| 73 | n-Pr | C(CH₂CHMe₂)(═CH₂) | H | 435.5 | 436.3 | |
| 74 | n-Pr | CH(Me)(NHMe) | Me | 424.5 | | 423.5 |
| 75 | n-Pr | CH(Me)(NHEt) | Me | 438.5 | | 437.5 |
| 76 | n-Pr | CH(Me)(NH-i-Pr) | Me | 452.6 | | 451.6 |
| 77 | n-Pr | CH(Me)(NHCH₂CH₂OH) | Me | 454.5 | | 453.4 |
| 78 | Me | CHEt₂ | H | 395.5 | 396.3 | |
| 79 | n-Pr | CH₂N(Et)(CH₂CH₂OH) | H | 454.5 | 455.4 | |
| 80 | n-Pr | C(Me)[NH(CH₂)₂CMe₃] | Me | 494.7 | 495.5 | |
| 81 | n-Pr | C(Me)(NHCH₂CHMe₂) | Me | 466.6 | | 465.3 |
| 82 | n-Pr | C(Me)[NH(CH₂)₂CHMe₂] | Me | 480.2 | 481.5 | |
| 83 | n-Pr | CH₂OH | H | 383.4 | 384.3 | |
| 84 | n-Pr | CH₂NEt(n-Bu) | H | 466.6 | | 465.3 |
| 85 | n-Pr | CH₂NHCHEt₂ | H | 452.6 | 453.4 | |
| 86 | n-Pr | CH₂NHEt | H | 410.5 | | 409.2 |
| 87 | n-Pr | CH₂NMe₂ | H | 410.5 | 411.3 | |
| 88 | n-Pr | CH₂NHCHMe₂ | H | 424.5 | 425.3 | |
| 89 | n-Pr | CH₂NH₂ | H | 382.4 | 381.3 | |
| 90 | n-Pr | CH(Me)(NHCH₂CH₂Ph) | Me | 514.6 | 515.4 | |
| 91 | n-Pr | CH(Me)(NHCH₂Ph) | Me | 500.6 | 501.5 | |
| 92 | n-Pr | COMe | H | 395.4 | 396.4 | |
| 93 | n-Pr | CH(Me)[CH₂CH₂CH₂C | H | 481 | 483.5 | |
| | | Me₂(OH)] | | | | |
| 94 | n-Pr | CH(Me)[CH₂CH₂CH₂C | H | 495.6 | 497.6 | |
| | | Me₂(OMe)] | | | | |
| 95 | n-Pr | CH(Me)(CH₂CH₂CH═ | H | 463.6 | 465.5 | |
| | | CMe₂) | | | | |
| 96 | n-Pr | CH(Me)(NHCH₂CF₃) | Me | 492.5 | 493.4 | |
| 97 | n-Pr | CH(Me)(NMe₂) | Me | 438.5 | | 437.4 |
| 98 | n-Pr | CH₂NHCH(n- | H | 496.6 | 497.3 | |
| | | Pr)(COOMe) | | | | |
| 99 | n-Pr | CH(Me)(NHCHMe₂) | H | 438.5 | 439.5 | |
| 100 | n-Pr | CH(Me)(NHCH₂Ph) | H | 486.6 | 487.5 | |
| 101 | n-Pr | C(Me)[NH(CH₂)₂CMe₃] | H | 480.6 | 481.6 | |
| 102 | n-Pr | C(Me)[NH(CH₂)₂CHMe₂] | H | 466.6 | 467.6 | |
| 103 | n-Pr | Et | Me | 395.5 | 396.5 | |
| 104 | n-Pr | (S)—CH(Me)(NHMe) | Me | 424.5 | | 423.3 |
| 105 | n-Pr | (R)—CH(Me)(NHMe) | Me | 424.5 | | 423.3 |
| 106 | n-Pr | CH₂NHCH(n- | H | | | |
| | | Pr)(COOH) | | | | |
| 107 | n-Pr | CH(Me)(NHCH₂CH₂OH) | H | 440.5 | 441.5 | |
| 108 | n-Pr | CH(Me)(NHCH₂CH₂Ph) | H | 500.6 | 501.5 | |
| 109 | n-Pr | C(═O)CF₃ | H | 449.4 | 450.3 | |
| 110 | n-Pr | CMe(OH)(CH₂CHOMe₂) | H | 485.5 | 486.3 | |
| 111 | n-Pr | CH(OH)(CF₃) | H | 451.4 | 452.3 | |
| 112 | n-Pr | CH(Me)(NHCH₂CH₂O | H | 454.5 | 455.5 | |
| | | Me) | | | | |
| 113 | n-Pr | (S)— | Me | 466.6 | 467.2 | |
| | | CH(Me)(NHCH₂CHMe₂) | | | | |
| 114 | n-Pr | (R)— | Me | 466.6 | 467.2 | |
| | | CH(Me)(NHCH₂CHMe₂) | | | | |
| 115 | n-Pr | C(Me)(NH-n-Pr) | H | 438.5 | 439.5 | |
| 116 | n-Pr | CH(CF₃)[NH(CH₂)₂CMe₃] | H | 534.6 | 535.4 | |

TABLE 2

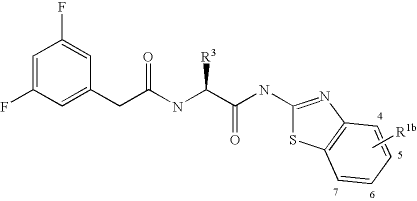

| | | | | Mass spectra |
|---|---|---|---|---|
| Example | R³ | R^{1b} | MW | (M + 1) |
| 117 | n-Pr | H | 403.5 | 404.3 |
| 118 | Et | 5-Me | 403.5 | 404.3 |
| 119 | Et | 5-OMe | 419.5 | 420.3 |
| 120 | Et | 5-Cl | 423.9 | 424.4 |
| 121 | Et | 7-Cl | 423.9 | 424.4 |

TABLE 3

| | | | Mass | Mass |
|---|---|---|---|---|
| Example | Structure | MW | (M + 1) | (M − 1) |
| 122 | 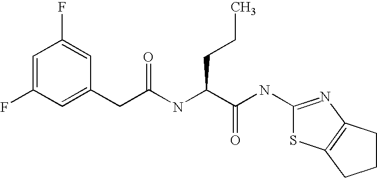 | 393.5 | 394.3 | |
| 123 | 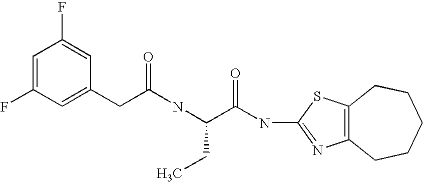 | 407.5 | 408.3 | |
| 124 | 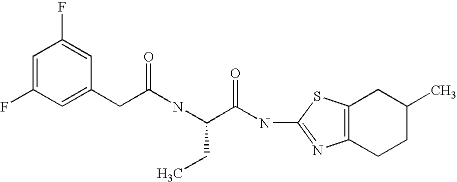 | 407.5 | 408.4 | |
| 125 | 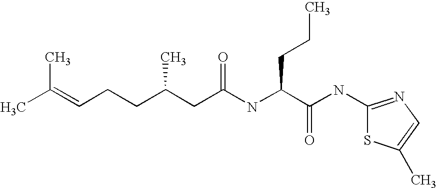 | 365.5 | 366.4 | |
| 126 | 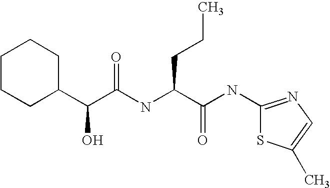 | 353.5 | 354.4 | |
| 127 | 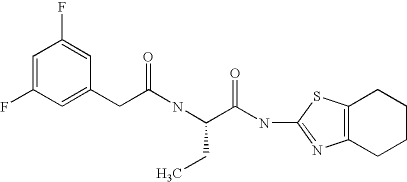 | 393.5 | 394.4 | |
| 128 | 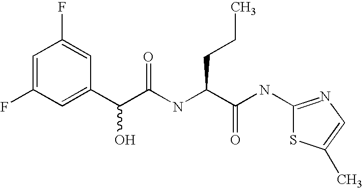 | 383.4 | 384.3 | |
| 129 | 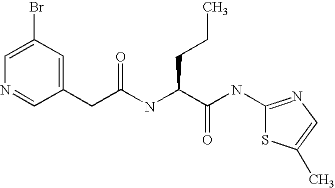 | 411.3 | 411.2, 413.2 | |
| 130 | 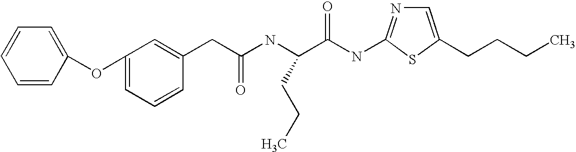 | 465.6 | 466.6 | |
| 131 | 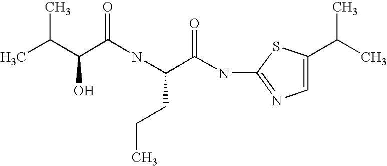 | 341.5 | 342.1 | |
| 132 | 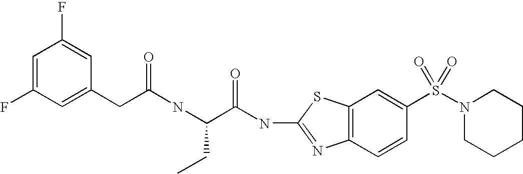 | 536.6 | 537.4 | |
| 133 | 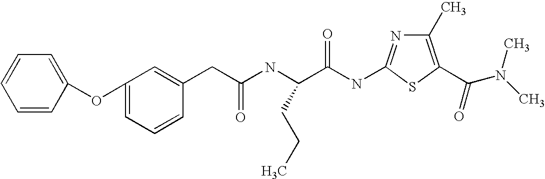 | 494.6 | 495.5 | |
| 134 | 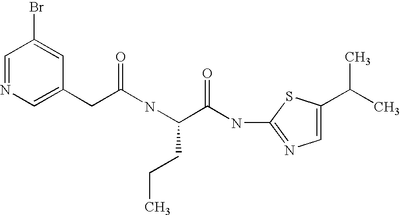 | 439.4 | 441.3 | |
| 135 | 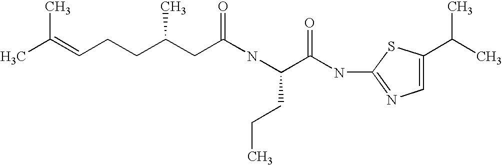 | 393.6 | 394.5 | |
| 136 | 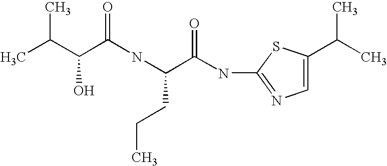 | 341.5 | 342.6 | |
| 137 | 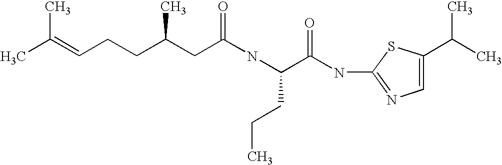 | 393.6 | 394.6 | |
| 138 | 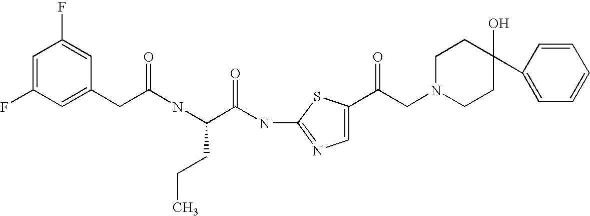 | 570.7 | 571.7 | |
| 139 | 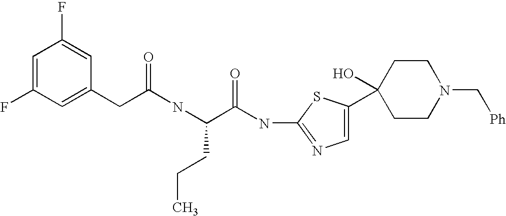 | 542.7 | 543.1 | |
| 140 | 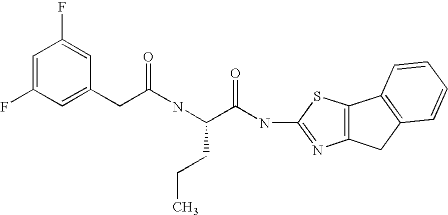 | 540.6 | 541.0 | |
| 141 | 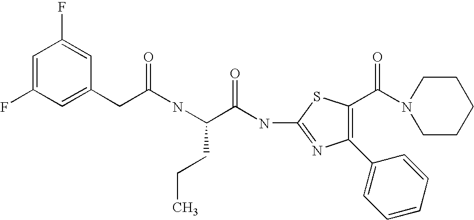 | 540.6 | 541.0 | |
| 142 | 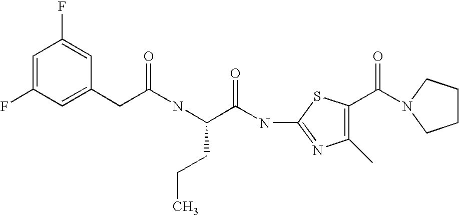 | 464.6 | | 465.4 |
| 143 | 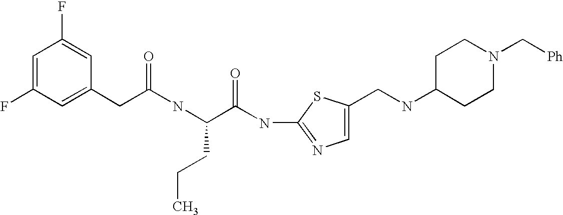 | 555.7 | 556.5 | |
| 144 | 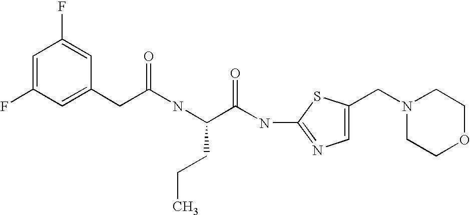 | 452.5 | 453.2 | |
| 145 | 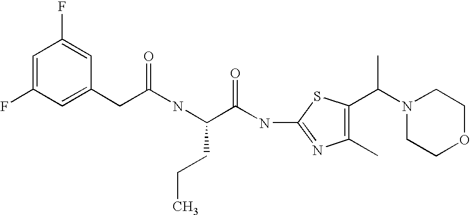 | 480.6 | | 479.6 |
| 146 | 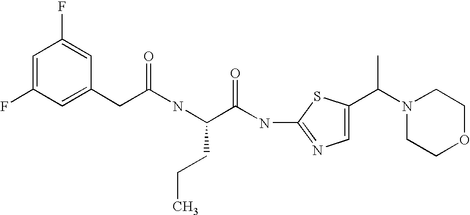 | 466.6 | 467.5 | |
| 147 | 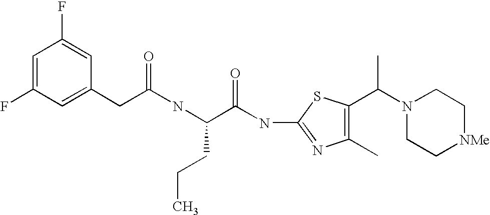 | 493.6 | | 492.6 |
| 148 | 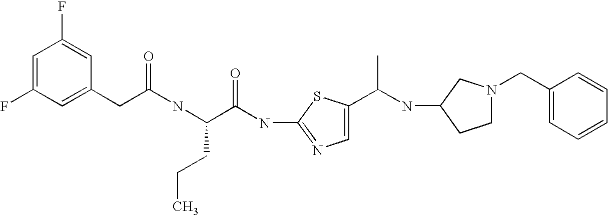 | 555.7 | 556.6 | |
| 149 | 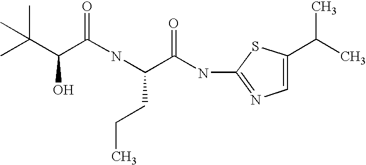 | 355.5 | 356.4 | |
| 150 | 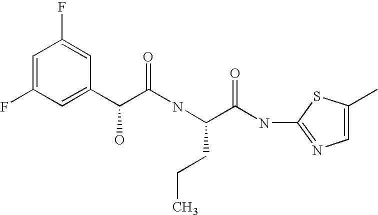 | 383.4 | 384.3 | |
| 151 | 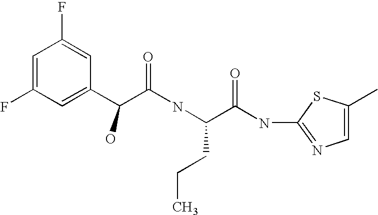 | 383.4 | 384.3 | |
| 152 | 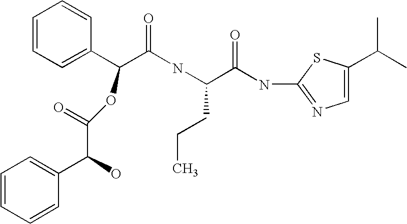 | 509.6 | 510.4 | |
| 153 | 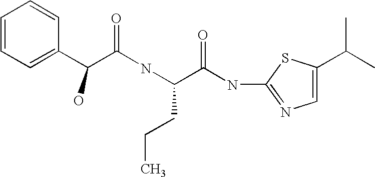 | 375.5 | 376.3 | |
| 154 | 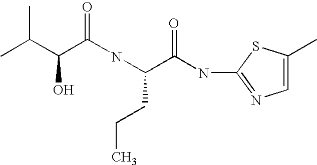 | 313.4 | 314.3 | |
| 155 | 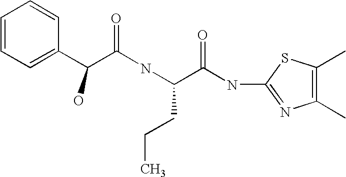 | 361.5 | 362.4 | |
| 156 | 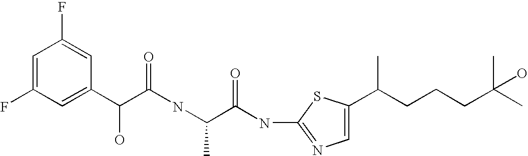 | 396.6 | 470.3 | |
| 157 | 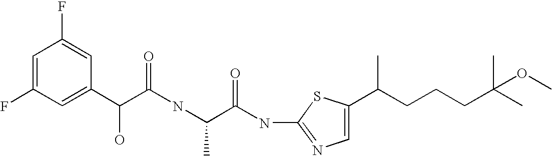 | 483.6 | 484.4 | |
| 158 | 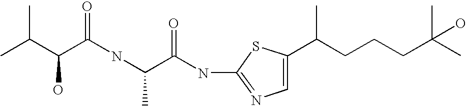 | 399.6 | 400.4 | |
| 159 | 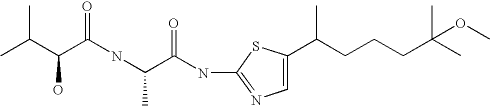 | 413.6 | 414.4 | |
| 160 | 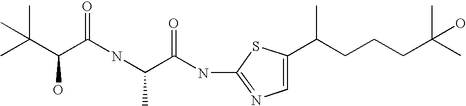 | 413.6 | 414.4 | |
| 161 | 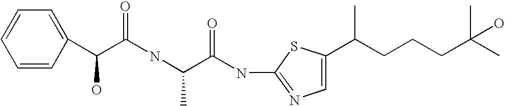 | 433.6 | 434.4 | |
| 162 | 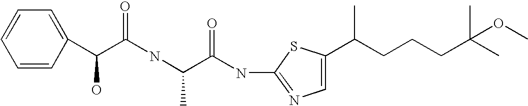 | 447.6 | 448.4 | |
| 163 | 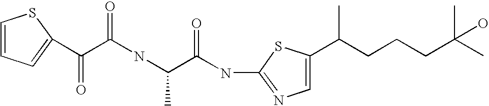 | 437.6 | 438.4 | |
| 164 | 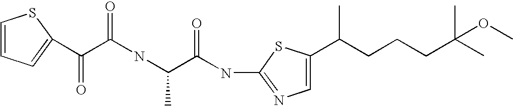 | 451.6 | 452.4 | |
| 165 | 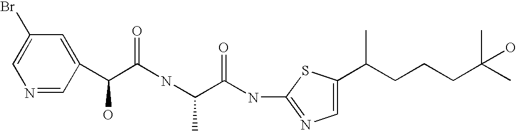 | 497.5 | 499.3 | |
| 166 | 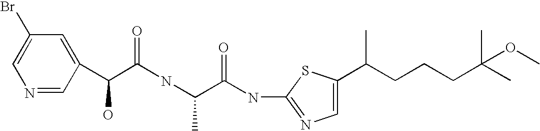 | 511.5 | 513.3 | |
| 167 | 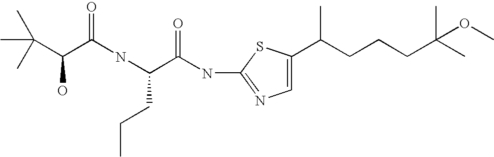 | 455.7 | 456.3 | |
| 168 | 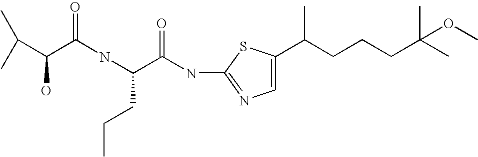 | 441.6 | 442.3 | |
| 169 | 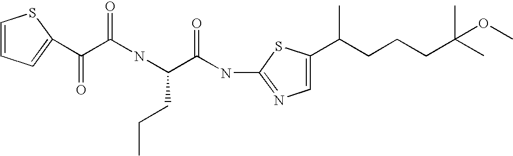 | 479.7 | 480.3 | |
| 170 | 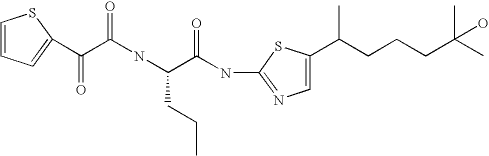 | 465.6 | 466.3 | |
| 171 | 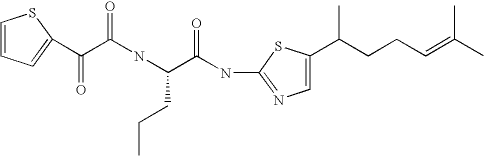 | 447.6 | 448.3 | |
| 172 | 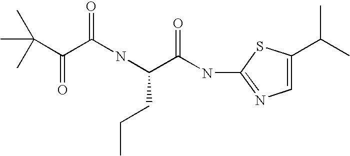 | 353.5 | 355.3 | |
| 173 | 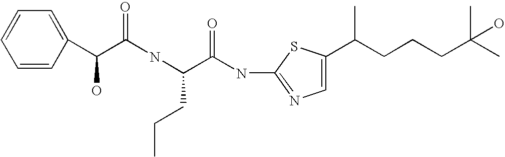 | 461.6 | 462.3 | |
| 174 | 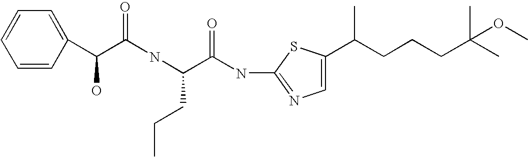 | 475.7 | 476.3 | |
| 175 | 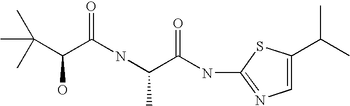 | 327.4 | 328.3 | |
| 176 | 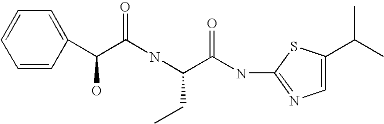 | 361.5 | 362.3 | |
| 177 | 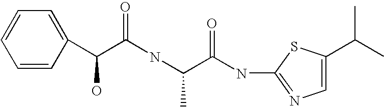 | 347.4 | 348.3 | |
| 178 | 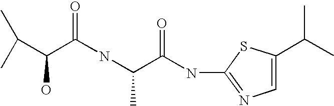 | 313.4 | 314.2 | |
| 179 | 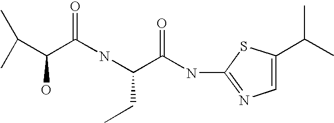 | 327.4 | 328.3 | |
| 180 | 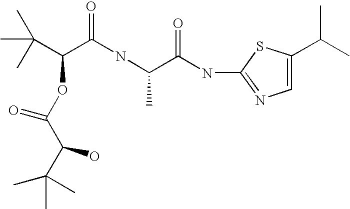 | 441.6 | 442.5 | |
| 181 | 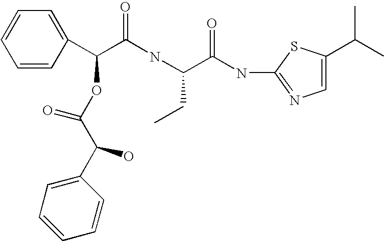 | 495.6 | 496.4 | |
| 182 | 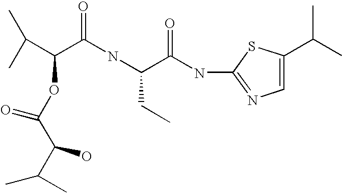 | 427.6 | 428.5 | |
| 183 | 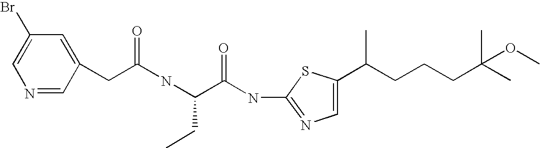 | 539.54 | 541.4 | |
| 184 | 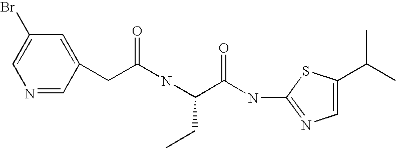 | 425.4 | 427.3 | |
| 185 | 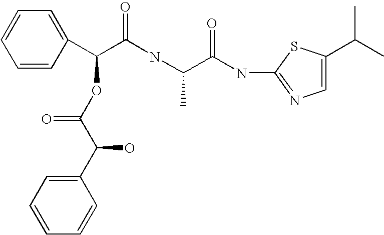 | 481.6 | 482.4 | |
| 186 | 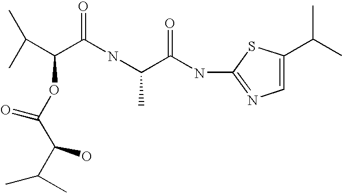 | 413.5 | 414.4 | |
| 187 | 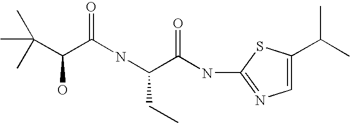 | 341.5 | 342.3 | |
| 188 | 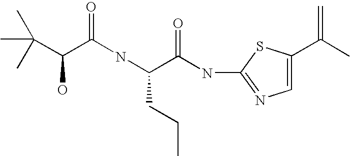 | 353.5 | 354.3 | |
| 189 | 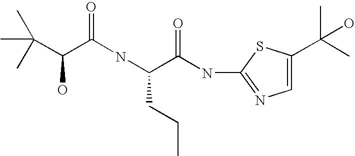 | 371.5 | 372.3 | |
| 190 | 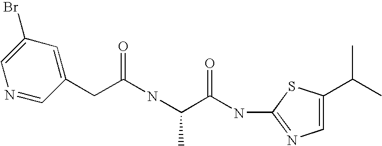 | 411.3 | 413.1 | |
| 191 | 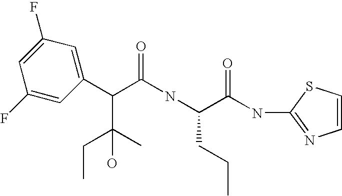 | 425.5 | 426.2 | |
| 192 | 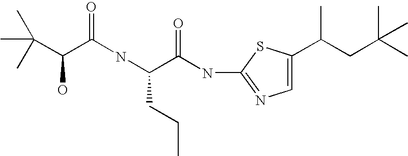 | 411.6 | 412.3 | |
| 193 | 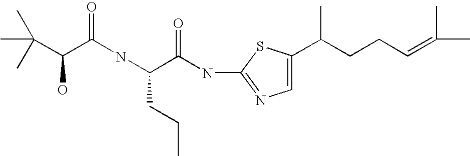 | 423.6 | 424.4 | |
| 194 | 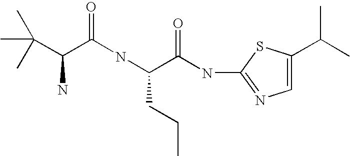 | 354.5 | 355.3 | |
| 195 | 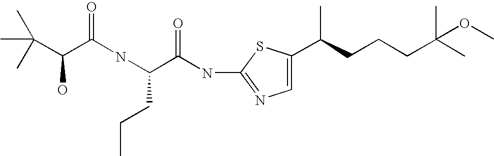 | 455.7 | 456.3 | |
| 196 | 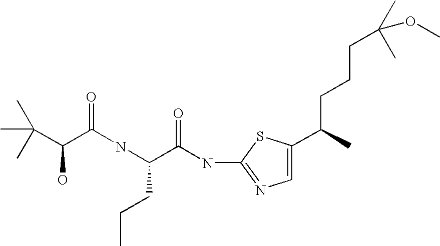 | 455.7 | 456.4 | |
| 197 | 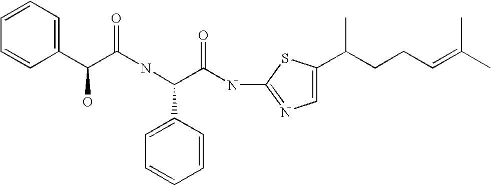 | 478.6 | 479.1 | |
| 198 | 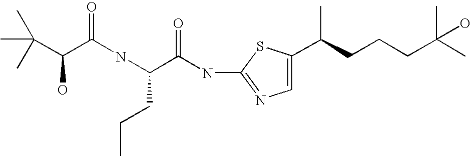 | 441.6 | 442.5 | |
| 199 | 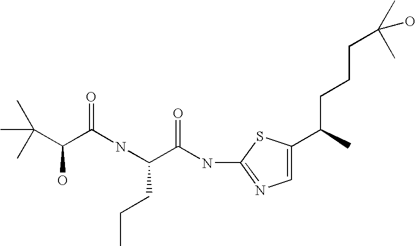 | 441.6 | 442.5 | |
| 200 | 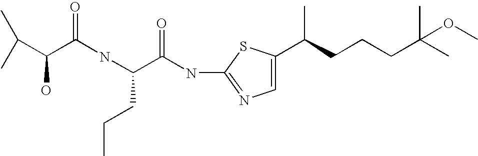 | 441.6 | 442.5 | |
| 201 | 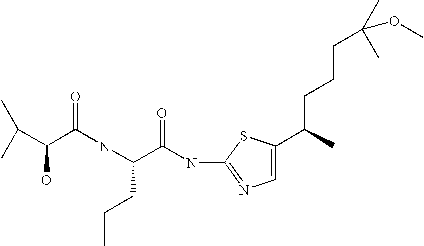 | 441.6 | 442.5 | |
| 202 | 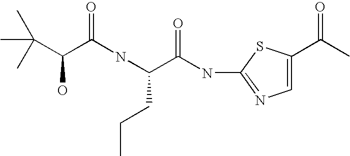 | 355.5 | 356.4 | |
| 203 | 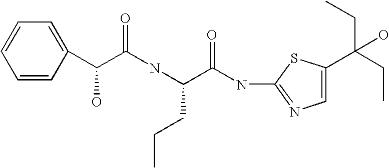 | 419.5 | 420.2 | |
| 204 | 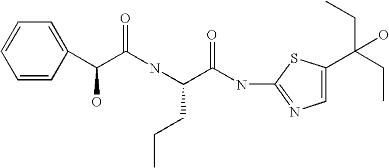 | 419.5 | 420.2 | |
| 205 | 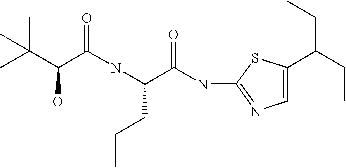 | 383.6 | 384.4 | |
| 206 | 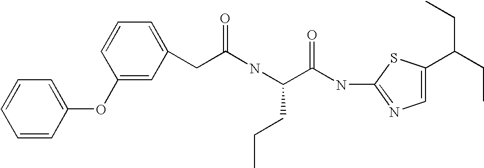 | 479.6 | 480.4 | |
| 207 | 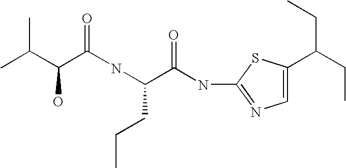 | 369.5 | 370.4 | |
| 208 | 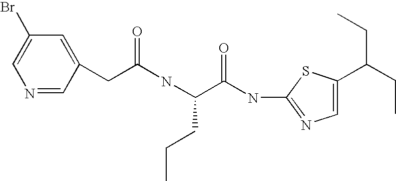 | 467.4 | 467.2, 469.2 | |
| 209 | 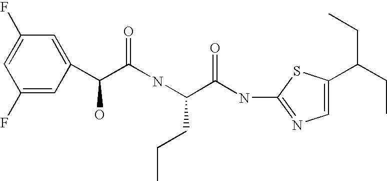 | 439.5 | 440.3 | |
| 210 | 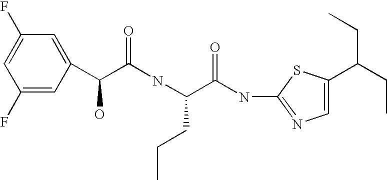 | 439.5 | 440.3 | |
| 211 | 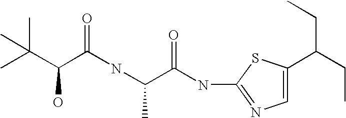 | 355.5 | 356.3 | |
| 212 | 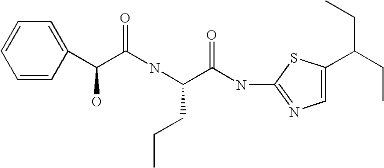 | 403.5 | 404.4 | |
| 213 | 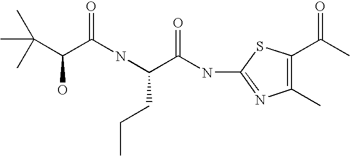 | 369.5 | 370.4 | |
| 214 | 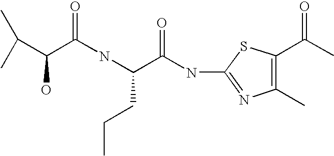 | 355.5 | | 354.3 |
| 215 | 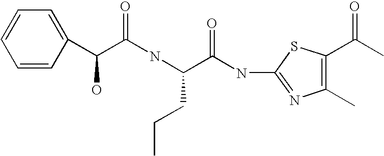 | 389.5 | 390.3 | |
| 216 | 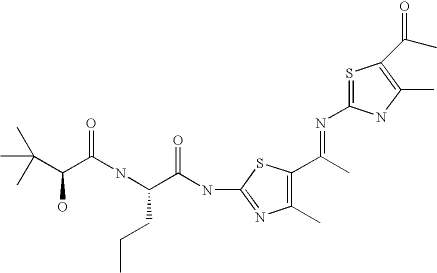 | 507.7 | 508.4 | |
| 217 | 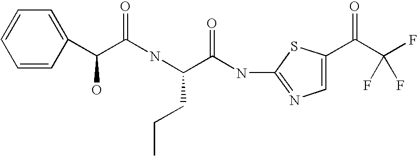 | 429.4 | 430.2 | |
| 218 | 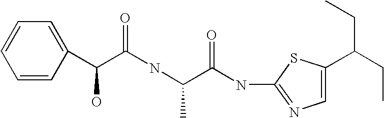 | 375.5 | 376.3 | |
| 219 | 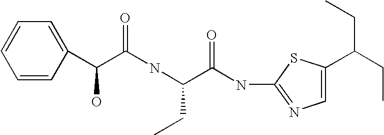 | 389.5 | 390.3 | |
| 220 | 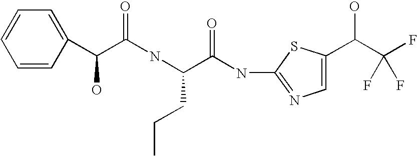 | 431.4 | 432.4 | |
| 221 | 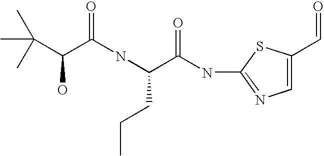 | 341.4 | 342.4 | |
| 222 | 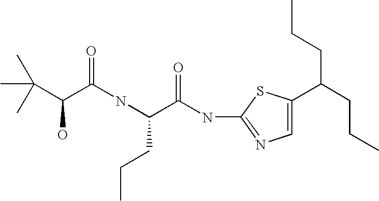 | 411.6 | 412.4 | |
| 223 | 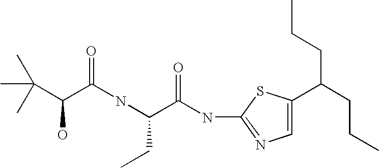 | 397.6 | 398.4 | |
| 224 | 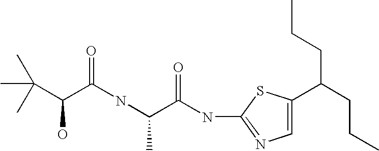 | 383.6 | 384.4 | |
| 225 | 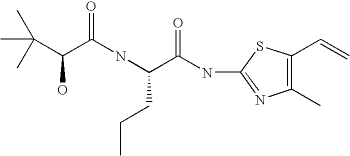 | 353.5 | 354.4 | |
| 226 | 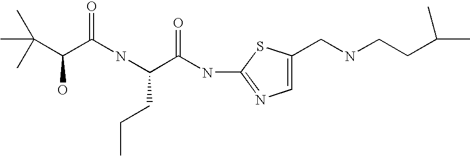 | 412.6 | 413.5 | |
| 227 | 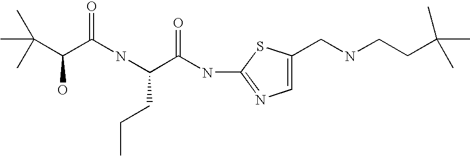 | 426.6 | 427.5 | |
| 228 | 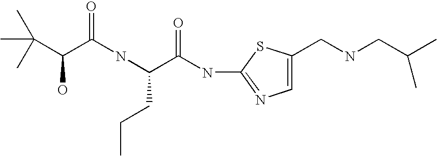 | 398.6 | 397.4 | |
| 229 | 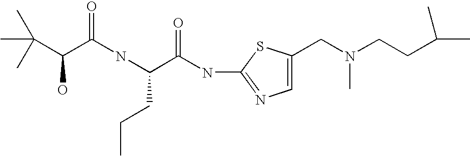 | 426.6 | 427.3 | |
| 230 | 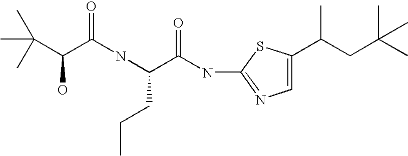 | 397.6 | 398.5 | |
| 231 | 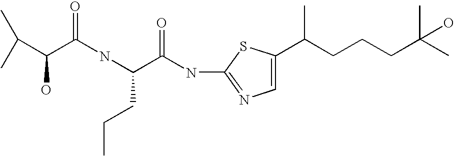 | 427.6 | 429.6 | 426.5 |
| 232 | 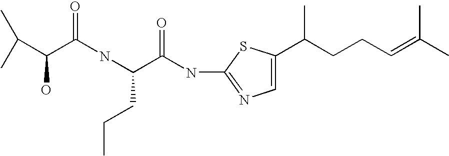 | 409.6 | 411.5 | |
| 233 | 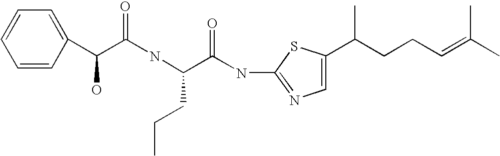 | 443.6 | 445.5 | |
| 234 | 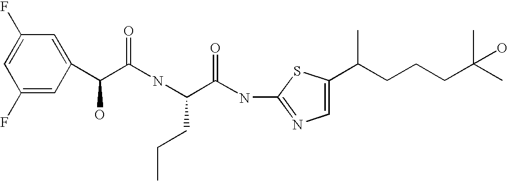 | 497.6 | 498.5 | |

What is claimed is:

1. A compound of Formula: 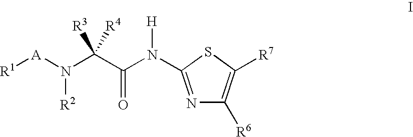
or a pharmaceutically acceptable salt thereof,
wherein:
A is selected from —C(═O)C(═O)—, —C(═O)NR⁹—, —C(═O)Z-, —C(═S)Z-, —C(═NR⁵)Z-, and —S(O)₂—;
wherein Z is —CH₂—, —CH(OH)—, —CH(OC(═O)R¹¹)—, —CH(NR⁹R¹⁰)—, —CH(CH₂(OH))—, —CH(CH(C₁-C₄ alkyl)(OH))—, or —CH(C(C₁-C₄ alkyl)(OH))—;
R¹ is selected from C₁-C₂₀ alkyl and —C₁-C₂₀ alkoxy, C₃-C₈ cycloalkyl, (C₄-C₈)cycloalkenyl, (C₅-C₁₁)bi- or tricycloalkyl, (C₇-C₁₁)bi- or tricycloalkenyl, (3-8 membered) heterocycloalkyl, (C₆-C₁₄)aryl, or (5-14 membered) heteroaryl, wherein said alkyl and alkoxy each optionally contains from one to five double or triple bonds, and wherein each hydrogen atom of said alkyl and alkoxy is optionally replaced with a fluorine;
wherein when R¹ is alkyl or alkoxy, R¹ is optionally substituted with from one to three substituents R^{1a}, and wherein when R¹ is cycloalkyl, cycloalkenyl, bi- or tricycloalkyl, bi- or tricycloalkenyl, heterocycloalkyl, aryl, or heteroaryl, then R¹ is optionally substituted with from one to three substituents R^{1b};
R^{1a} is in each instance independently selected from —OH, —C₁-C₆ alkyl independently optionally containing from one to three double or triple bonds, —C₁-C₆ alkoxy independently optionally containing from one to three double or triple bonds, —Cl, —F, —Br, —I, —CN, —NO₂, —NR⁹R¹⁰, —C(═O)NR⁹R¹⁰, —S(O)ₙNR⁹R¹⁰, —C(═O)R¹¹, —S(O)ₙR¹¹, —C(═O)OR¹², —C₃-C₈ cycloalkyl, —C₄-C₈ cycloalkenyl, -(C₅-C₁₁)bi- or tricycloalkyl, -(C₇-C₁₁)bi- or tricycloalkenyl, -(3-8 membered) heterocycloalkyl, -(C₆-C₁₄)aryl, -(5-14 membered) heteroaryl, -(C₆-C₁₄) aryloxy, and -(5-14 membered) heteroaryloxy, wherein said alkyl, alkoxy, cycloalkyl, cycloalkenyl, bi- or tricycloalkyl, bi- or tricycloalkenyl, heterocycloalkyl, aryl, heteroaryl, aryloxy, and heteroaryloxy are each independently optionally substituted with from one to three substituents R^{1b};
R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, -(5-15 membered) heteroaryl, and —C₁-C₆ alkyl independently optionally containing from one to three double or triple bonds and independently substituted with from one to six atoms independently selected from F, Cl, Br, and I;
R² is selected from —H, —C₁-C₄ alkyl optionally containing one or two double or triple bonds, —C(═O)(C₁-C₄ alkyl), —C₆-C₁₀ aryl, —SO₂—(C₆-C₁₀ aryl), and —SO₂—CH₂—(C₆-C₁₀ aryl), and R² is optionally substituted with from one to three substituents R^{1b};
R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);
R⁴ is H, D, F, or C₁-C₄ alkyl;
or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;
R⁵ is selected from —H, —C₁-C₆ alkyl optionally substituted with from one to three R^{1a}, and —C₆-C₁₀ aryl optionally substituted with from one to three R^{1a};
or R⁵ and R¹ may together optionally form a five to fourteen membered heteroaryl ring or a five to eight membered heterocycloalkyl ring, wherein said heteroaryl ring optionally contains one or two further heteroatoms independently selected from N, O, and S, and said heterocycloalkyl ring optionally contains one or two further heteroatoms independently selected from N—R⁹, O, and S(O)_{zero-2}, and wherein said heterocycloalkyl ring optionally contains from one to three double bonds, and wherein said heteroaryl or heterocycloalkyl ring is optionally substituted from one to three substituents R^{1b};
R⁶ is selected from —H, —C₁-C₂₀ alkyl, —Cl, —F, —Br, —I, —CN, —CF₃, —C(═O)R¹¹, —C(═O)OR¹², —S(O)ₙNR⁹R¹⁰, —S(O)ₙR¹¹, —C(═NR⁹)R¹⁵, -(C₃-C₁₂) cycloalkyl, -(C₄-C₁₂) cycloalkenyl, and —C₆-C₁₀ aryl, wherein said alkyl, alkylene, cycloalkyl, cycloalkenyl, and aryl of R⁶ are each optionally substituted with from one to three substituents R^{1b};
R⁷ is selected from H, —Cl, —F, —Br, —I, —CN, —NO₂, —NR¹⁴R¹⁵, —CF₃, —C(═O)NR¹⁴R¹⁵, —C(═O)R¹³, —S(O)ₙR¹³, —C(═O)OR¹³, —C(═NR⁹)R¹⁵, —S(O)ₙNR¹⁴R¹⁵, —C₁-C₂₀ alkyl, —C₁-C₂₀ alkoxy, -(C_{zero}-C₄ alkylene)-(C₃-C₁₂ cycloalkyl), -(C_{zero}-C₄ alkylene)-((C₄-C₁₂)cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-((3-12 membered) heterocycloalkyl), -(C_{zero}-C₄ alkylene)-((7-20 membered) heterobi- or heterotricycloalkyl), -(C_{zero}-C₄ alkylene)-((C₆-C₁₄)aryl), and -(C_{zero}-C₄ alkylene)-((5-15 membered) heteroaryl); wherein R⁷ is optionally substituted with from one to three substituents independently selected from R^{1a}, -(CH₂)₁₋₁₀NR⁹R¹⁰, —C₃-C₁₂ cycloalkyl, -((4-12 membered) heterocycloalkyl), -(C₆-C₁₄) aryl, -((5-15 membered) heteroaryl), -(4-12 membered) heterocycloalkoxy), -(C₆-C₁₂) aryloxy and -((5-12 membered) heteroaryloxy); said cycloalkyl, cycloalkenyl, bi- or tricycloalkyl, bi- or tricycloalkenyl, heterocycloalkyl, aryl, and heteroaryl of R⁷ are each optionally and independently substituted with from one to six F; said alkyl, alkoxy, and alkylene of R⁷ each optionally contains from one to five double or triple bonds; and each hydrogen atom of said alkyl, alkoxy, and alkylene of R⁷ is independently optionally replaced with a fluorine;
or R⁶ and R⁷ may together optionally form a -(C₆-C₁₀) aryl ring, -(C₆-C₈) cycloalkyl or cycloalkenyl ring, a five to eight membered heterocycloalkyl or heterocycloalkenyl ring, a -(C₁₀-C₁₄) membered bicycloalkyl or bicycloalkenyl ring, or a ten to fourteen membered heterobicycloalkyl or heterobicycloalkenyl ring fused to the thiazole ring of Formula I, wherein from one to three members of said heterocycloalkyl and heterocycloalkenyl rings, and from one to five members of said heterobicycloalkyl and heterobicycloalkenyl rings are selected independently from N—R⁹, O and S(O)_{zero-2}, and wherein said aryl, cycloalkyl, cycloalkenyl, heterocycloalkyl, heterocycloalkenyl, bicycloalkyl, bicycloalkenyl, heterobicycloalkyl, and heterobicycloalkenyl rings optionally are substituted with from one to three R^{1b};
R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇-C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
or NR⁹R¹⁰ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};
R¹³ is selected from H, —C₁-C₆ alkyl optionally containing from one to three double or triple bonds and wherein each hydrogen is independently optionally replaced with a fluorine, -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), and -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-((3-12 membered) heterocycloalkyl), -(C_{zero}-C₄ alkylene)-((7-20 membered) heterobi- or heterotricycloalkyl), and -(C_{zero}-C₄ alkylene)-((5-14 membered) heteroaryl), and R¹³ is optionally substituted with from one to three substituents R^{1b};
R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;
or NR¹⁴R¹⁵ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I; and
n is in each instance an integer independently selected from zero, 1, 2, and 3.

2. A compound according to claim 1, wherein A is —C(═O)Z- or —C(═O)C(═O)—.

3. A compound according to claim 2, wherein Z is —CH₂— or —CH(OH)—.

4. A compound according to claim 3, wherein R³ is allyl, methyl, ethyl, n-propyl, n-butyl, i-butyl, s-butyl, or —CH₂CH₂SCH₃.

5. A compound according to claim 1, wherein R³ is allyl, methyl, ethyl, n-propyl, n-butyl, i-butyl, s-butyl, or —CH₂CH₂SCH₃.

6. A compound according to claim 1, wherein Z is —CH(NH₂)—.

7. A compound according to claim 2, wherein Z is —CH(NH₂)—.

8. A compound according to claim 1, wherein R⁶ is selected from hydrogen, methyl, ethyl, —F, —Cl, —Br, and —CF₃.

9. A compound according to claim 3, wherein R⁶ is selected from hydrogen, methyl, ethyl, —F, —Cl, —Br, and —CF₃.

10. A compound according to claim 1, wherein R¹ is —C₂-C₁₂ alkyl, C₃-C₈ cycloalkyl, (C₅-C₈)cycloalkenyl, -(C₅-C₁₁)bi- or tricycloalkyl, -(C₇-C₁₁)bi- or tricycloalkenyl, (3-8 membered) heterocycloalkyl), -(C₆-C₁₀)aryl, -(5-10 membered) heteroaryl, or C₁-C₄ alkyl substituted with R^{1a} wherein R^{1a} is -(C₆-C₁₀)aryl or -(5-10 membered) heteroaryl.

11. A compound according to claim 3, wherein R¹ is —C₂-C₁₂ alkyl, C₃-C₈ cycloalkyl, (C₅-C₈)cycloalkenyl, -(C₅-C₁₁)bi- or tricycloalkyl, -(C₇-C₁₁)bi- or tricycloalkenyl, (3-8 membered) heterocycloalkyl), -(C₆-C₁₀)aryl, -(5-10 membered) heteroaryl, or C₁-C₄ alkyl substituted with R^{1a} wherein R^{1a} is -(C₆-C₁₀)aryl or -(5-10 membered) heteroaryl.

12. A compound according to claim 1, wherein R¹ is straight-chain C₂-C₁₀ alkyl or branched C₃-C₁₀ alkyl.

13. A compound according to claim 3, wherein R¹ is straight-chain C₂-C₁₀ alkyl or branched C₃-C₁₀ alkyl.

14. A compound according to claim 1, wherein R¹ is C₃-C₁₀ alkyl comprising a tertiary carbon or C₄-C₁₀ alkyl comprising a quaternary carbon.

15. A compound according to claim 3, wherein R¹ is C₃-C₁₀ alkyl comprising a tertiary carbon or C₄-C₁₀ alkyl comprising a quaternary carbon.

16. A compound according to claim 1, wherein R¹ is selected from phenyl, thienyl, and pyridyl, optionally and independently substituted with one or two substituents R^{1b}.

17. A compound according to claim 3, wherein R¹ is selected from phenyl, thienyl, and pyridyl, optionally and independently substituted with one or two substituents R^{1b}.

18. A compound according to claim 1, wherein R⁷ is selected from —H, —C₁-C₁₂ alkyl optionally containing from one to five double bonds and wherein each hydrogen is independently optionally replaced with a fluorine, —C₁-C₂₀ alkoxy optionally containing from one to five double bonds and wherein each hydrogen is independently optionally replaced with a fluorine, —F, —Cl, —Br, —I, —CN, —NO₂, -(C₃-C₁₂) cycloalkyl optionally substituted with from one to six fluorine, -((3-12 membered) heterocycloalkyl) optionally substituted with from one to six fluorine, -(C₆-C₁₄) aryl, -((5-15 membered) heteroaryl), —CHO, —C(═O)(C₁-C₁₅ alkyl), —C(═O)((5-12 membered)heterocycloalkyl), —C(═O)(C₆-C₁₄ aryl), —C(═O)((5-15 membered) heteroaryl), —C(═O)(C₅-C₁₂ cycloalkyl), —C(═O)O(C₁-C₈ alkyl), —C(═O)N(C₁-C₁₀ alkyl)(C₁-C₁₀ alkyl), —C(═O)N(C₁-C₁₀ alkyl)(C₆-C₁₀ aryl), —C(═O)NH(C₆-C₁₀ aryl), —C(═O)N(C₁-C₁₀ alkyl)((5-10 membered) heteroaryl), —C(═O)NH((5-10 membered) heteroaryl), —C(═O)N(C₁-C₁₀ alkyl)((5-10 membered) heterocycloalkyl), —C(═O)NH((5-10 membered) heterocycloalkyl), —C(═O)N(C₁-C₁₀ alkyl)(C₅-C₁₀ cycloalkyl), —C(═O)NH(C₅-C₁₀ cycloalkyl), —S(O)ₙ(C₁-C₁₅ alkyl), —S(O)ₙ(C₅-C₁₂ cycloalkyl), —S(O)ₙ(C₆-C₁₅ aryl), —S(O)ₙ((5-10 membered) heteroaryl), wherein said alkyl, cycloalkyl, heterocycloalkyl, aryl and heteroaryl are each optionally independently substituted with from one to three substituents independently selected from —F, —Cl, —Br, —I, —OH, —C₁-C₆ alkoxy independently optionally containing from one to three double or triple bonds, —NR⁹R¹⁰, -(CH₂)₁₋₁₀NR⁹R¹⁰, —C(═O)R¹¹, —S(O)ₙR¹¹, —C(═O)OR¹¹, —C(═O)NR⁹R¹⁰, —S(O)ₙNR⁹R¹⁰ -(C₃-C₁₂) cycloalkyl, -((4-12 membered) heterocycloalkyl), -(C₆-C₁₅) aryl, -((5-15 membered) heteroaryl), -((4-12 membered) heterocycloalkoxy), -(C₆-C₁₂) aryloxy and -((6-12 membered) heteroaryloxy).

19. A compound according to claim 3, wherein R⁷ is selected from —H, —C₁-C₁₂ alkyl optionally containing from one to five double bonds and wherein each hydrogen is independently optionally replaced with a fluorine, —C₁-C₂₀ alkoxy optionally containing from one to five double bonds and wherein each hydrogen is independently optionally replaced with a fluorine, —F, —Cl, —Br, —I, —CN, —NO₂, -(C₃-C₁₂) cycloalkyl optionally substituted with from one to six fluorine, -((3-12 membered) heterocycloalkyl) optionally substituted with from one to six fluorine, -(C₆-C₁₄) aryl, -((5-15 membered) heteroaryl), —CHO, —C(═O)(C₁-C₁₅ alkyl), —C(═O)((5-12 membered)heterocycloalkyl), —C(═O)(C₆-C₁₄ aryl), —C(═O)((5-15 membered) heteroaryl), —C(═O)(C₅-C₁₂ cycloalkyl), —C(═O)O(C₁-C₈ alkyl), —C(═O)N(C₁-C₁₀ alkyl)(C₁-C₁₀ alkyl), —C(═O)N(C₁-C₁₀ alkyl)(C₆-C₁₀ aryl), —C(═O)NH(C₆-C₁₀ aryl), —C(═O)N(C₁-C₁₀ alkyl)((5-10 membered) heteroaryl), —C(═O)NH((5-10 membered) heteroaryl), —C(═O)N(C₁-C₁₀ alkyl)((5-10 membered) heterocycloalkyl), —C(═O)NH((5-10 membered) heterocycloalkyl), —C(═O)N(C₁-C₁₀ alkyl)(C₅-C₁₀ cycloalkyl), —C(═O)NH(C₅-C₁₀ cycloalkyl), —S(O)ₙ(C₁-C₁₅ alkyl), —S(O)ₙ(C₅-C₁₂ cycloalkyl), —S(O)ₙ(C₆-C₁₅ aryl), —S(O)ₙ((5-10 membered) heteroaryl), wherein said alkyl, cycloalkyl, heterocycloalkyl, aryl and heteroaryl are each optionally independently substituted with from one to three substituents independently selected from —F, —Cl, —Br, —I, —OH, —C₁-C₆ alkoxy independently optionally containing from one to three double or triple bonds, —NR⁹R¹⁰, -(CH₂)₁₋₁₀NR⁹R¹⁰, —C(═O)R¹¹, —S(O)ₙR¹¹, —C(═O)OR¹¹, —C(═O)NR⁹R¹⁰, —S(O)ₙNR⁹R¹⁰, -(C₃-C₁₂) cycloalkyl, -((4-12 membered) heterocycloalkyl), -(C₆-C₁₅) aryl, -((5-15 membered) heteroaryl), -((4-12 membered) heterocycloalkoxy), -(C₆-C₁₂) aryloxy and -((6-12 membered) heteroaryloxy).

20. A compound according to claim 18, wherein R⁷ is selected from —C₁-C₁₂ alkyl optionally comprising from one to five double bonds and wherein each hydrogen is independently optionally replaced with a fluorine, -(C₃-C₁₂) cycloalkyl optionally substituted with from one to six fluorine, and -((3-12 membered) heterocycloalkyl) optionally substituted with from one to six fluorine, wherein said alkyl, cycloalkyl and heterocycloalkyl are each optionally independently substituted with from one to three substitutents independently selected from —OH, —C₁-C₆ alkoxy independently optionally containing from one to three double or triple bonds, —NR⁹R¹⁰, -(CH₂)₁₋₆NR⁹R¹⁰, —C(═O)R¹¹, —C(═O)OR¹¹, —C(═O)NR⁹R¹⁰, —S(O)ₙNR⁹R¹⁰, -(C₆-C₁₅) aryl, -((5-15 membered) heteroaryl), -((4-12 membered) heterocycloalkoxy), -(C₆-C₁₂) aryloxy and -((6-12 membered) heteroaryloxy).

21. A compound according to claim 19, wherein R⁷ is selected from —C₁-C₁₂ alkyl optionally comprising from one to five double bonds and wherein each hydrogen is independently optionally replaced with a fluorine, -(C₃-C₁₂) cycloalkyl optionally substituted with from one to six fluorine, and -((3-12 membered) heterocycloalkyl) optionally substituted with from one to six fluorine, wherein said alkyl, cycloalkyl and heterocycloalkyl are each optionally independently substituted with from one to three substitutents independently selected from —OH, —C₁-C₆ alkoxy independently optionally containing from one to three double or triple bonds, —NR⁹R¹⁰, -(CH₂)₁₋₆NR⁹R¹⁰, —C(═O)R¹¹, —C(═O)OR¹¹, —C(═O)NR⁹R¹⁰, —S(O)ₙNR⁹R¹⁰, -(C₆-C₁₅) aryl, -((5-15 membered) heteroaryl), -((4-12 membered) heterocycloalkoxy), -(C₆-C₁₂) aryloxy and -((6-12 membered) heteroaryloxy).

22. A compound according to claim 1 selected from the group:
2-[2-(3,5-difluoro-phenyl)-acetylamino]-N-(4-phenyl-thiazol-2-yl)-propionamide;
2-{2-[2-(3,5-difluoro-phenyl)-acetylamino]-propionylamino}-4-phenyl-thiazole-5-carboxylic acid ethyl ester;
(2-{2-[2-(3,5-difluoro-phenyl)-acetylamino]-pentanoylamino}-thiazol-4-yl)-acetic acid ethyl ester;
2-[2-(3,5-difluoro-phenyl)-acetylamino]-pentanoic acid [5-(4-nitro-benzenesulfonyl)-thiazol-2-yl]-amide;
2-[2-(3,5-difluoro-phenyl)-acetylamino]-pentanoic acid [5-(4-hydroxyamino-benzenesulfonyl)-thiazol-2-yl]-amide;
2-[2-(3,5-difluoro-phenyl)-acetylamino]-pentanoic acid [5-(4-amino-benzenesulfonyl)-thiazol-2-yl]-amide;
N-[5-(5-bromo-thiophen-2-yl)-thiazol-2-yl]-2-[2-(3,5-difluoro-phenyl)-acetylamino]-butyramide;
2-[2-(3,5-difluoro-phenyl)-acetylamino]-pentanoic acid [5-(4-benzylamino-benzenesulfonyl)-thiazol-2-yl]-amide;
2-[2-(3,5-difluoro-phenyl)-acetylamino]-pentanoic acid benzothiazol-2-ylamide;
2-[2-(3,5-difluoro-phenyl)-acetylamino]-pentanoic acid (5-methyl-thiazol-2-yl)-amide;
2-[2-(3,5-difluoro-phenyl)-acetylamino]-pentanoic acid (4,5-dimethyl-thiazol-2-yl)-amide;
2-[2-(3,5-difluoro-phenyl)-acetylamino]-pentanoic acid (5-nitro-thiazol-2-yl)-amide;
2-[2-(3,5-difluoro-phenyl)-acetylamino]-pentanoic acid thiazol-2-ylamide;
2-[2-(3,5-difluoro-phenyl)-acetylamino]-pentanoic acid (5,6-dihydro-4H-cyclopentathiazol-2-yl)-amide;
2-[2-(3,5-difluoro-phenyl)-acetylamino]-pentanoic acid (5-chloro-thiazol-2-yl)-amide;
2-[2-(3,5-difluoro-phenyl)-acetylamino]-pentanoic acid (4-methyl-thiazol-2-yl)-amide;
(2-{2-[2-(3,5-difluoro-phenyl)-acetylamino]-pentanoylamino}-thiazol-4-yl)-acetic acid;
2-[2-(3,5-difluoro-phenyl)-acetylamino]-pentanoic acid (5-amino-thiazol-2-yl)-amide;
2-[2-(3,5-difluoro-phenyl)-acetylamino]-pentanoic acid [5-(4-chloro-benzenesulfonyl)-thiazol-2-yl]-amide;
2-[2-(3,5-difluoro-phenyl)-acetylamino]-N-[5-(5-methoxy-1,5-dimethyl-hexyl)-thiazol-2-yl]-butyramide;
2-[2-(3,5-difluoro-phenyl)-acetylamino]-N-(5,6,7,8-tetrahydro-4H-cycloheptathiazol-2-yl)-butyramide;
N-(4-cyclopentyl-thiazol-2-yl)-2-[2-(3,5-difluoro-phenyl)-acetylamino]-butyramide;
2-[2-(3,5-difluoro-phenyl)-acetylamino]-N-(6-methyl-4,5,6,7-tetrahydro-benzothiazol-2-yl)-butyramide;
2-[2-(3,5-difluoro-phenyl)-acetylamino]-N-(5-methylsulfanyl-thiazol-2-yl)-butyramide;
2-[2-(3,5-difluoro-phenyl)-acetylamino]-N-(5-isopropyl-thiazol-2-yl)-butyramide;
2-[2-(3,5-difluoro-phenyl)-acetylamino]-pentanoic acid {4-[(butyl-ethyl-carbamoyl)-methyl]-thiazol-2-yl}-amide;
2-[2-(3,5-difluoro-phenyl)-acetylamino]-pentanoic acid [4-(benzylcarbamoyl-methyl)-thiazol-2-yl]-amide;
2-[2-(3,5-difluoro-phenyl)-acetylamino]-pentanoic acid (5-bromo-thiazol-2-yl)-amide;
2-[2-(3,5-difluoro-phenyl)-acetylamino]-N-(4-phenyl-thiazol-2-yl)-butyramide;
2-[2-(3,5-difluoro-phenyl)-acetylamino]-N-(4,5-diphenyl-thiazol-2-yl)-butyramide;
N-(5-acetyl-thiazol-2-yl)-2-[2-(3,5-difluoro-phenyl)-acetylamino]-butyramide;
2-[2-(3,5-difluoro-phenyl)-acetylamino]-pentanoic acid (4-ethylcarbamoylmethyl-thiazol-2-yl)-amide;
N-(5-sec-butyl-thiazol-2-yl)-2-[2-(3,5-difluoro-phenyl)-acetylamino]-butyramide;
2-[2-(3,5-difluoro-phenyl)-acetylamino]-N-(6-methyl-benzothiazol-2-yl)-butyramide;
2-[2-(3,5-difluoro-phenyl)-acetylamino]-N-(6-methoxy-benzothiazol-2-yl)-butyramide;
N-(6-chloro-benzothiazol-2-yl)-2-[2-(3,5-difluoro-phenyl)-acetylamino]-butyramide;
N-(4-chloro-benzothiazol-2-yl)-2-[2-(3,5-difluoro-phenyl)-acetylamino]-butyramide;
2-[2-(3,5-difluoro-phenyl)-acetylamino]-pentanoic acid {4-[(cyclopropylmethyl-carbamoyl)-methyl]-thiazol-2-yl}-amide;
3,7-dimethyl-oct-6-enoic acid [1-(5-methyl-thiazol-2-ylcarbamoyl)-butyl]-amide;
2-(2-cyclohexyl-2-hydroxy-acetylamino)-pentanoic acid (5-methyl-thiazol-2-yl)-amide;
2-[2-(3,5-difluoro-phenyl)-acetylamino]-N-(4,5,6,7-tetrahydro-benzothiazol-2-yl)-butyramide;
2-(2-{2-[2-(3,5-difluoro-phenyl)-acetylamino]-butyrylamino}-thiazol-4-yl)-2-methyl-propionic acid ethyl ester;
2-[2-(3,5-difluoro-phenyl)-acetylamino]-N-[6-(piperidine-1-sulfonyl)-benzothiazol-2-yl]-butyramide;
2-[2-(3,5-difluoro-phenyl)-2-hydroxy-acetylamino]-pentanoic acid (5-methyl-thiazol-2-yl)-amide;
2-[2-(3,5-difluoro-phenyl)-acetylamino]-N-[5-(4-fluoro-phenyl)-thiazol-2-yl]-butyramide;
(2-{2-[2-(3,5-difluoro-phenyl)-acetylamino]-butyrylamino}-thiazol-4-yl)-methoxyimino-acetic acid ethyl ester;
2-[2-(5-bromo-pyridin-3-yl)-acetylamino]-pentanoic acid (5-methyl-thiazol-2-yl)-amide;
2-[2-(3-phenoxy-phenyl)-acetylamino]-pentanoic acid (5-butyl-thiazol-2-yl)-amide;
2-(2-hydroxy-3-methyl-butyrylamino)-pentanoic acid (5-isopropyl-thiazol-2-yl)-amide;
2-[2-(3,5-difluoro-phenyl)-acetylamino]-pentanoic acid (5-isopropyl-thiazol-2-yl)-amide;
4-methyl-2-{2-[2-(3-phenoxy-phenyl)-acetylamino]-pentanoylamino}-thiazole-5-carboxylic acid dimethylamide;
2-[2-(5-bromo-pyridin-3-yl)-acetylamino]-pentanoic acid (5-isopropyl-thiazol-2-yl)-amide;
3,7-dimethyl-oct-6-enoic acid [1-(5-isopropyl-thiazol-2-ylcarbamoyl)-butyl]-amide;
2-(2-hydroxy-3-methyl-butyrylamino)-pentanoic acid (5-isopropyl-thiazol-2-yl)-amide;
2-hydroxy-N-[2-(2-hydroxy-3-methyl-butyrylamino)-pentanoyl]-N-(5-isopropyl-thiazol-2-yl)-3-methyl-butyramide;
3,7-dimethyl-oct-6-enoic acid [1-(5-isopropyl-thiazol-2-ylcarbamoyl)-butyl]-amide;
2-{2-[2-(3,5-difluoro-phenyl)-acetylamino]-pentanoylamino}-4-ethoxymethyl-thiazole-5-carboxylic acid ethyl ester;
2-{2-[2-(3,5-difluoro-phenyl)-acetylamino]-pentanoylamino}-thiazole-5-carboxylic acid amide;
2-[2-(3,5-difluoro-phenyl)-acetylamino]-pentanoic acid {5-[(4-hydroxy-4-phenyl-piperidin-1-yl)-acetyl]-thiazol-2-yl}-amide;
2-[2-(3,5-difluoro-phenyl)-acetylamino]-pentanoic acid [5-(methyl-phenyl-amino)-thiazol-2-yl]-amide;
2-{2-[2-(3,5-difluoro-phenyl)-acetylamino]-pentanoylamino}-4-methyl-thiazole-5-carboxylic acid (4-chloro-phenyl)-amide;
2-{2-[2-(3,5-difluoro-phenyl)-acetylamino]-pentanoylamino}-thiazole-5-carboxylic acid methyl ester;
2-[2-(3,5-difluoro-phenyl)-acetylamino]-pentanoic acid (5-acetyl-thiazol-2-yl)-amide;
(2-{2-[2-(3,5-Difluoro-phenyl)-acetylamino]-pentanoylamino}-thiazol-4-yl)-acetic acid ethyl ester;
(2-{2-[2-(3,5-Difluoro-phenyl)-acetylamino]-butyrylamino}-thiazol-4-yl)-methoxyimino-acetic acid ethyl ester;
2-{2-[2-(3,5-Difluoro-phenyl)-acetylamino]-pentanoylamino}-thiazole-5-carboxylic acid methyl ester;
2-[2-(3,5-Difluoro-phenyl)-acetylamino]-pentanoic acid (5-acetyl-thiazol-2-yl)-amide;
2-[2-(3,5-Difluoro-phenyl)-2-hydroxy-acetylamino]-pentanoic acid [5-(5-methoxy-1,5-dimethyl-hexyl)-thiazol-2-yl]-amide;
2-(2-Hydroxy-3,3-dimethyl-butyrylamino)-pentanoic acid (5-isopropyl-thiazol-2-yl)-amide;
2-[2-(3,5-Difluoro-phenyl)-2-hydroxy-acetylamino]-pentanoic acid (5-methyl-thiazol-2-yl)-amide;
2-[2-(3,5-Difluoro-phenyl)-2-hydroxy-acetylamino]-pentanoic acid (5-methyl-thiazol-2-yl)-amide;
Hydroxy-phenyl-acetic acid [1-(5-isopropyl-thiazol-2-ylcarbamoyl)-butylcarbamoyl]-phenyl-methyl ester;
2-(2-Hydroxy-2-phenyl-acetylamino)-pentanoic acid (5-isopropyl-thiazol-2-yl)-amide;
2-(2-Hydroxy-3-methyl-butyrylamino)-pentanoic acid (5-methyl-thiazol-2-yl)-amide;
2-(2-Hydroxy-2-phenyl-acetylamino)-pentanoic acid (4,5-dimethyl-thiazol-2-yl)-amide;
2-[2-(3,5-Difluoro-phenyl)-acetylamino]-N-[5-(1,5-dimethyl-hex-4-enyl)-thiazol-2-yl]-butyramide;
2-[2-(3,5-Difluoro-phenyl)-acetylamino]-hexanoic acid (5-isopropyl-thiazol-2-yl)-amide;
2-[2-(3,5-Difluoro-phenyl)-acetylamino]-N-[5-(5-hydroxy-1,5-dimethyl-hexyl)-thiazol-2-yl]-propionamide;
2-[2-(3,5-Difluoro-phenyl)-acetylamino]-N-[5-(5-methoxy-1,5-dimethyl-hexyl)-thiazol-2-yl]-propionamide;
2-[2-(3,5-Difluoro-phenyl)-2-hydroxy-acetylamino]-N-[5-(5-hydroxy-1,5-dimethyl-hexyl)-thiazol-2-yl]-propionamide;
2-[2-(3,5-Difluoro-phenyl)-2-hydroxy-acetylamino]-N-[5-(5-methoxy-1,5-dimethyl-hexyl)-thiazol-2-yl]-propionamide;
2-Hydroxy-N-{1-[5-(5-hydroxy-1,5-dimethyl-hexyl)-thiazol-2-ylcarbamoyl]-ethyl}-3-methyl-butyramide;
2-Hydroxy-N-{1-[5-(5-methoxy-1,5-dimethyl-hexyl)-thiazol-2-ylcarbamoyl]-ethyl}-3-methyl-butyramide;
2-Hydroxy-N-{1-[5-(5-hydroxy-1,5-dimethyl-hexyl)-thiazol-2-ylcarbamoyl]-ethyl}-3,3-dimethyl-butyramide;
2-Hydroxy-N-{1-[5-(5-methoxy-1,5-dimethyl-hexyl)-thiazol-2-ylcarbamoyl]-ethyl}-3,3-dimethyl-butyramide;
N-[5-(5-Hydroxy-1,5-dimethyl-hexyl)-thiazol-2-yl]-2-(2-hydroxy-2-phenyl-acetylamino)-propionamide;
2-(2-Hydroxy-2-phenyl-acetylamino)-N-[5-(5-methoxy-1,5-dimethyl-hexyl)-thiazol-2-yl]-propionamide;
N-[5-(5-Hydroxy-1,5-dimethyl-hexyl)-thiazol-2-yl]-2-(2-oxo-2-thiophen-2-yl-acetylamino)-propionamide;
N-[5-(5-Methoxy-1,5-dimethyl-hexyl)-thiazol-2-yl]-2-(2-oxo-2-thiophen-2-yl-acetylamino)-propionamide;
2-[2-(5-Bromo-pyridin-3-yl)-acetylamino]-N-[5-(5-hydroxy-1,5-dimethyl-hexyl)-thiazol-2-yl]-propionamide;
2-[2-(5-Bromo-pyridin-3-yl)-acetylamino]-N-[5-(5-methoxy-1,5-dimethyl-hexyl)-thiazol-2-yl]-propionamide;
2-[2-(3,5-Difluoro-phenyl)-acetylamino]-pentanoic acid [5-(1,3,3-trimethyl-butyl)-thiazol-2-yl]-amide;
2-(2-Hydroxy-3,3-dimethyl-butyrylamino)-pentanoic acid [5-(5-methoxy-1,5-dimethyl-hexyl)-thiazol-2-yl]-amide;
2-(2-Hydroxy-3-methyl-butyrylamino)-pentanoic acid [5-(5-methoxy-1,5-dimethyl-hexyl)-thiazol-2-yl]-amide;
2-[2-(3,5-Difluoro-phenyl)-3-hydroxy-3-methyl-butyrylamino]-pentanoic acid thiazol-2-ylamide;
2-[2-(3,5-Difluoro-phenyl)-acetylamino]-pentanoic acid [5-(1-hydroxy-1-methyl-ethyl)-thiazol-2-yl]-amide;
2-[2-(2-Hydroxy-3,3-dimethyl-butyrylamino)-butyrylamino]-4-trifluoromethyl-thiazole-5-carboxylic acid ethyl ester;
2-[2-(3,5-Difluoro-phenyl)-acetylamino]-pentanoic acid benzyl-thiazol-2-yl-amide;
2-(2-Oxo-2-thiophen-2-yl-acetylamino)-pentanoic acid [5-(5-methoxy-1,5-dimethyl-hexyl)-thiazol-2-yl]-amide;
2-[2-(3,5-Difluoro-phenyl)-acetylamino]-N-[5-(5-methoxy-1,5-dimethyl-hexyl)-thiazol-2-yl]-butyramide;
2-(2-Oxo-2-thiophen-2-yl-acetylamino)-pentanoic acid [5-(5-hydroxy-1,5-dimethyl-hexyl)-thiazol-2-yl]-amide;
2-(2-Oxo-2-thiophen-2-yl-acetylamino)-pentanoic acid [5-(1,5-dimethyl-hex-4-enyl)-thiazol-2-yl]-amide;
2-(3,3-Dimethyl-2-oxo-butyrylamino)-pentanoic acid (5-isopropyl-thiazol-2-yl)-amide;
2-[2-(3,5-Difluoro-phenyl)-acetylamino]-pentanoic acid [5-(1-ethyl-1-hydroxy-propyl)-thiazol-2-yl]-amide;
2-(2-Hydroxy-2-phenyl-acetylamino)-pentanoic acid [5-(5-hydroxy-1,5-dimethyl-hexyl)-thiazol-2-yl]-amide;
2-[2-(3,5-Difluoro-phenyl)-acetylamino]-N-[5-(5-methoxy-1,5-dimethyl-hexyl)-thiazol-2-yl]-butyramide;
2-(2-Hydroxy-2-phenyl-acetylamino)-pentanoic acid [5-(5-methoxy-1,5-dimethyl-hexyl)-thiazol-2-yl]-amide;
2-Hydroxy-N-[1-(5-isopropyl-thiazol-2-ylcarbamoyl)-ethyl]-3,3-dimethyl-butyramide;
2-(2-Hydroxy-2-phenyl-acetylamino)-N-(5-isopropyl-thiazol-2-yl)-butyramide;
2-(2-Hydroxy-2-phenyl-acetylamino)-N-(5-isopropyl-thiazol-2-yl)-propionamide;
2-Hydroxy-N-[1-(5-isopropyl-thiazol-2-ylcarbamoyl)-ethyl]-3-methyl-butyramide;
2-Hydroxy-N-[1-(5-isopropyl-thiazol-2-ylcarbamoyl)-propyl]-3-methyl-butyramide;
2-Hydroxy-3,3-dimethyl-butyric acid 1-[1-(5-isopropyl-thiazol-2-ylcarbamoyl)-ethylcarbamoyl]-2,2-dimethyl-propyl ester;
Hydroxy-phenyl-acetic acid [1-(5-isopropyl-thiazol-2-ylcarbamoyl)-propylcarbamoyl]-phenyl-methyl ester;
2-Hydroxy-3-methyl-butyric acid 1-[1-(5-isopropyl-thiazol-2-ylcarbamoyl)-propylcarbamoyl]-2-methyl-propyl ester;
2-Hydroxy-3-methyl-butyric acid 1-{1-[1-(5-isopropyl-thiazol-2-ylcarbamoyl)-propylcarbamoyl]-2-methyl-propoxycarbonyl}-2-methyl-propyl ester;
2-[2-(5-Bromo-pyridin-3-yl)-2-hydroxy-acetylamino]-N-[5-(5-methoxy-1,5-dimethyl-hexyl)-thiazol-2-yl]-butyramide;
2-[2-(5-Bromo-pyridin-3-yl)-acetylamino]-N-(5-isopropyl-thiazol-2-yl)-butyramide;
Hydroxy-phenyl-acetic acid [1-(5-isopropyl-thiazol-2-ylcarbamoyl)-ethylcarbamoyl]-phenyl-methyl ester;
2-Hydroxy-3-methyl-butyric acid 1-[1-(5-isopropyl-thiazol-2-ylcarbamoyl)-ethylcarbamoyl]-2-methyl-propyl ester;
2-Hydroxy-N-[1-(5-isopropyl-thiazol-2-ylcarbamoyl)-propyl]-3,3-dimethyl-butyramide;
2-(2-Hydroxy-3,3-dimethyl-butyrylamino)-pentanoic acid (5-isopropenyl-thiazol-2-yl)-amide;
2-(2-Hydroxy-3,3-dimethyl-butyrylamino)-pentanoic acid [5-(1-hydroxy-1-methyl-ethyl)-thiazol-2-yl]-amide;
2-[2-(5-Bromo-pyridin-3-yl)-acetylamino]-N-(5-isopropyl-thiazol-2-yl)-propionamide;
2-(3,5-Difluoro-phenyl)-3-hydroxy-3-methyl-pentanoic acid [1-(thiazol-2-ylcarbamoyl)-butyl]-amide;
2-(2-Hydroxy-3,3-dimethyl-butyrylamino)-pentanoic acid [5-(1,3,3-trimethyl-butyl)-thiazol-2-yl]-amide;
1-(3,5-Difluoro-phenyl)-cyclopentanecarboxylic acid [1-(5-methyl-thiazol-2-ylcarbamoyl)-butyl]-amide;
2-[2-(3,5-Difluoro-phenyl)-acetylamino]-N-[5-(1,5-dimethyl-hex-4-enyl)-thiazol-2-yl]-propionamide;
2-[2-(3,5-Difluoro-phenyl)-acetylamino]-pentanoic acid [5-(1-butylamino-ethyl)-thiazol-2-yl]-amide;
2-[2-(3,5-Difluoro-phenyl)-acetylamino]-pentanoic acid [5-(1-butylamino-ethyl)-thiazol-2-yl]-amide;
2-[2-(3,5-Difluoro-phenyl)-acetylamino]-pentanoic acid [5-(1-ethyl-propyl)-thiazol-2-yl]-amide;
2-(2-Hydroxy-3,3-dimethyl-butyrylamino)-pentanoic acid [5-(1,5-dimethyl-hex-4-enyl)-thiazol-2-yl]-amide;
2-(2-Amino-3,3-dimethyl-butyrylamino)-pentanoic acid (5-isopropyl-thiazol-2-yl)-amide;
2-(2-Hydroxy-3,3-dimethyl-butyrylamino)-pentanoic acid [5-(5-methoxy-1,5-dimethyl-hexyl)-thiazol-2-yl]-amide;
2-(2-Hydroxy-3,3-dimethyl-butyrylamino)-pentanoic acid [5-(5-methoxy-1,5-dimethyl-hexyl)-thiazol-2-yl]-amide;
2-(2-Hydroxy-3,3-dimethyl-butyrylamino)-pentanoic acid [5-(5-hydroxy-1,5-dimethyl-hexyl)-thiazol-2-yl]-amide;
2-(2-Hydroxy-3,3-dimethyl-butyrylamino)-pentanoic acid [5-(5-hydroxy-1,5-dimethyl-hexyl)-thiazol-2-yl]-amide;
2-(2-Hydroxy-3-methyl-butyrylamino)-pentanoic acid [5-(5-methoxy-1,5-dimethyl-hexyl)-thiazol-2-yl]-amide;
2-(2-Hydroxy-3-methyl-butyrylamino)-pentanoic acid [5-(5-methoxy-1,5-dimethyl-hexyl)-thiazol-2-yl]-amide;
2-(2-Hydroxy-3,3-dimethyl-butyrylamino)-pentanoic acid (5-acetyl-thiazol-2-yl)-amide;
2-(2-Hydroxy-3,3-dimethyl-butyrylamino)-pentanoic acid (5-acetyl-thiazol-2-yl)-amide;
2-[2-(3,5-Difluoro-phenyl)-acetylamino]-pentanoic acid [5-(1-propyl-butyl)-thiazol-2-yl]-amide;
2-[2-(3,5-Difluoro-phenyl)-acetylamino]-pentanoic acid [5-(1-propyl-butyl)-thiazol-2-yl]-amide;
2-[2-(3,5-Difluoro-phenyl)-acetylamino]-pentanoic acid [5-(1-ethyl-3-methyl-butyl)-thiazol-2-yl]-amide;
2-[2-(3,5-Difluoro-phenyl)-acetylamino]-pentanoic acid [5-(1-ethyl-1-hydroxy-propyl)-thiazol-2-yl]-amide;
2-(2-Hydroxy-3,3-dimethyl-butyrylamino)-pentanoic acid [5-(1-ethyl-1-hydroxy-propyl)-thiazol-2-yl]-amide;
2-[2-(3,5-Difluoro-phenyl)-acetylamino]-pentanoic acid [5-(1-hydroxy-ethyl)-thiazol-2-yl]-amide;
2-[2-(3,5-Difluoro-phenyl)-acetylamino]-pentanoic acid (5-acetyl-4-methyl-thiazol-2-yl)-amide;
2-[2-(3,5-Difluoro-phenyl)-acetylamino]-pentanoic acid [5-(1-butylamino-ethyl)-4-methyl-thiazol-2-yl]-amide;
2-[2-(3,5-Difluoro-phenyl)-acetylamino]-pentanoic acid [4-methyl-5-(1-propylamino-ethyl)-thiazol-2-yl]-amide;
2-[2-(3,5-Difluoro-phenyl)-acetylamino]-pentanoic acid [5-(3,3-dimethyl-cyclohexyl)-thiazol-2-yl]-amide;
2-[2-(3,5-Difluoro-phenyl)-acetylamino]-pentanoic acid (5-ethyl-thiazol-2-yl)-amide;
2-[2-(3,5-Difluoro-phenyl)-acetylamino]-pentanoic acid [5-(1-benzyl-4-hydroxy-piperidin-4-yl)-thiazol-2-yl]-amide;
2-[2-(3,5-Difluoro-phenyl)-acetylamino]-pentanoic acid (5-formyl-thiazol-2-yl)-amide;
2-[2-(3,5-Difluoro-phenyl)-acetylamino]-pentanoic acid (5-ethylsulfanyl-thiazol-2-yl)-amide;
2-[2-(3,5-Difluoro-phenyl)-acetylamino]-pentanoic acid (8H-3-thia-1-aza-cyclopenta[a]inden-2-yl)-amide;
2-[2-(3,5-Difluoro-phenyl)-acetylamino]-pentanoic acid [4-phenyl-5-(piperidine-1-carbonyl)-thiazol-2-yl]-amide;
(2-{2-[2-(3,5-Difluoro-phenyl)-acetylamino]-pentanoylamino}-thiazol-5-ylmethylsulfanyl)-acetic acid ethyl ester;
2-[2-(3,5-Difluoro-phenyl)-acetylamino]-pentanoic acid [5-(1-hydroxy-ethyl)-4-methyl-thiazol-2-yl]-amide;
2-[2-(3,5-Difluoro-phenyl)-acetylamino]-pentanoic acid [5-(1-hydroxy-ethyl)-4-methyl-thiazol-2-yl]-amide;
2-[2-(3,5-Difluoro-phenyl)-acetylamino]-pentanoic acid [5-(1-ethyl-propenyl)-thiazol-2-yl]-amide;
2-(2-Hydroxy-2-phenyl-acetylamino)-pentanoic acid [5-(1-ethyl-1-hydroxy-propyl)-thiazol-2-yl]-amide;
2-(2-Hydroxy-2-phenyl-acetylamino)-pentanoic acid [5-(1-ethyl-1-hydroxy-propyl)-thiazol-2-yl]-amide;
2-[2-(3,5-Difluoro-phenyl)-acetylamino]-pentanoic acid {5-[1-(2-methoxy-ethylamino)-ethyl]-4-methyl-thiazol-2-yl}-amide;
2-[2-(3,5-Difluoro-phenyl)-acetylamino]-pentanoic acid [4-methyl-5-(1-pyrrolidin-1-yl-ethyl)-thiazol-2-yl]-amide;
2-(2-Hydroxy-3,3-dimethyl-butyrylamino)-pentanoic acid [5-(1-ethyl-propyl)-thiazol-2-yl]-amide;
2-[2-(3-Phenoxy-phenyl)-acetylamino]-pentanoic acid [5-(1-ethyl-propyl)-thiazol-2-yl]-amide;
2-(2-Hydroxy-3-methyl-butyrylamino)-pentanoic acid [5-(1-ethyl-propyl)-thiazol-2-yl]-amide;
2-[2-(5-Bromo-pyridin-3-yl)-acetylamino]-pentanoic acid [5-(1-ethyl-propyl)-thiazol-2-yl]-amide;
2-[2-(3,5-Difluoro-phenyl)-acetylamino]-pentanoic acid [5-(1,3-dimethyl-but-1-enyl)-thiazol-2-yl]-amide;
2-[2-(3,5-Difluoro-phenyl)-acetylamino]-pentanoic acid [5-(1-isobutyl-vinyl)-thiazol-2-yl]-amide;
2-[2-(3,5-Difluoro-phenyl)-acetylamino]-pentanoic acid {5-[(1-benzyl-piperidin-4-ylamino)-methyl]-thiazol-2-yl}-amide;
2-[2-(3,5-Difluoro-phenyl)-2-hydroxy-acetylamino]-pentanoic acid [5-(1-ethyl-propyl)-thiazol-2-yl]-amide;
2-[2-(3,5-Difluoro-phenyl)-2-hydroxy-acetylamino]-pentanoic acid [5-(1-ethyl-propyl)-thiazol-2-yl]-amide;
2-[2-(3,5-Difluoro-phenyl)-acetylamino]-pentanoic acid [4-methyl-5-(1-methylamino-ethyl)-thiazol-2-yl]-amide;
2-[2-(3,5-Difluoro-phenyl)-acetylamino]-pentanoic acid [5-(1-ethylamino-ethyl)-4-methyl-thiazol-2-yl]-amide;
2-[2-(3,5-Difluoro-phenyl)-acetylamino]-pentanoic acid [5-(1-isopropylamino-ethyl)-4-methyl-thiazol-2-yl]-amide;
2-[2-(3,5-Difluoro-phenyl)-acetylamino]-pentanoic acid {5-[1-(2-hydroxy-ethylamino)-ethyl]-4-methyl-thiazol-2-yl}-amide;
2-[2-(3,5-Difluoro-phenyl)-acetylamino]-pentanoic acid [4-methyl-5-(1-morpholin-4-yl-ethyl)-thiazol-2-yl]-amide;
2-[2-(3,5-Difluoro-phenyl)-acetylamino]-pentanoic acid {4-methyl-5-[1-(4-methyl-piperazin-1-yl)-ethyl]-thiazol-2-yl}-amide;
2-[2-(3,5-Difluoro-phenyl)-acetylamino]-N-[5-(1-ethyl-propyl)-thiazol-2-yl]-propionamide;
N-{1-[5-(1-Ethyl-propyl)-thiazol-2-ylcarbamoyl]-ethyl}-2-hydroxy-3,3-dimethyl-butyramide;
2-(2-Hydroxy-2-phenyl-acetylamino)-pentanoic acid [5-(1-ethyl-propyl)-thiazol-2-yl]-amide;
2-[2-(3,5-Difluoro-phenyl)-acetylamino]-pentanoic acid (5-{[ethyl-(2-hydroxy-ethyl)-amino]-methyl}-thiazol-2-yl)-amide;
2-[2-(3,5-Difluoro-phenyl)-acetylamino]-pentanoic acid {5-[1-(3,3-dimethyl-butylamino)-ethyl]-4-methyl-thiazol-2-yl}-amide;
2-[2-(3,5-Difluoro-phenyl)-acetylamino]-pentanoic acid [5-(1-isobutylamino-ethyl)-4-methyl-thiazol-2-yl]-amide;
2-[2-(3,5-Difluoro-phenyl)-acetylamino]-pentanoic acid {4-methyl-5-[1-(3-methyl-butylamino)-ethyl]-thiazol-2-yl}-amide;
2-[2-(3,5-Difluoro-phenyl)-acetylamino]-pentanoic acid (5-hydroxymethyl-thiazol-2-yl)-amide;
2-[2-(3,5-Difluoro-phenyl)-acetylamino]-pentanoic acid (5-morpholin-4-ylmethyl-thiazol-2-yl)-amide;
2-[2-(3,5-Difluoro-phenyl)-acetylamino]-pentanoic acid {5-[(butyl-ethyl-amino)-methyl]-thiazol-2-yl}-amide;
2-[2-(3,5-Difluoro-phenyl)-acetylamino]-pentanoic acid (5-trimethylsilanyl-thiazol-2-yl)-amide;
2-(2-Hydroxy-3,3-dimethyl-butyrylamino)-pentanoic acid (5-acetyl-4-methyl-thiazol-2-yl)-amide;
2-(2-Hydroxy-3-methyl-butyrylamino)-pentanoic acid (5-acetyl-4-methyl-thiazol-2-yl)-amide;
2-(2-Hydroxy-2-phenyl-acetylamino)-pentanoic acid (5-acetyl-4-methyl-thiazol-2-yl)-amide;
2-(2-Hydroxy-3,3-dimethyl-butyrylamino)-pentanoic acid {5-[1-(5-acetyl-4-methyl-thiazol-2-ylimino)-ethyl]-4-methyl-thiazol-2-yl}-amide;
2-(2-Hydroxy-2-phenyl-acetylamino)-pentanoic acid (5-trifluoroacetyl-thiazol-2-yl)-amide;
2-[2-(3,5-Difluoro-phenyl)-acetylamino]-pentanoic acid {5-[(1-ethyl-propylamino)-methyl]-thiazol-2-yl}-amide;
N-[5-(1-Ethyl-propyl)-thiazol-2-yl]-2-(2-hydroxy-2-phenyl-acetylamino)-propionamide;
N-[5-(1-Ethyl-propyl)-thiazol-2-yl]-2-(2-hydroxy-2-phenyl-acetylamino)-butyramide;
2-[2-(3,5-Difluoro-phenyl)-acetylamino]-pentanoic acid (5-ethylaminomethyl-thiazol-2-yl)-amide;
2-[2-(3,5-Difluoro-phenyl)-acetylamino]-pentanoic acid (5-dimethylaminomethyl-thiazol-2-yl)-amide;
2-[2-(3,5-Difluoro-phenyl)-acetylamino]-pentanoic acid [5-(isopropylamino-methyl)-thiazol-2-yl]-amide;
2-(2-Hydroxy-2-phenyl-acetylamino)-pentanoic acid [5-(2,2,2-trifluoro-1-hydroxy-ethyl)-thiazol-2-yl]-amide;
2-[2-(3,5-Difluoro-phenyl)-acetylamino]-pentanoic acid (5-aminomethyl-thiazol-2-yl)-amide;
2-(2-Hydroxy-3,3-dimethyl-butyrylamino)-pentanoic acid (5-formyl-thiazol-2-yl)-amide;
2-(2-Hydroxy-3,3-dimethyl-butyrylamino)-pentanoic acid [5-(1-propyl-butyl)-thiazol-2-yl]-amide;
2-Hydroxy-3,3-dimethyl-N-{1-[5-(1-propyl-butyl)-thiazol-2-ylcarbamoyl]-propyl}-butyramide;
2-Hydroxy-3,3-dimethyl-N-{1-[5-(1-propyl-butyl)-thiazol-2-ylcarbamoyl]-ethyl}-butyramide;
2-(2-Hydroxy-3,3-dimethyl-butyrylamino)-pentanoic acid (4-methyl-5-vinyl-thiazol-2-yl)-amide;
2-(2-Hydroxy-3,3-dimethyl-butyrylamino)-pentanoic acid {5-[(3-methyl-butylamino)-methyl]-thiazol-2-yl}-amide;
2-(2-Hydroxy-3,3-dimethyl-butyrylamino)-pentanoic acid {5-[(3,3-dimethyl-butylamino)-methyl]-thiazol-2-yl}-amide;
2-(2-Hydroxy-3,3-dimethyl-butyrylamino)-pentanoic acid [5-(isobutylamino-methyl)-thiazol-2-yl]-amide;
2-(2-Hydroxy-3,3-dimethyl-butyrylamino)-pentanoic acid (5-{[methyl-(3-methyl-butyl)-amino]-methyl}-thiazol-2-yl)-amide;
2-(2-Hydroxy-3,3-dimethyl-butyrylamino)-pentanoic acid [5-(1,3,3-trimethyl-butyl)-thiazol-2-yl]-amide;
2-(2-Hydroxy-3-methyl-butyrylamino)-pentanoic acid [5-(1,3,3-trimethyl-butyl)-thiazol-2-yl]-amide;
2-[2-(3,5-Difluoro-phenyl)-acetylamino]-pentanoic acid [4-methyl-5-(1-phenethylamino-ethyl)-thiazol-2-yl]-amide;
2-[2-(3,5-Difluoro-phenyl)-acetylamino]-pentanoic acid [5-(1-benzylamino-ethyl)-4-methyl-thiazol-2-yl]-amide;
2-[2-(3,5-Difluoro-phenyl)-acetylamino]-pentanoic acid (5-acetyl-thiazol-2-yl)-amide;
2-(2-Hydroxy-3-methyl-butyrylamino)-pentanoic acid [5-(5-hydroxy-1,5-dimethyl-hexyl)-thiazol-2-yl]-amide;
2-(2-Hydroxy-3-methyl-butyrylamino)-pentanoic acid [5-(1,5-dimethyl-hex-4-enyl)-thiazol-2-yl]-amide;
2-(2-Hydroxy-2-phenyl-acetylamino)-pentanoic acid [5-(1,5-dimethyl-hex-4-enyl)-thiazol-2-yl]-amide;
2-[2-(3,5-Difluoro-phenyl)-acetylamino]-pentanoic acid [5-(5-hydroxy-1,5-dimethyl-hexyl)-thiazol-2-yl]-amide;
2-[2-(3,5-Difluoro-phenyl)-acetylamino]-pentanoic acid [5-(5-methoxy-1,5-dimethyl-hexyl)-thiazol-2-yl]-amide;
2-[2-(3,5-Difluoro-phenyl)-acetylamino]-pentanoic acid [5-(1,5-dimethyl-hex-4-enyl)-thiazol-2-yl]-amide;
2-[2-(3,5-Difluoro-phenyl)-2-hydroxy-acetylamino]-pentanoic acid [5-(5-hydroxy-1,5-dimethyl-hexyl)-thiazol-2-yl]-amide;
2-[2-(3,5-Difluoro-phenyl)-acetylamino]-pentanoic acid {4-methyl-5-[1-(2,2,2-trifluoro-ethylamino)-ethyl]-thiazol-2-yl}-amide;
2-[2-(3,5-Difluoro-phenyl)-acetylamino]-pentanoic acid [5-(1-dimethylamino-ethyl)-4-methyl-thiazol-2-yl]-amide;
2-[2-(3,5-Difluoro-phenyl)-acetylamino]-pentanoic acid {5-[1-(2-hydroxy-ethylamino)-ethyl]-4-methyl-thiazol-2-yl}-amide;
2-[2-(3,5-Difluoro-phenyl)-acetylamino]-pentanoic acid [5-(1-isobutylamino-ethyl)-4-methyl-thiazol-2-yl]-amide;
2-[(2-{2-[2-(3,5-Difluoro-phenyl)-acetylamino]-pentanoylamino}-thiazol-5-ylmethyl)-amino]-pentanoic acid methyl ester;
2-[2-(3,5-Difluoro-phenyl)-acetylamino]-pentanoic acid [5-(1-isopropylamino-ethyl)-thiazol-2-yl]-amide;
2-[2-(3,5-Difluoro-phenyl)-acetylamino]-pentanoic acid [5-(1-benzylamino-ethyl)-thiazol-2-yl]-amide;
2-[2-(3,5-Difluoro-phenyl)-acetylamino]-pentanoic acid {5-[1-(3,3-dimethyl-butylamino)-ethyl]-thiazol-2-yl}-amide;
2-[2-(3,5-Difluoro-phenyl)-acetylamino]-pentanoic acid {5-[1-(3-methyl-butylamino)-ethyl]-thiazol-2-yl}-amide;
2-[2-(3,5-Difluoro-phenyl)-acetylamino]-pentanoic acid (5-ethyl-4-methyl-thiazol-2-yl)-amide;
2-[2-(3,5-Difluoro-phenyl)-acetylamino]-pentanoic acid [4-methyl-5-(1-methylamino-ethyl)-thiazol-2-yl]-amide;
2-[2-(3,5-Difluoro-phenyl)-acetylamino]-pentanoic acid [4-methyl-5-(1-methylamino-ethyl)-thiazol-2-yl]-amide;
2-[(2-{2-[2-(3,5-Difluoro-phenyl)-acetylamino]-pentanoylamino}-thiazol-5-ylmethyl)-amino]-pentanoic acid;
2-[2-(3,5-Difluoro-phenyl)-acetylamino]-pentanoic acid {5-[1-(2-hydroxy-ethylamino)-ethyl]-thiazol-2-yl}-amide;
2-[2-(3,5-Difluoro-phenyl)-acetylamino]-pentanoic acid [5-(1-phenethylamino-ethyl)-thiazol-2-yl]-amide;
2-[2-(3,5-Difluoro-phenyl)-acetylamino]-pentanoic acid [5-(1-morpholin-4-yl-ethyl)-thiazol-2-yl]-amide;
2-[2-(3,5-Difluoro-phenyl)-acetylamino]-pentanoic acid (5-trifluoroacetyl-thiazol-2-yl)-amide;
2-[2-(3,5-Difluoro-phenyl)-acetylamino]-pentanoic acid [5-(1-hydroxy-3,3-dimethoxy-1-methyl-propyl)-thiazol-2-yl]-amide;
2-[2-(3,5-Difluoro-phenyl)-acetylamino]-pentanoic acid [5-(2,2,2-trifluoro-1-hydroxy-ethyl)-thiazol-2-yl]-amide;
2-[2-(3,5-Difluoro-phenyl)-acetylamino]-pentanoic acid {5-[1-(1-benzyl-pyrrolidin-3-ylamino)-ethyl]-thiazol-2-yl}-amide;
2-[2-(3,5-Difluoro-phenyl)-acetylamino]-pentanoic acid {5-[1-(2-methoxy-ethylamino)-ethyl]-thiazol-2-yl}-amide;
2-[2-(3,5-Difluoro-phenyl)-acetylamino]-pentanoic acid [5-(1-isobutylamino-ethyl)-4-methyl-thiazol-2-yl]-amide;
2-[2-(3,5-Difluoro-phenyl)-acetylamino]-pentanoic acid [5-(1-isobutylamino-ethyl)-4-methyl-thiazol-2-yl]-amide;
2-[2-(3,5-Difluoro-phenyl)-acetylamino]-pentanoic acid [5-(1-propylamino-ethyl)-thiazol-2-yl]-amide;
2-[2-(3,5-Difluoro-phenyl)-acetylamino]-pentanoic acid {5-[1-(3,3-dimethyl-butylamino)-2,2,2-trifluoro-ethyl]-thiazol-2-yl}-amide;
2-Benzenesulfonylamino-pentanoic acid [5-(1-ethyl-propyl)-thiazol-2-yl]-amide; and
2-(4-Chloro-benzenesulfonylamino)-pentanoic acid [5-(5-hydroxy-1,5-dimethyl-hexyl)-thiazol-2-yl]-amide;
and pharmaceutically acceptable salts thereof.

23. A pharmaceutical composition for treating in a mammal a disease or condition associated with Aβ-peptide production, which pharmaceutical composition comprises a compound according to claim 1 a) in an amount effective in inhibiting Aβ-production, or b) in an amount effective in inhibiting said disease or condition, and a pharmaceutically acceptable carrier.

24. A method for treating in a mammal a disease or condition selected from Alzheimer's disease, hereditary cerebral hemorrhage with amyloidosis, cerebral amyloid angiopathy, a prion-mediated disease, inclusion body myositis, stroke, and Down's Syndrome, which method comprises administering to said mammal a) an amount of a compound according to claim 1 effective in inhibiting Aβ-production, or b) an amount of a compound according to any of claims 1-12 effective in treating said disease or condition.

25. A method for treating dementia, including Alzheimer's disease, in a mammal, which method comprises administering to the mammal an effective amount of a compound according to claim 1 and another drug, either separately or as part of a single pharmaceutical composition, wherein the other drug is selected from a memory enhancement agent, an antidepressant agent, an anxiolytic, an antipsychotic agent, a sleep disorder agent, an anti-inflammatory agent, an anti-oxidant agent, a cholesterol modulating agent, or an anti-hypertension agent.

26. A method of synthesizing a compound of Formula 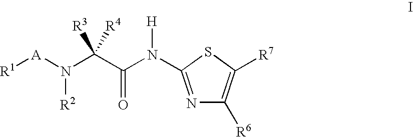
or a pharmaceutically acceptable salt thereof,
wherein:
A is selected from —C(═O)C(═O)—, —C(═O)NR⁹—, —C(═O)Z-, —C(═S)Z-, —C(═NR⁵)Z-, and —S(O)₂—;
wherein Z is —CH₂—, —CH(OH)—, —CH(OC(═O)R¹¹)—, —CH(NH₂)—, —CH(CH₂(OH))—, —CH(CH(C₁-C₄ alkyl)(OH))—, or —CH(C(C₁-C₄ alkyl)(C₁-C₄ alkyl)(OH))—, for example —CH(C(CH₃)(CH₃)(OH))— or —CH(C(CH₃)(CH₂CH₃)(OH))—;
R¹ is selected from C₁-C₂₀ alkyl and —C₁-C₂₀ alkoxy, C₃-C₈ cycloalkyl, (C₄-C₈)cycloalkenyl, (C₅-C₁₁)bi- or tricycloalkyl, (C₇-C₁₁)bi- or tricycloalkenyl, (3-8 membered) heterocycloalkyl, (C₆-C₁₄)aryl, or (5-14 membered) heteroaryl, wherein said alkyl and alkoxy each optionally contains from one to five double or triple bonds, and wherein each hydrogen atom of said alkyl and alkoxy is optionally replaced with a fluorine;
wherein when R¹ is alkyl or alkoxy, R¹ is optionally substituted with from one to three substituents R^{1a}, and wherein when R¹ is cycloalkyl, cycloalkenyl, bi- or tricycloalkyl, bi- or tricycloalkenyl, heterocycloalkyl, aryl, or heteroaryl, then R¹ is optionally substituted with from one to three substituents R^{1b};
R^{1a} is in each instance independently selected from —OH, —C₁-C₆ alkyl independently optionally containing from one to three double or triple bonds, —C₁-C₆ alkoxy independently optionally containing from one to three double or triple bonds, —Cl, —F, —Br, —I, —CN, —NO₂, —NR⁹R¹⁰, —C(═O)NR⁹R¹⁰, —S(O)ₙNR⁹R¹⁰, —C(═O)R¹¹, —S(O)ₙR¹¹, —C(═O)OR¹², —C₃-C₈ cycloalkyl, —C₄-C₈ cycloalkenyl, -(C₅-C₁₁)bi- or tricycloalkyl, -(C₇-C₁₁)bi- or tricycloalkenyl, -(3-8 membered) heterocycloalkyl, -(C₆-C₁₄)aryl, -(5-14 membered) heteroaryl, -(C₆-C₁₄) aryloxy, and -(5-14 membered) heteroaryloxy, wherein said alkyl, alkoxy, cycloalkyl, cycloalkenyl, bi- or tricycloalkyl, bi- or tricycloalkenyl, heterocycloalkyl, aryl, heteroaryl, aryloxy, and heteroaryloxy are each independently optionally substituted with from one to three substituents R^{1b};
R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, and —C₁-C₆ alkyl independently optionally containing from one to three double or triple bonds and independently substituted with from one to six atoms independently selected from F, Cl, Br, and I;
R² is selected from —H, —C₁-C₄ alkyl optionally containing one or two double or triple bonds, —C(═O)(C₁-C₄ alkyl), —C₆-C₁₀ aryl, —SO₂—(C₆-C₁₀ aryl), and —SO₂—CH₂—(C₆-C₁₀ aryl), and R² is optionally substituted with from one to three substituents R^{1b};
R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);
R⁴ is H, D, F, or C₁-C₄ alkyl;
or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;
R⁵ is selected from —H, —C₁-C₆ alkyl optionally substituted with from one to three R^{1a}, and —C₆-C₁₀ aryl optionally substituted with from one to three R^{1a};
or R⁵ and R¹ may together optionally form a five to fourteen membered heteroaryl ring or a five to eight membered heterocycloalkyl ring, wherein said heteroaryl ring optionally contains one or two further heteroatoms independently selected from N, O, and S, and said heterocycloalkyl ring optionally contains one or two further heteroatoms independently selected from N—R⁹, O, and S(O)_{zero-2}, and wherein said heterocycloalkyl ring optionally contains from one to three double bonds, and wherein said heteroaryl or heterocycloalkyl ring is optionally substituted from one to three substituents R^{1b};
R⁶ is selected from —H, —C₁-C₂₀ alkyl, —Cl, —F, —Br, —I, —CN, —CF₃, —C(═O)R¹¹, —C(═O)OR¹², —S(O)ₙNR⁹R¹⁰, —S(O)ₙR¹¹, —C(═NR⁹)R¹⁵, -(C₃-C₁₂) cycloalkyl, -(C₄-C₁₂) cycloalkenyl, and —C₆-C₁₀ aryl, wherein said alkyl, alkylene, cycloalkyl, cycloalkenyl, and aryl of R⁶ are each optionally substituted with from one to three substituents R^{1b};
R⁷ is selected from H, —Cl, —F, —Br, —I, —CN, —NO₂, —NR¹⁴R¹⁵, —CF₃, —C(═O)NR¹⁴R¹⁵, —C(═O)R¹³, —S(O)ₙR¹³, —C(═O)OR¹³, —C(═NR⁹)R¹⁵, —S(O)ₙNR¹⁴R¹⁵, —C₁-C₂₀ alkyl, —C₁-C₂₀ alkoxy, -(C_{zero}-C₄ alkylene)-(C₃-C₁₂ cycloalkyl), -(C_{zero}-C₄ alkylene)-((C₄-C₁₂)cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-((3-12 membered) heterocycloalkyl), -(C_{zero}-C₄ alkylene)-((7-20 membered) heterobi- or heterotricycloalkyl), -(C_{zero}-C₄ alkylene)-((C₆-C₁₄)aryl), and -(C_{zero}-C₄ alkylene)-((5-14 membered) heteroaryl); wherein R⁷ is optionally substituted with from one to three substituents independently selected from R^{1a}, -(CH₂)₁₋₁₀NR⁹R¹⁰, —C₃-C₁₂ cycloalkyl, -((4-12 membered) heterocycloalkyl), -(C₆-C₁₄) aryl, -((5-15 membered) heteroaryl), -(4-12 membered) heterocycloalkoxy), -(C₆-C₁₂) aryloxy and -((5-12 membered) heteroaryloxy); said cycloalkyl, cycloalkenyl, bi- or tricycloalkyl, bi- or tricycloalkenyl, heterocycloalkyl, aryl, and heteroaryl of R⁷ are each optionally and independently substituted with from one to six F; said alkyl, alkoxy, and alkylene of R⁷ each optionally contains from one to five double or triple bonds; and each hydrogen atom of said alkyl, alkoxy, and alkylene of R⁷ is independently optionally replaced with a fluorine;
or R⁶ and R⁷ may together optionally form a -(C₆-C₁₀) aryl ring, -(C₆-C₈) cycloalkyl or cycloalkenyl ring, a five to eight membered heterocycloalkyl or heterocycloalkenyl ring, a -(C₁₀-C₁₄) membered bicycloalkyl or bicycloalkenyl ring, or a ten to fourteen membered heterobicycloalkyl or heterobicycloalkenyl ring fused to the thiazole ring of Formula I, wherein from one to three members of said heterocycloalkyl and heterocycloalkenyl rings, and from one to five members of said heterobicycloalkyl and heterobicycloalkenyl rings are selected independently from N—R⁹, O and S(O)_{zero-2}, and wherein said aryl, cycloalkyl, cycloalkenyl, heterocycloalkyl, heterocycloalkenyl, bicycloalkyl, bicycloalkenyl, heterobicycloalkyl, and heterobicycloalkenyl rings optionally are substituted with from one to three R^{1b};
R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇-C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
or NR⁹R¹⁰ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};
R¹³ is selected from H, —C₁-C₆ alkyl optionally containing from one to three double or triple bonds and wherein each hydrogen is independently optionally replaced with a fluorine, -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), and -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-((3-12 membered) heterocycloalkyl), -(C_{zero}-C₄ alkylene)-((7-20 membered) heterobi- or heterotricycloalkyl), and -(C_{zero}-C₄ alkylene)-((5-14 membered) heteroaryl), and R¹³ is optionally substituted with from one to three substituents R^{1b};
R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;
or NR¹⁴R¹⁵ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I; and
n is in each instance an integer independently selected from zero, 1, 2, and 3;
which method comprises reacting a compound of Formula 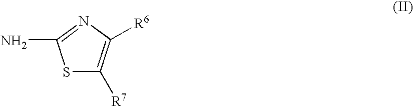
wherein R⁶ and R⁷ are as defined above,
with a compound of Formula 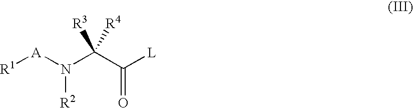
wherein R¹, R², R³, R⁴, and A are as defined above, and L is hydroxy or a suitable leaving group.

27. A method of synthesizing a compound of Formula 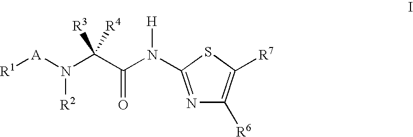
or a pharmaceutically acceptable salt thereof,
wherein:
A is selected from —C(═O)C(═O)—, —C(═O)NR⁹—, —C(═O)Z-, —C(═S)Z-, —C(═NR⁵)Z-, and —S(O)₂—;
wherein Z is —CH₂—, —CH(OH)—, —CH(OC(═O)R¹¹)—, —CH(NH₂)—, —CH(CH₂(OH))—, —CH(CH(C₁-C₄ alkyl)(OH))—, or —CH(C(C₁-C₄ alkyl)(C₁-C₄ alkyl)(OH))—, for example —CH(C(CH₃)(CH₃)(OH))— or —CH(C(CH₃)(CH₂CH₃)(OH))—;
R¹ is selected from C₁-C₂₀ alkyl and —C₁-C₂₀ alkoxy, C₃-C₈ cycloalkyl, (C₄-C₈)cycloalkenyl, (C₅-C₁₁)bi- or tricycloalkyl, (C₇-C₁₁)bi- or tricycloalkenyl, (3-8 membered) heterocycloalkyl, (C₆-C₁₄)aryl, or (5-14 membered) heteroaryl, wherein said alkyl and alkoxy each optionally contains from one to five double or triple bonds, and wherein each hydrogen atom of said alkyl and alkoxy is optionally replaced with a fluorine;
wherein when R¹ is alkyl or alkoxy, R¹ is optionally substituted with from one to three substituents R^{1a}, and wherein when R¹ is cycloalkyl, cycloalkenyl, bi- or tricycloalkyl, bi- or tricycloalkenyl, heterocycloalkyl, aryl, or heteroaryl, then R¹ is optionally substituted with from one to three substituents R^{1b};
R^{1a} is in each instance independently selected from —OH, —C₁-C₆ alkyl independently optionally containing from one to three double or triple bonds, —C₁-C₆ alkoxy independently optionally containing from one to three double or triple bonds, —Cl, —F, —Br, —I, —CN, —NO₂, —NR⁹R¹⁰, —C(═O)NR⁹R¹⁰, —S(O)ₙNR⁹R¹⁰, —C(═O)R¹¹, —S(O)ₙR¹¹, —C(═O)OR¹², —C₃-C₈ cycloalkyl, —C₄-C₈ cycloalkenyl, -(C₅-C₁₁)bi- or tricycloalkyl, -(C₇-C₁₁)bi- or tricycloalkenyl, -(3-8 membered) heterocycloalkyl, -(C₆-C₁₄)aryl, -(5-14 membered) heteroaryl, -(C₆-C₁₄) aryloxy, and -(5-14 membered) heteroaryloxy, wherein said alkyl, alkoxy, cycloalkyl, cycloalkenyl, bi- or tricycloalkyl, bi- or tricycloalkenyl, heterocycloalkyl, aryl, heteroaryl, aryloxy, and heteroaryloxy are each independently optionally substituted with from one to three substituents R^{1b};
R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, and —C₁-C₆ alkyl independently optionally containing from one to three double or triple bonds and independently substituted with from one to six atoms independently selected from F, Cl, Br, and I;
R² is selected from —H, —C₁-C₄ alkyl optionally containing one or two double or triple bonds, —C(═O)(C₁-C₄ alkyl), —C₆-C₁₀ aryl, —SO₂—(C₆-C₁₀ aryl), and —SO₂—CH₂—(C₆-C₁₀ aryl), and R² is optionally substituted with from one to three substituents R^{1b};
R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);
R⁴ is H, D, F, or C₁-C₄ alkyl;
or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;
R⁵ is selected from —H, —C₁-C₆ alkyl optionally substituted with from one to three R^{1a}, and —C₆-C₁₀ aryl optionally substituted with from one to three R^{1a};
or R⁵ and R¹ may together optionally form a five to fourteen membered heteroaryl ring or a five to eight membered heterocycloalkyl ring, wherein said heteroaryl ring optionally contains one or two further heteroatoms independently selected from N, O, and S, and said heterocycloalkyl ring optionally contains one or two further heteroatoms independently selected from N—R⁹, O, and S(O)_{zero-2}, and wherein said heterocycloalkyl ring optionally contains from one to three double bonds, and wherein said heteroaryl or heterocycloalkyl ring is optionally substituted from one to three substituents R^{1b};
R⁶ is selected from —H, —C₁-C₂₀ alkyl, —Cl, —F, —Br, —I, —CN, —CF₃, —C(═O)R¹¹, —C(═O)OR¹², —S(O)ₙNR⁹R¹⁰, —S(O)ₙR¹¹, —C(═NR⁹)R¹⁵, -(C₃-C₁₂) cycloalkyl, -(C₄-C₁₂) cycloalkenyl, and —C₆-C₁₀ aryl, wherein said alkyl, alkylene, cycloalkyl, cycloalkenyl, and aryl of R⁶ are each optionally substituted with from one to three substituents R^{1b};
R⁷ is selected from H, —Cl, —F, —Br, —I, —CN, —NO₂, —NR¹⁴R¹⁵, —CF₃, —C(═O)NR¹⁴R¹⁵, —C(═O)R¹³, —S(O)ₙR¹³, —C(═O)OR¹³, —C(═NR⁹)R¹⁵, —S(O)ₙNR¹⁴R¹⁵, —C₁-C₂₀ alkyl, —C₁-C₂₀ alkoxy, -(C_{zero}-C₄ alkylene)-(C₃-C₁₂ cycloalkyl), -(C_{zero}-C₄ alkylene)-((C₄-C₁₂)cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-((3-12 membered) heterocycloalkyl), -(C_{zero}-C₄ alkylene)-((7-20 membered) heterobi- or heterotricycloalkyl), -(C_{zero}-C₄ alkylene)-((C₆-C₁₄)aryl), and -(C_{zero}-C₄ alkylene)-((5-14 membered) heteroaryl); wherein R⁷ is optionally substituted with from one to three substituents independently selected from R^{1a}, -(CH₂)₁₋₁₀NR⁹R¹⁰, —C₃-C₁₂ cycloalkyl, -((4-12 membered) heterocycloalkyl), -(C₆-C₁₄) aryl, -((5-15 membered) heteroaryl), -(4-12 membered) heterocycloalkoxy), -(C₆-C₁₂) aryloxy and -((5-12 membered) heteroaryloxy); said cycloalkyl, cycloalkenyl, bi- or tricycloalkyl, bi- or tricycloalkenyl, heterocycloalkyl, aryl, and heteroaryl of R⁷ are each optionally and independently substituted with from one to six F; said alkyl, alkoxy, and alkylene of R⁷ each optionally contains from one to five double or triple bonds; and each hydrogen atom of said alkyl, alkoxy, and alkylene of R⁷ is independently optionally replaced with a fluorine;
or R⁶ and R⁷ may together optionally form a -(C₆-C₁₀) aryl ring, -(C₆-C₈) cycloalkyl or cycloalkenyl ring, a five to eight membered heterocycloalkyl or heterocycloalkenyl ring, a -(C₁₀-C₁₄) membered bicycloalkyl or bicycloalkenyl ring, or a ten to fourteen membered heterobicycloalkyl or heterobicycloalkenyl ring fused to the thiazole ring of Formula I, wherein from one to three members of said heterocycloalkyl and heterocycloalkenyl rings, and from one to five members of said heterobicycloalkyl and heterobicycloalkenyl rings are selected independently from N—R⁹, O and S(O)_{zero-2}, and wherein said aryl, cycloalkyl, cycloalkenyl, heterocycloalkyl, heterocycloalkenyl, bicycloalkyl, bicycloalkenyl, heterobicycloalkyl, and heterobicycloalkenyl rings optionally are substituted with from one to three R^{1b};
R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇-C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
or NR⁹R¹⁰ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};
R¹³ is selected from H, —C₁-C₆ alkyl optionally containing from one to three double or triple bonds and wherein each hydrogen is independently optionally replaced with a fluorine, -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), and -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-((3-12 membered) heterocycloalkyl), -(C_{zero}-C₄ alkylene)-((7-20 membered) heterobi- or heterotricycloalkyl), and -(C_{zero}-C₄ alkylene)-((5-14 membered) heteroaryl), and R¹³ is optionally substituted with from one to three substituents R^{1b};
R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;
or NR¹⁴R¹⁵ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I; and
n is in each instance an integer independently selected from zero, 1, 2, and 3;
which method comprises reacting a compound of Formula IV 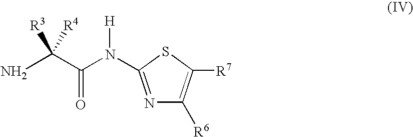
wherein R³, R⁴, R⁶ and R⁷ are as defined above;
with a compound of Formula
R¹-A-L (V)
wherein R¹ and A are as defined above, and L is hydroxy or a suitable leaving group;
or wherein R¹ is as defined above, and A-L is an alkyl ester or an aryl ester.

28. A method according to claim 27, wherein the compound of Formula IV is obtained by reacting a compound of Formula 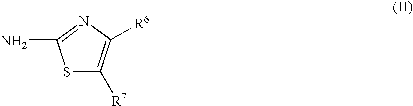
wherein R⁶ and R⁷ are as recited in claim 2;
with a compound of Formula 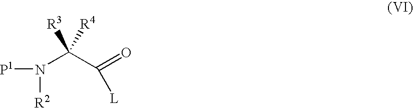
wherein R², R³, and R⁴ are as recited in claim 2; L is hydroxy or a suitable leaving group; and P¹ is an amino protecting group.

29. A compound of Formula 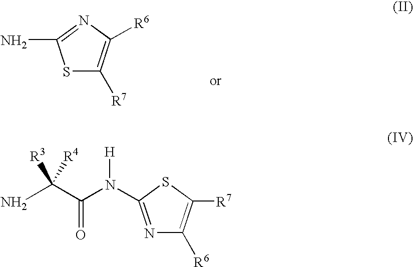
wherein
R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);
R⁴ is H, D, F, or C₁-C₄ alkyl;
or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;
R⁶ is selected from —H, —C₁-C₂₀ alkyl, —Cl, —F, —Br, —I, —CN, —CF₃, —C(═O)R¹¹, —C(═O)OR¹², —S(O)ₙNR⁹R¹⁰, —S(O)ₙR¹¹, —C(═NR⁹)R¹⁵, -(C₃-C₁₂) cycloalkyl, -(C₄-C₁₂) cycloalkenyl, and —C₆-C₁₀ aryl, wherein said alkyl, alkylene, cycloalkyl, cycloalkenyl, and aryl of R⁶ are each optionally substituted with from one to three substituents R^{1b};
R⁷ is selected from H, —Cl, —F, —Br, —I, —CN, —NO₂, —NR¹⁴R¹⁵, —CF₃, —C(═O)NR¹⁴R¹⁵, —C(═O)R¹³, —S(O)ₙR¹³, —C(═O)OR¹³, —C(═NR⁹)R¹⁵, —S(O)ₙNR¹⁴R¹⁵, —C₁-C₂₀ alkyl, —C₁-C₂₀ alkoxy, -(C_{zero}-C₄ alkylene)-(C₃-C₁₂ cycloalkyl), -(C_{zero}-C₄ alkylene)-((C₄-C₁₂)cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-((3-12 membered) heterocycloalkyl), -(C_{zero}-C₄ alkylene)-((7-20 membered) heterobi- or heterotricycloalkyl), -(C_{zero}-C₄ alkylene)-((C₆-C₁₄)aryl), and -(C_{zero}-C₄ alkylene)-((5-14 membered) heteroaryl); wherein R⁷ is optionally substituted with from one to three substituents independently selected from R^{1a}, -(CH₂)₁₋₁₀NR⁹R¹⁰, —C₃-C₁₂ cycloalkyl, -((4-12 membered) heterocycloalkyl), -(C₆-C₁₄) aryl, -((5-15 membered) heteroaryl), -(4-12 membered) heterocycloalkoxy), -(C₆-C₁₂) aryloxy and -((5-12 membered) heteroaryloxy); said cycloalkyl, cycloalkenyl, bi- or tricycloalkyl, bi- or tricycloalkenyl, heterocycloalkyl, aryl, and heteroaryl of R⁷ are each optionally and independently substituted with from one to six F; said alkyl, alkoxy, and alkylene of R⁷ each optionally contains from one to five double or triple bonds; and each hydrogen atom of said alkyl, alkoxy, and alkylene of R⁷ is independently optionally replaced with a fluorine;
or R⁶ and R⁷ may together optionally form a -(C₆-C₁₀) aryl ring, -(C₆-C₈) cycloalkyl or cycloalkenyl ring, a five to eight membered heterocycloalkyl or heterocycloalkenyl ring, a -(C₁₀-C₁₄) membered bicycloalkyl or bicycloalkenyl ring, or a ten to fourteen membered heterobicycloalkyl or heterobicycloalkenyl ring fused to the thiazole ring of Formula I, wherein from one to three members of said heterocycloalkyl and heterocycloalkenyl rings, and from one to five members of said heterobicycloalkyl and heterobicycloalkenyl rings are selected independently from N—R⁹, O and S(O)_{zero-2}, and wherein said aryl, cycloalkyl, cycloalkenyl, heterocycloalkyl, heterocycloalkenyl, bicycloalkyl, bicycloalkenyl, heterobicycloalkyl, and heterobicycloalkenyl rings optionally are substituted with from one to three R^{1b};
R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇-C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
or NR⁹R¹⁰ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};
R¹³ is selected from H, —C₁-C₆ alkyl optionally containing from one to three double or triple bonds and wherein each hydrogen is independently optionally replaced with a fluorine, -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), and -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-((3-12 membered) heterocycloalkyl), -(C_{zero}-C₄ alkylene)-((7-20 membered) heterobi- or heterotricycloalkyl), and -(C_{zero}-C₄ alkylene)-((5-14 membered) heteroaryl), and R¹³ is optionally substituted with from one to three substituents R^{1b};
R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;
or NR¹⁴R¹⁵ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I;
R^{1a} is in each instance independently selected from —OH, —C₁-C₆ alkyl independently optionally containing from one to three double or triple bonds, —C₁-C₆ alkoxy independently optionally containing from one to three double or triple bonds, —Cl, —F, —Br, —I, —CN, —NO₂, —NR⁹R¹⁰, —C(═O)NR⁹R¹⁰, —S(O)ₙNR⁹R¹⁰, —C(═O)R¹¹, —S(O)ₙR¹¹, —C(═O)OR¹², —C₃-C₈ cycloalkyl, —C₄-C₈ cycloalkenyl, -(C₅-C₁₁)bi- or tricycloalkyl, -(C₇-C₁₁)bi- or tricycloalkenyl, -(3-8 membered) heterocycloalkyl, -(C₆-C₁₄)aryl, -(5-14 membered) heteroaryl, -(C₆-C₁₄) aryloxy, and -(5-14 membered) heteroaryloxy, wherein said alkyl, alkoxy, cycloalkyl, cycloalkenyl, bi- or tricycloalkyl, bi- or tricycloalkenyl, heterocycloalkyl, aryl, heteroaryl, aryloxy, and heteroaryloxy are each independently optionally substituted with from one to three substituents R^{1b};
R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, and —C₁-C₆ alkyl independently optionally containing from one to three double or triple bonds and independently substituted with from one to six atoms independently selected from F, Cl, Br, and I; and
n is in each instance an integer independently selected from zero, 1, 2, and 3.

30. A compound of Formula 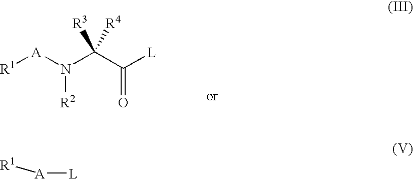
wherein:
A is selected from —C(═O)C(═O)—, —C(═O)NR⁹—, —C(═O)Z-, —C(═S)Z-, —C(═NR⁵)Z-, and —S(O)₂—;
wherein Z is —CH₂—, —CH(OH)—, —CH(OC(═O)R¹¹)—, —CH(NH₂)—, —CH(CH₂(OH))—, —CH(CH(C₁-C₄ alkyl)(OH))—, or —CH(C(C₁-C₄ alkyl)(C₁-C₄ alkyl)(OH))—, for example —CH(C(CH₃)(CH₃)(OH))— or —CH(C(CH₃)(CH₂CH₃)(OH))—;
R¹ is selected from C₁-C₂₀ alkyl and —C₁-C₂₀ alkoxy, C₃-C₈ cycloalkyl, (C₄-C₈)cycloalkenyl, (C₅-C₁₁)bi- or tricycloalkyl, (C₇-C₁₁)bi- or tricycloalkenyl, (3-8 membered) heterocycloalkyl, (C₆-C₁₄)aryl, or (5-14 membered) heteroaryl, wherein said alkyl and alkoxy each optionally contains from one to five double or triple bonds, and wherein each hydrogen atom of said alkyl and alkoxy is optionally replaced with a fluorine;
wherein when R¹ is alkyl or alkoxy, R¹ is optionally substituted with from one to three substituents R^{1a}, and wherein when R¹ is cycloalkyl, cycloalkenyl, bi- or tricycloalkyl, bi- or tricycloalkenyl, heterocycloalkyl, aryl, or heteroaryl, then R¹ is optionally substituted with from one to three substituents R^{1b};
R^{1a} is in each instance independently selected from —OH, —C₁-C₆ alkyl independently optionally containing from one to three double or triple bonds, —C₁-C₆ alkoxy independently optionally containing from one to three double or triple bonds, —Cl, —F, —Br, —I, —CN, —NO₂, —NR⁹R¹⁰, —C(═O)NR⁹R¹⁰, —S(O)ₙNR⁹R¹⁰, —C(═O)R¹¹, —S(O)ₙR¹¹, —C(═O)OR¹², —C₃-C₈ cycloalkyl, —C₄-C₈ cycloalkenyl, -(C₅-C₁₁)bi- or tricycloalkyl, -(C₇-C₁₁)bi- or tricycloalkenyl, -(3-8 membered) heterocycloalkyl, -(C₆-C₁₄)aryl, -(5-14 membered) heteroaryl, -(C₆-C₁₄) aryloxy, and -(5-14 membered) heteroaryloxy, wherein said alkyl, alkoxy, cycloalkyl, cycloalkenyl, bi- or tricycloalkyl, bi- or tricycloalkenyl, heterocycloalkyl, aryl, heteroaryl, aryloxy, and heteroaryloxy are each independently optionally substituted with from one to three substituents R^{1b};
R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, and —C₁-C₆ alkyl independently optionally containing from one to three double or triple bonds and independently substituted with from one to six atoms independently selected from F, Cl, Br, and I;
R² is selected from —H, —C₁-C₄ alkyl optionally containing one or two double or triple bonds, —C(═O)(C₁-C₄ alkyl), —C₆-C₁₀ aryl, —SO₂—(C₆-C₁₀ aryl), and —SO₂—CH₂—(C₆-C₁₀ aryl), and R² is optionally substituted with from one to three substituents R^{1b};
R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);
R⁴ is H, D, F, or C₁-C₄ alkyl;
or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;
R⁵ is selected from —H, —C₁-C₆ alkyl optionally substituted with from one to three R^{1a}, and —C₆-C₁₀ aryl optionally substituted with from one to three R^{1a};
or R⁵ and R¹ may together optionally form a five to fourteen membered heteroaryl ring or a five to eight membered heterocycloalkyl ring, wherein said heteroaryl ring optionally contains one or two further heteroatoms independently selected from N, O, and S, and said heterocycloalkyl ring optionally contains one or two further heteroatoms independently selected from N—R⁹, O, and S(O)_{zero-2}, and wherein said heterocycloalkyl ring optionally contains from one to three double bonds, and wherein said heteroaryl or heterocycloalkyl ring is optionally substituted from one to three substituents R^{1b};
R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇-C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
or NR⁹R¹⁰ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};
R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;
or NR¹⁴R¹⁵ can independently optionally form a heterocycloalkyl moiety of from four to seven ring members, said heterocycloalkyl moiety independently optionally comprising one or two further heteroatoms independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I; and
n is in each instance an integer independently selected from zero, 1, 2, and 3; and
L is hydroxy or a suitable leaving group; or
A-L is an alkyl ester or an aryl ester.

31. A compound of Formula III according to claim 30, wherein L is hydroxy or a halogen atom.

32. A compound of Formula III according to claim 30, wherein A-L is an alkyl ester or an aryl ester.

33. A compound of Formula V according to claim 30, wherein L is hydroxy or a halogen atom.

34. A compound of Formula V according to claim 30, wherein A-L is an alkyl ester or an aryl ester.

35. A compound of Formula 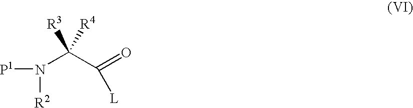
wherein:
R² is selected from —H, —C₁-C₄ alkyl optionally containing one or two double or triple bonds, —C(═O)(C₁-C₄ alkyl), —C₆-C₁₀ aryl, —SO₂—(C₆-C₁₀ aryl), and —SO₂—CH₂—(C₆-C₁₀ aryl), and R² is optionally substituted with from one to three substituents R^{1b};
R³ is selected from C₁-C₆ alkyl, —C₂-C₆ alkenyl, —C₂-C₆ alkynyl, -(C_{zero}-C₄ alkylene)-(C₃-C₆ cycloalkyl), and -(C_{zero}-C₄ alkylene)-(C₃-C₆ cycloalkenyl), wherein said alkyl, alkenyl and alkynyl are each optionally substituted with a substituent selected from —OH, C₁-C₄ alkoxy, and —S—(C₁-C₄ alkyl);
R⁴ is H, D, F, or C₁-C₄ alkyl;
or R³ and R⁴ may together optionally form a cyclopropyl, cyclobutyl, cyclopentyl, cyclohexyl, morpholino, piperidino, or perhydro-2H-pyran moiety, wherein said moiety formed by R³ and R⁴ is optionally substituted with one to three substituents independently selected from —OH, —Cl, —F, —CN, —CF₃, methyl, ethyl, methoxy, ethoxy, allyl, and —OCF₃;
R^{1b} is in each instance independently selected from —Cl, —F, —Br, —I, —CN, —NO₂, —NR⁹R¹⁰, —C(═)ONR⁹R¹⁰, —C(═O)R¹¹, —C(═O)OR¹², —S(O)ₙR¹¹, —S(O)ₙNR⁹R¹⁰, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C₆-C₁₄) aryl, and —C₁-C₆ alkyl independently optionally containing from one to three double or triple bonds and independently substituted with from one to six atoms independently selected from F, Cl, Br, and I;
R⁹ and R¹⁰ are each independently selected from —H, —OH, —C₁-C₆ alkyl independently optionally containing from one to three double or triple bonds and wherein each hydrogen is independently optionally replaced with a fluorine, —C₁-C₆ alkoxy independently optionally containing from one to three double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), -(C_{zero}-C₄ alkylene)-((C₇C₁₁)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each optionally independently substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
or NR⁹R¹⁰ can independently optionally form a heterocycloalkyl moiety of from four to seven ring members, said heterocycloalkyl moiety independently optionally comprising one or two furthers independently selected from N—R⁹, O, and S(O)_{zero-2}, and independently optionally containing from one to three double bonds, and said heterocycloalkyl moiety independently optionally substituted with from one to three substituents independently selected from —Cl, —F, —Br, —I, —CN, —NO₂, —NR¹⁴R¹⁵, —C(═)ONR¹⁴R¹⁵, —C(═O)R¹¹, —C(═O)OR¹², —S(O)ₙR¹¹, —S(O)ₙNR¹⁴R¹⁵, —OH, —C₁-C₆ alkyl independently optionally containing from one to three double or triple bonds, —C₁-C₆ alkoxy independently optionally containing from one to three double or triple bonds, —C₁-C₆ hydroxyalkyl independently optionally containing from one to three double or triple bonds, -(C₆-C₁₄) aryloxy, -(5-14 membered) heteroaryloxy, -(C_{zero}-C₄)-((C₆-C₁₄) aryl), -(C_{zero}-C₄)-(5-14 membered heteroaryl), and —C₁-C₆ alkyl independently optionally containing from one to three double or triple bonds and independently substituted with from one to six atoms independently selected from F, Cl, Br, and I;
R¹¹ and R¹² are each independently selected from H, —C₁-C₆ alkyl, -(C_{zero}-C₄ alkylene)-(C₃-C₈ cycloalkyl), -(C_{zero}-C₄ alkylene)-(C₄-C₈ cycloalkenyl), -(C_{zero}-C₄ alkylene)-((C₅-C₁₁)bi- or tricycloalkyl), and -(C_{zero}-C₄ alkylene)-((C₇-C₁₁)bi- or tricycloalkenyl), -(C_{zero}-C₄ alkylene)-(C₆-C₁₀ aryl), -(C_{zero}-C₄ alkylene)-((3-8 membered) heterocycloalkyl), and -(C_{zero}-C₄ alkylene)-((5-14 membered) heteroaryl), and R¹¹ and R¹² are independently optionally substituted with from one to three R^{1b};
R¹⁴ and R¹⁵ are each independently selected from —H, —C₁-C₂₀ alkyl independently optionally containing from one to five double or triple bonds and wherein each hydrogen is independently optionally replaced with a fluorine, —C(═O)R¹¹, —S(O)ₙR¹¹, —C(═O)OR¹², —S(O)ₙNR¹¹R¹², -(C_{zero}-C₄ alkylene)-(C₃-C₁₂ cycloalkyl), -(C_{zero}-C₄ alkylene)-(C₄-C₁₂ cycloalkenyl), -(C_{zero}-C₄ alkylene)-((C₅-C₂₀)bi- or tricycloalkyl), -(C_{zero}-C₄ alkylene)-((C₇-C₂₀)bi- or tricycloalkenyl), -(C_{zero}-C₄ alkylene)-(C₆-C₁₄ aryl), -(C_{zero}-C₄ alkylene)-(3-8 membered heterocycloalkyl), and -(C_{zero}-C₄ alkylene)-(5-14 membered heteroaryl), wherein said cycloalkyl, cycloalkenyl, bi- or tricycloalkyl, bi- or tricycloalkenyl, aryl, heterocycloalkyl, and heteroaryl are each independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently substituted with from one to six atoms independently selected from F, Cl, Br, and I and independently optionally containing from one to three double or triple bonds;
or NR¹⁴R¹⁵ can independently optionally form a heterocycloalkyl moiety of from four to seven ring members, said heterocycloalkyl moiety independently optionally comprising one or two furthers independently selected from N—R⁹, O, and S(O)_{zero-2}, and independently optionally containing from one to three double bonds, and said heterocycloalkyl moiety independently optionally substituted with from one to three substituents independently selected from —C₁-C₆ alkyl independently optionally containing from one to three double or triple bonds and wherein each hydrogen is independently optionally replaced with fluorine, —Cl, —F, —Br, —I, —CN, —NO₂, —NH₂, —OH, —C(═O)H, —S(O)ₙH, —C(═O)OH, —C(═O)NH₂, —S(O)ₙNH₂, —C₁-C₆ alkoxy independently optionally containing from one to three double or triple bonds and wherein each hydrogen is independently optionally replaced with fluorine, —C₁-C₆ hydroxyalkyl independently optionally containing from one to three double or triple bonds and wherein each hydrogen is independently optionally replaced with a fluorine, -(5-14 membered) heteroaryloxy, -(C₆-C₁₄ aryloxy), -(C_{zero}-C₄ alkylene)-(C₆-C₁₄ aryl), -(C_{zero}-C₄ alkylene)-((5-14 membered) heteroaryl), and —C₁-C₆ alkyl independently optionally containing from one to three double or triple bonds and independently substituted with from one to six atoms independently selected from F, Cl, Br, and I;
n is in each instance an integer independently selected from zero, 1, 2, and 3;
L is hydroxy or a suitable leaving group; and
P¹ is an amino protecting group.
